(19) United States
(12) Patent Application Publication
(10) Pub. No.: US 2026/0226069 A1
(43) Pub. Date: Aug. 6, 2026

(54) GCN2 AND PERK KINASE MODULATORS AND METHODS OF USE THEREOF

(71) Applicant: Deciphera Pharmaceuticals, LLC, Waltham, MA (US)

(72) Inventors: Jeffery Zwicker, Waltham, MA (US); Salim Javed, Waltham, MA (US); Patrick Kearney, Waltham, MA (US); Yu Mi Ahn, Waltham, MA (US); Bertrand Le Bourdonnec, Waltham, MA (US); Daniel L. Flynn, Waltham, MA (US)

(21) Appl. No.: 19/393,975

(22) Filed: Nov. 19, 2025

Related U.S. Application Data
(63) Continuation of application No. PCT/US2024/031438, filed on May 29, 2024.
(60) Provisional application No. 63/504,801, filed on May 30, 2023.

(51) Int. Cl. C07D 487/04; A61K 31/4985; A61K 31/519; A61K 31/53; A61K 45/06; A61P 35/00; A61P 35/02

(52) U.S. Cl. CPC C07D 487/04; A61K 31/4985; A61K 31/519; A61K 31/53; A61K 45/06; A61P 35/00; A61P 35/02

(57) ABSTRACT

Described herein are compounds that are modulators of GCN2 kinase or PERK kinase, and methods of treating diseases, including diseases associated with GCN2 kinase or PERK kinase, with said compounds.

### CROSS REFERENCE

This application is a Continuation of International Application Number PCT/US2024/031438 filed May 29, 2024, which claims priority to U.S. Provisional Application No. 63/504,801 filed May 30, 2023, the contents of which are incorporated herein by reference in their entireties.

### REFERENCE TO A SEQUENCE LISTING XML

This application contains a Sequence Listing which has been submitted electronically in XML format. The Sequence Listing XML is incorporated herein by reference. Said XML file, created on Nov. 13, 2025, is named DCP-108USWOC1_SL.xml and is 7,442 bytes in size.

### BACKGROUND

Cancer cells need a continuous supply of nutrients to maintain their abnormal growth and rapid division. As part of these nutrients, amino acids are essential to support the high metabolic demands of tumor cells.

General control nonderepressible 2 (GCN2) is a serine/threonine protein kinase, one of the eukaryotic initiation factor 2a (eIF2α) kinases that are master regulators in the integrated stress response (ISR). The ISR is essential for maintaining cellular homeostasis under a wide range of stressors and is activated when cells adapt to stress conditions such as hypoxia and amino acid deprivation. The ISR is regulated by phosphorylation and activation of eIF2α kinases, including GCN2, that act as early responders to disturbances in cellular homeostasis. In addition to GCN2, there are three other eIF2α kinases family including PKR-like ER kinase (PERK), double-stranded RNA-dependent protein kinase (PKR), and heme-regulated eIF2α kinase (HRI). All four eIF2α kinases share extensive homology in their kinase catalytic domains but possess distinct regulatory domains. Each of the IF2α kinases responds to distinct environmental and physiological stresses, which reflect their unique regulatory mechanisms. PERK kinase is activated under stress conditions including ATP depletion and the unfolded protein response, and like GCN2, PERK kinase activation leads to up-regulation of the key ISR transcription factor ATF4.

Under conditions of essential amino-acid limitation or other stressors (UV irradiation, redox stress or proteasome inhibition), GCN2 phosphorylates eIF2α, which inhibits the formation of a new ternary complex and hence inhibition of mRNA translation initiation. While decreasing global mRNA translation, in tumor cells eIF2α phosphorylation also increases the translation of the ISR transcription factor ATF4, which increases the expression of many stress response genes including genes dedicated to providing amino acids to the tumor cell: i.e., amino acid synthesis enzymes and transporters that mediate influx of amino acids into the tumor cell. ATF4 is over-expressed in human solid and liquid tumors suggesting an important function in tumor progression.

Asparagine is an important amino acid involved in several biosynthetic pathways that significantly influence carcinogenesis and tumor biology. All cells need asparagine for their protein synthesis and growth. Normal cells will obtain most of their asparagine requirements through internal synthesis. Compared to normal cells, cancer cells require elevated amounts of asparagine to grow and proliferate, cannot produce that required amount themselves, and must rely on circulating asparagine in order to survive. Asparagine Synthetase (ASNS) catalyzes the synthesis of asparagine from aspartate and glutamine. L-asparaginase (ASNase) removes circulating asparagine, thereby depriving cancer cells of a key nutrient and causing them to die. The use of L-asparaginase, the first example of anti-cancer treatment targeting a tumor-specific metabolic feature, is a well-established treatment in pediatric acute lymphoblastic leukemia (ALL), but toxicity has limited its use beyond this patient population. The particularly low-level expression of ASNS in numerous ALL cell lines, as compared to that of normal cells, makes asparagine depletion an effective method of treatment due to the cells' unusual dependency on circulating serum asparagine as a necessary nutrition for growth. A poor response to asparaginase is associated with increased relapse risk. Other hematological and solid cancers express low levels of ASNS and, therefore, should also be asparagine auxotroph and asparaginase sensitive. Conversely, in some cancer types ASNS is overexpressed, promoting cell proliferation, chemoresistance, and a metastatic behavior. In case of asparaginase resistant cancers, the effect of blood asparagine depletion through L-asparaginase instead leads to significant ASNS overexpression to compensate, effectively nullifying the effect of the chemotherapy drug. Numerous studies have shown that ASNS is at the center of the cell response to amino acid deprivation and other forms of cellular stress. Through transcriptional regulation, the ASNS gene is a target of two signaling pathways aimed at ensuring cell survival. The first, named Amino Acid Response (AAR), is activated by the GCN2 kinase under conditions of imbalanced amino acid availability. The second pathway, named the Unfolded Protein Response (UPR), is activated by the PERK kinase under conditions of increased endoplasmic reticulum stress. The AAR and UPR pathways converge on the phosphorylation of eIF2α, which provokes the attenuation of global protein synthesis and, at the same time, the preferential translation of a selected population of mRNAs, including the transcription factor ATF4. ATF4 is the major factor for ASNS induction, working as a trans-activator through the binding to an enhancer element within ASNS promoter.

GCN2 sensitizes cancer cells with low basal level expression of ASNS to the antileukemic agent L-asparaginase in vitro and in vivo. Treatment with GCN2 inhibitors rendered acute lymphoblastic leukemia cells sensitive to L-asparaginase by preventing the induction of ASNS. GCN2 inhibitors exhibit synergistic antiproliferative effects with L-asparaginase in ASNS-low/deficient cancers. Therefore, combined treatment with GCN2 inhibitors and L-asparaginase shows promise for achieving improved outcomes in acute lymphoblastic leukemia and other types of cancer. Acute lymphoblastic leukemia, acute myeloid leukemia, and pancreatic cancer cells are particularly sensitive to combined treatment with L-asparaginase and GCN2 inhibitors. Previously reported studies demonstrated robust antitumor activities of combined treatment with ASNase and GCN2 inhibitors in acute lymphoblastic leukemia, acute myeloid leukemia, and pancreatic cancer cells compared with the results of single-agent L-asparaginase or GCN2 inhibitor treatment. Thus, GCN2 inhibitors may represent sensitizing agents to L-asparaginase used for treating these tumors. In summary, GCN2 inhibition enhances the sensitivity to L-asparaginase treatment by preventing ASNS induction in cancer cells with low ASNS expression at basal levels.

Inhibition of GCN2 may also be an effective strategy for targeting the tumor microenvironment, including the immune system, including tryptophan-dependent immunosurveillance of tumor cells.

The tumor microenvironment [TME; a series of extracellular components and stromal cells (endothelial cells, cancer-associated fibroblasts, tumor-associated macrophages, tumor-infiltrating T cells) that surround the tumor cells] is characterized by deficiencies in oxygen and key nutrients, such as glucose and amino acids, resulting in an overall immune suppressive environment.

Many tumors evolved to escape immune surveillance by taking advantage of their metabolic flexibility and redirecting nutrients for their own advantage. Stromal cells and myeloid-derived suppressor cells (MDSC) within the tumor create a nutrient-poor environment that inhibits immune function and supports tumor growth.

Elevated catabolism of tryptophan, one of the essential amino acids, driven by overexpression of critical enzymes in tryptophan metabolism [Indoleamine-2,3-dioxygenase (IDO) and tryptophan-2,3-dioxygenase (TDO)] is driven by cells of the tumor microenvironment, leading to an immunosuppressive microenvironment in many types of cancer. Local tryptophan depletion is considered to be a crucial T-cell immunosuppressive mechanism. In T cells, the GCN2 kinase has been identified as a molecular sensor of tryptophan deprivation. GCN2 activation by tryptophan depletion induces apoptosis and mitigates T cell proliferation. GCN2 is a key effector signaling component for IDO/TDO and is considered as a metabolic checkpoint of highly tryptophan-dependent T-cells.

The GCN2 pathway is not only important for tumoral immune escape but also plays an active role in modulating other aspects of the tumor microenvironment. GCN2 knockdown has been demonstrated to prevent amino acid deprivation (AAD)-induced expression of Vascular Endothelial Growth Factor (VEGF) which tumors use to enhance nutrient supply via increased vascularization. Thus, activation of the GCN2/ATF4 pathway promotes tumor growth and angiogenesis through AAD-mediated VEGF expression. Abrogation of ATF4 or GCN2 expression significantly inhibited tumor growth in vivo.

Therefore, selective inhibition of GCN2 can both increase the activity of the immune system and decrease vascularization in the tumor microenvironment. The GCN2-eIF2α-ATF4 pathway is critical for maintaining metabolic homeostasis in tumor cells under conditions of stress, and for maintaining an immunosuppressed immune cell microenvironment. The PERK-ATF4 pathway is also critical for maintaining homeostasis in tumors cells under conditions of stress. It has been reported that there is cross talk regulation of both the GCN2 and PERK signaling pathways, such that inhibition of GCN2 can activate PERK as a compensatory mechanism, and vice versa, that inhibition of PERK can activate GCN2 as a compensatory mechanism.

There is a need for modulators of GCN2 and/or PERK that modulate the pro-tumoral aspects of GCN2 and/or PERK, both in the tumor cell (tumor cell autonomous) and in the tumor immune cell microenvironment.

### SUMMARY

Described herein are compounds that modulate (e.g., inhibit or activate) the GCN2 (general control nonderepressible 2) kinase and/or PERK (PKR-like ER kinase) kinase and methods of use thereof for the treatment of disorders, including GCN2 or PERK associated diseases.

In one embodiment, described herein is a compound represented by Formula I-A:

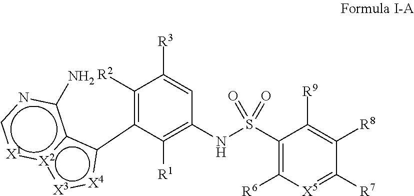

or a pharmaceutically acceptable salt, enantiomer, stereoisomer, or tautomer thereof, wherein: X¹ and X⁴ are each independently selected from the group consisting of CH and N; X² is selected from group consisting of C and N; X³ is selected from the group consisting of CR⁴ and NR⁴; provided that not more than two of X¹, X², X³, and X⁴ is N; X⁵ is selected from the group consisting of CR⁵ and N; R¹ and R² are each independently selected from the group consisting of H, alkoxy, alkyl, cyano, haloalkoxy, haloalkyl, and halogen; R³ is selected from the group consisting of H, alkyl, alkoxy, cyano, and halogen; R⁴ is selected from the group consisting of H, alkyl, alkenyl, alkenylalkyl, alkynyl, alkynylalkyl, cycloalkyl, cycloalkylalkyl, alkoxyalkyl, cycloalkenyl, alkylamino, amide, thioalkyl, heterocyclyl, heterocyclylalkyl, aryl, heteroaryl, and heteroarylalkyl; R⁵ is selected from the group consisting of H, hydroxyalkyl, hydroxycycloalkyl, alkoxyalkyl, alkoxycycloalkyl, halogen, fluoroalkyl, cyano, alkoxy, amine, aminoalkyl, aminocycloalkyl, aminocarbonyl, acylamino, acyloxyalkyl, hydroxyimino, alkoxyimino, alkylamino, cyanoalkyl, alkyl, cycloalkyl, cycloalkoxy, cycloalkylamino, alkoxycarbonyl, and heterocyclylalkyl; R⁶, R⁷, and R⁸ are each independently selected from the group consisting of H, alkyl, alkoxy, hydroxy, cyano, halogen, alkylamino, cycloalkoxy, amine, halogen, haloalkoxy, haloalkyl, amide, alkoxycarbonyl, and hydroxyalkyl; and R⁹ is selected from the group consisting of H, halogen, and alkyl; with the proviso that: i) when X² is C,

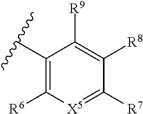

is not

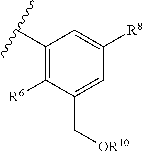

wherein R⁶ is selected from the group consisting of halogen, alkoxy and alkyl; R⁸ is selected from the group consisting of H, halogen, and alkyl; and R¹⁰ is selected from the group consisting of H, alkyl, and acyl; ii) when X² is C and X³ is NR⁴,

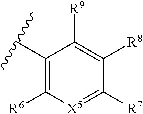

is not

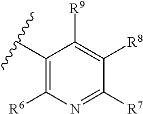

wherein R⁶ is H; R⁷ is selected from the group consisting of H, Cl, and OCH₃; R⁸ is H or Br; and R⁹ is H; and iii) when X² is C and X³ is NR⁴,

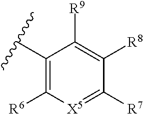

is not

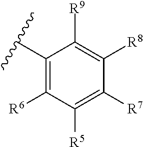

wherein R⁵ is selected from the group consisting of H, F, Cl, CH₃, OCH₃, CF₃, and CN; R⁶ is H or F; R⁷ is selected from the group consisting of H, F, Cl, Br, I, CH₃, OCH₃, OCH₂CH₃, OCH(CH₃)₂, CF₃, OH, and OCF₃; R⁸ is selected from the group consisting of H, F, Cl, CH₃, OCH₃, CF₃, and CN; and R⁹ is H or F.

In another embodiment, described herein is a pharmaceutical composition comprising a compound described herein (e.g., a compound of Formulae I-A, I-B, I-C, I-D, I-E, I-F, and I-G described herein), or a pharmaceutically acceptable salt, enantiomer, stereoisomer, or tautomer thereof, and a pharmaceutically acceptable carrier or excipient.

In another embodiment, described herein is a method of treating a disease caused by a dysregulation of an integrated stress response in a patient in need thereof, comprising administering to the patient a therapeutically effective amount of a compound described herein (e.g., a compound of Formulae I-A, I-B, I-C, I-D, I-E, I-F, and I-G described herein), or a pharmaceutically acceptable salt thereof, or of a pharmaceutical composition described herein.

In another embodiment, described herein is a method of treating a disease caused by a dysregulation of an integrated stress response and/or an unfolded protein response in a patient in need thereof, comprising administering to the patient a therapeutically effective amount of a compound described herein (e.g., a compound of Formulae I-A, I-B, I-C, I-D, I-E, I-F, and I-G described herein), or a pharmaceutically acceptable salt thereof, or of a pharmaceutical composition described herein.

In another embodiment, described herein is a method of modulating the activity of GCN2 kinase in a patient in need thereof, comprising administering to the patient a therapeutically effective amount of a compound described herein (e.g., a compound of Formulae I-A, I-B, I-C, I-D, I-E, I-F, and I-G described herein), or a pharmaceutically acceptable salt thereof, or of a pharmaceutical composition described herein.

In another embodiment, described herein is a method of activating GCN2 kinase in a patient in need thereof, comprising administering to the patient a therapeutically effective amount of a compound described herein (e.g., a compound of Formulae I-A, I-B, I-C, I-D, I-E, I-F, and I-G described herein), or a pharmaceutically acceptable salt thereof, or of a pharmaceutical composition described herein.

In another embodiment, described herein is a method of modulating the activity of PERK kinase in a patient in need thereof, comprising administering to the patient a therapeutically effective amount of a compound described herein (e.g., a compound of Formulae I-A, I-B, I-C, I-D, I-E, I-F, and I-G described herein), or a pharmaceutically acceptable salt thereof, or of a pharmaceutical composition described herein.

In another embodiment, described herein is a method of activating PERK kinase in a patient in need thereof, comprising administering to the patient a therapeutically effective amount of a compound described herein (e.g., a compound of Formulae I-A, I-B, I-C, I-D, I-E, I-F, and I-G described herein), or a pharmaceutically acceptable salt thereof, or of a pharmaceutical composition described herein.

In another embodiment, described herein is a method of inhibiting GCN2 kinase and inhibiting PERK kinase in a patient in need thereof, comprising administering to the patient a therapeutically effective amount of a compound described herein (e.g., a compound of Formulae I-A, I-B, I-C, I-D, I-E, I-F, and I-G described herein), or a pharmaceutically acceptable salt thereof, or of a pharmaceutical composition described herein.

In another embodiment, described herein is a method of inhibiting the activity of GCN2 kinase in a patient in need thereof, comprising administering to the patient a therapeutically effective amount of a compound described herein (e.g., a compound of Formulae I-A, I-B, I-C, I-D, I-E, I-F, and I-G described herein), or a pharmaceutically acceptable salt thereof, or of a pharmaceutical composition described herein.

In another embodiment, described herein is a method of inhibiting the activity of PERK kinase in a patient in need thereof, comprising administering to the patient a therapeutically effective amount of a compound described herein (e.g., a compound of Formulae I-A, I-B, I-C, I-D, I-E, I-F, and I-G described herein), or a pharmaceutically acceptable salt thereof, or of a pharmaceutical composition described herein.

In another embodiment, described herein is a method of treating a disease selected from a GCN2 associated disease and a PERK associated disease, in a patient in need thereof, comprising administering to the patient a therapeutically effective amount of a compound described herein (e.g., a compound of Formulae I-A, I-B, I-C, I-D, I-E, I-F, and I-G described herein), or a pharmaceutically acceptable salt thereof, or of a pharmaceutical composition described herein.

In another embodiment, described herein is a method of treating a disease selected from a GCN2 associated disease and a PERK associated disease, in a patient in need thereof, comprising administering to the patient a therapeutically effective amount of a compound described herein (e.g., a compound of Formulae I-A, I-B, I-C, I-D, I-E, I-F, and I-G described herein), or a pharmaceutically acceptable salt thereof, and a therapeutically effective amount of one or more therapeutic agents.

In another embodiment, described herein is a method of treating a cancer in a patient in need thereof, comprising administering to the patient a therapeutically effective amount of a compound described herein (e.g., a compound of Formulae I-A, I-B, I-C, I-D, I-E, I-F, and I-G described herein) or a pharmaceutically acceptable salt thereof, or of a pharmaceutical composition described herein.

In another embodiment, described herein is a method of treating a disorder selected from the group consisting of melanoma, fibrosarcoma, thyroid cancer, ovarian cancer, colon cancer, pancreatic cancer, lung cancer, bladder cancer, gastrointestinal stromal tumors, solid tumors, blood-borne cancers, acute myelogenous leukemia (AMIL), acute lymphoblastic leukemia (ALL) and other cancers caused by activation of the GCN2 signaling pathway in a patient in need thereof, comprising administering to the patient a therapeutically effective amount of a compound described herein (e.g., a compound of Formulae I-A, I-B, I-C, I-D, I-E, I-F, and I-G described herein) or a pharmaceutically acceptable salt thereof, or of a pharmaceutical composition described herein.

### DETAILED DESCRIPTION

The features and other details of the disclosure are more particularly described below. Certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Definitions

The definitions set forth in this application are intended to clarify terms used throughout this application.

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated in order to facilitate the understanding of the present disclosure.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents, positions of substituents and/or variables are permissible if such combinations result in stable compounds.

As used herein, the singular forms ““a”, “an” and “the” encompass plural references unless the context clearly indicates otherwise.

As used herein, the term “herein” means the entire application.

As used herein, “deuterated” mean that at least one hydrogen atom is replaced by deuterium. In any sample of a deuterated compound, some discrete molecules of the compound will likely have hydrogen, rather than deuterium, at the specified position. However, the percent of molecules of the deuterated compound which have deuterium at the specified position will be much greater than would naturally occur. The deuterium at the deuterated position is enriched.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the disclosed compounds can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure result.

As used herein, the term “optionally substituted” refers to the replacement of one to six hydrogen atoms in a given structure with the radical of a specified substituent including, but not limited to: hydroxy, hydroxyalkyl, alkoxy, halogen, alkyl, aryl, cycloalkyl, heterocyclyl, amino, aminoalkyl, cyano, haloalkyl, haloalkoxy, —OC(═O)—CH₂—Oalkyl. Preferably, “optionally substituted” refers to the replacement of one to four hydrogen atoms in a given structure with the substituents mentioned above. More preferably, one to three hydrogen atoms are replaced by the substituents as mentioned above. It is understood that the substituent can be further substituted.

As used herein, the term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this application, the heteroatoms such as nitrogen may have hydrogen substituents and/or any permissible substituents of organic compounds described herein which satisfy the valences of the heteroatoms.

Substituents can include any substituents described herein, for example, such substituents, if not otherwise specified, can include, for example, a halogen, a hydroxy, a carbonyl (such as a carboxyl, an alkoxycarbonyl, a formyl, or an acyl), an alkoxy, an amino, an amido, an imine, a cyano, a sulfonyl, a heterocyclyl, an aralkyl, a heteroaralkyl, or an aromatic or heteroaromatic moiety. It will be understood by those skilled in the art that substituents can themselves be substituted, if appropriate. For instance, the substituents of a substituted alkyl may include substituted and unsubstituted forms of amino, amido, sulfonyl and as well as ethers, carbonyls (including carboxylates, and esters), —CF₃, —CN and the like. Unless specifically stated as “unsubstituted,” references to chemical moieties herein are understood to include substituted variants. For example, reference to an “aryl” group or moiety implicitly includes both substituted and unsubstituted variants.

As used herein, the term “alkyl” refers to a straight chained or branched non-aromatic hydrocarbon which is completely saturated. Typically, a straight chained or branched alkyl group has from 1 to about 20 carbon atoms, preferably from 1 to about 10, e.g., may be C₁-C₁₀alkyl or e.g., C₁-C₆alkyl unless otherwise defined. Examples of straight chained and branched alkyl groups include, but are not limited to, methyl, ethyl, 1-propyl (n-propyl), 2-propyl, n-butyl, sec-butyl, tertbutyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. Moreover, the term “alkyl” used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. The “alkyl” group may be optionally substituted.

The term “Cₓ-C_{y}” when used in conjunction with a chemical moiety, such as, acyl, acyloxy, alkyl, alkenyl, alkynyl, or alkoxy is meant to include groups that contain from x to y carbons in the chain. For example, the term “Cₓ-C_{y}” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc. C₀ alkyl indicates a hydrogen where the group is in a terminal position, a bond if internal.

The term “alkenyl” as used herein refers to an unsaturated straight or branched hydrocarbon having at least one carbon-carbon double bond. Exemplary alkenyl groups include, but are not limited to, a straight or branched group of 2-6 or 3-4 carbon atoms, referred to herein as C₂-C₆alkenyl, and C₃-C₄alkenyl, respectively. Exemplary alkenyl groups include, but are not limited to, vinyl, allyl, butenyl, pentenyl, etc.

The term “alkynyl” as used herein refers to an unsaturated straight or branched hydrocarbon having at least one carbon-carbon triple bond. Exemplary alkynyl groups include, but are not limited to, straight or branched groups of 2-6, or 3-6 carbon atoms, referred to herein as C₂-C₆alkynyl, and C₃-C₆alkynyl, respectively. Exemplary alkynyl groups include, but are not limited to, ethynyl, propynyl, butynyl, pentynyl, hexynyl, methylpropynyl, etc.

As used herein, the term “alkoxy” refers to a straight or branched, saturated aliphatic (alkyl) hydrocarbon radical bonded to an oxygen atom that is attached to a core structure. Preferably, alkoxy groups have one to six carbon atoms, i.e., may be C₁-C₆ alkoxy. Examples of alkoxy groups include but are not limited to methoxy, ethoxy, propoxy, isopropoxy, butoxy, isobutoxy, tert-butoxy, pentoxy, 3-methyl butoxy and the like.

As used herein, the term “alkoxyalkyl” refers to an alkyl group (as defined above) substituted with an alkoxy group and may be represented by the general formula alkyl-O-alkyl. Examples of alkoxyalkyl groups include but are not limited to methyl-O-ethylene-, ethyl-O-ethylene-.

As used herein, the term “haloalkyl” refers to alkyl group (as defined above) is substituted with one or more halogens. A monohaloalkyl radical, for example, may have a chlorine, bromine, iodine or fluorine atom. Dihalo and polyhaloalkyl radicals may have two or more of the same or different halogen atoms. Examples of haloalkyl include, but are not limited to, chloromethyl, dichloromethyl, trichloromethyl, dichloroethyl, dichloropropyl, fluoromethyl, difluoromethyl, trifluoromethyl, pentafluoroethyl, heptafluoropropyl, difluorochloromethyl, dichlorofluoromethyl, difluoroethyl, difluoropropyl and the like.

As used herein, the term “haloalkoxy” refers to radicals wherein one or more of the hydrogen atoms of the alkoxy group are substituted with one or more halogens. Representative examples of “haloalkoxy” groups include, but not limited to, difluoromethoxy (—OCHF₂), trifluoromethoxy (—OCF₃) or trifluoroethoxy (—OCH₂CF₃).

As used herein, the term “aryl” includes substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings (fused rings) wherein at least one of the rings is aromatic. e.g., the other cyclic rings can be cycloalkyls, cycloalkenyls, cycloalkynyls, aryls, heteroaryls, and/or heterocyclyls. The term “fused” means that the second ring is attached or formed by having two adjacent atoms in common with the first ring. The term “fused” is equivalent to the term “condensed”. Examples of aryl groups include but are not limited to phenyl, naphthyl, phenanthryl, phenol, aniline, indanyl, dihydrobenzofuranyl, dihydroisobenzofuranyl, indolinyl, isoindolinyl, and the like. Unless otherwise specified, aryl groups described herein may be optionally substituted.

As used herein, the terms “polycyclyl”, “polycycle”, and “polycyclic” refer to two or more rings (e.g., cycloalkyls, cycloalkenyls, cycloalkynyls, aryls, heteroaryls, and/or heterocyclyls) in which one or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

As used herein, the term “acyl” refers to a group —C(═O)—R^{w} wherein R^{w} is optionally substituted alkyl. Examples of “acyl” include, but are not limited to, instances where R^{w} is C₁-C₁₀alkyl (C₁-C₁₀acyl) or C₁-C₆-alkyl (C₁-C₆acyl). In some embodiments, each occurrence of the optionally substituted substituent is independently selected from the group consisting of H, OH, alkoxy, cyano, F, and amino. Additional examples of “acyl” include —C(═O)—CH₃, —C(═O)—CH₂—CH₃, —C(═O)—CH₂—CH₂—CH₃, or —C(═O)—CH(CH₃)₂.

As used herein, the terms “amine” and “amino” refer to both unsubstituted and substituted amines and salts thereof, e.g., a moiety that can be represented by:

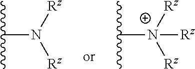

wherein each R^{z} independently represents hydrogen or a hydrocarbyl group, or R^{z} groups are taken together with the N atom to which they are attached complete a heterocycle having from 4 to 8 atoms in the ring structure.

As used herein, the terms “amide” and “amido” refers to a group represented by

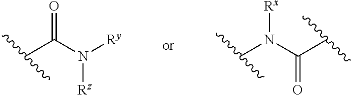

wherein R^{x}, R^{y}, and R^{z} each independently represents hydrogen or a hydrocarbyl group, or R^{y} and R^{z} are taken together with the N atom to which they are attached complete a heterocyclyl having from 4 to 8 atoms in the ring structure.

As used herein, the term “acylamino” refers to an amino group, as defined above, substituted with an acyl group.

As used herein, the term “aminocarbonyl” refers to a carbonyl group substituted with an amino group.

As used herein, the term “alkenylalkyl” refers to an alkyl group substituted with an alkenyl group.

As used herein, the term “alkynylalkyl” refers to an alkyl group substituted with an alkynyl group.

As used herein, the term “alkylamino” refers to an amino group, as defined above, substituted with at least one alkyl group.

As used herein, the term “aminoalkyl” refers to an alkyl group substituted with an amino group.

As used herein, the term “amidoalkyl” refers to an alkyl group substituted with an amido group.

As used herein, the term “cyanoalkyl” refers to an alkyl group substituted with a cyano group.

As used herein, the term “cycloalkoxyalkyl” refers to an alkyl group (as defined above) substituted with a cycloalkoxy group and may be represented by the general formula cycloalkyl-O-alkyl. Examples of cycloalkoxyalkyl groups include but are not limited to cyclopropyl-O-methylene-, cyclopropyl-O-ethylene.

As used herein, the term “cycloalkylalkyl” refers to an alkyl group substituted with a cycloalkyl group.

As used herein, the term “heteroarylalkyl” refers to an alkyl group substituted with heteroaryl group.

As used herein, the term “heterocyclylalkyl” refers to an alkyl group substituted with a heterocyclyl group.

As used herein, the term “hydroxyalkyl” refers to an alkyl group substituted with a hydroxy group.

As used herein, the term “cycloalkyl” alone or in combination with other term(s) refers to a cyclic hydrocarbon which is completely saturated. “Cycloalkyl” includes monocyclic, bicyclic, and tricyclic rings. Typically, a monocyclic cycloalkyl group has from 3 to about 10 carbon atoms, more typically 3 to 8 carbon atoms (e.g., C₃-C₁₀cycloalkyl or e.g., C₃-C₆cycloalkyl unless otherwise defined. Examples of monocyclic cycloalkyls include cyclopropyl, cyclobutyl, cyclopentyl, cyclohexyl, cycloheptyl and the like. The second ring of a bicyclic cycloalkyl or, the second or third rings of a tricyclic cycloalkyl, may be selected from saturated, unsaturated and aromatic rings. Cycloalkyl includes bicyclic and tricyclic molecules in which one, two or three or more atoms are shared between the two rings. Cycloalkyls can be further substituted with alkyls, alkenyls, alkoxys, aminoalkyls, carbonyl-substituted alkyls, —CF₃, —CN, and the like.

As used herein, the term “cycloalkylalkyl” refers to an alkyl group substituted with a cycloalkyl group.

As used herein, the term “cyano” refers to —CN group.

As used herein, the term “hydroxy” or “hydroxyl” refers to —OH group.

As used herein, the term “halo” or “halogen” alone or in combination with other term(s) means chloro, fluoro, bromo, and iodo.

As used herein, the term “heteroatom” refers an atom of any element other than carbon or hydrogen. Exemplary heteroatoms are nitrogen (N), oxygen (O), sulfur (S), and silicon (Si).

As used herein, the terms “heterocyclyl”, “heterocycloalkyl”, “heterocycle”, and “heterocyclic” refer to a non-aromatic, saturated or partially saturated, including monocyclic, polycyclic (e.g., bicyclic, tricyclic) bridged, or fused, ring system of 3 to 15 member having at least one heteroatom or heterogroup selected from O, N, S, S(O), S(O)₂, NH or C(O) with the remaining ring atoms being independently selected from the group consisting of carbon, oxygen, nitrogen, and sulfur. Examples of “heterocycloalkyl” include, but are not limited to azetidinyl, oxetanyl, imidazolidinyl, pyrrolidinyl, oxazolidinyl, thiazolidinyl, pyrazolidinyl, tetrahydrofuranyl, piperidinyl, piperazinyl, tetrahydropyranyl, morpholinyl, thiomorpholinyl, 1,4-dioxanyl, dioxidothiomorpholinyl, oxapiperazinyl, oxapiperidinyl, tetrahydrofuryl, tetrahydropyranyl, tetrahydrothiophenyl, dihydropyranyl, indolinyl, indolinylmethyl, 2-azabicyclo[2.2.2]octanyl, azocinyl, chromanyl, xanthenyl and N-oxides thereof. Attachment of a heterocycloalkyl substituent can occur via either a carbon atom or a heteroatom. A heterocycloalkyl group can be optionally substituted with one or more suitable groups by one or more aforesaid groups. Preferably “heterocycloalkyl” refers to 5- to 6-membered ring selected from the group consisting of azetidinyl, oxetanyl, imidazolidinyl, pyrrolidinyl, oxazolidinyl, thiazolidinyl, pyrazolidinyl, tetrahydrofuranyl, piperidinyl, piperazinyl, tetrahydropyranyl, morpholinyl, thiomorpholinyl, 1,4-dioxanyl and N-oxides thereof. More preferably, “heterocycloalkyl” includes azetidinyl, pyrrolidinyl, morpholinyl and piperidinyl. Heterocycloalkyl are optionally substituted by one or more aforesaid groups.

As used herein, the term “heteroaryl” refers to substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The term “heteroaryl” also refers to substituted or unsubstituted aromatic or partly aromatic ring systems containing at least one heteroatom and having two or more cyclic rings (bicyclic, tricyclic, or polycyclic), containing 8 to 20 ring atoms, suitably 5 to 10 ring atoms, which may be linked covalently, or fused in which two or more atoms are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. The rings may contain an N or S atom, wherein the N or S atom is optionally oxidized, or the N atom is optionally quaternized. All heteroaryls are optionally substituted. Any suitable ring position of the heteroaryl moiety may be covalently linked to a defined chemical structure. Examples of heteroaryl include, but are not limited to: furanyl, thienyl, pyrrolyl, pyrazolyl, imidazolyl, oxazolyl, cinnolinyl, isoxazolyl, thiazolyl, isothiazolyl, 1H-tetrazolyl, oxadiazolyl, thiadiazolyl, triazolyl, pyridyl, pyrimidinyl, pyrazinyl, pyridazinyl, triazinyl, benzoxazolyl, benzisoxazolyl, benzothiazolyl, benzofuranyl, benzothienyl, benzotriazinyl, phthalazinyl, thianthrene, dibenzofuranyl, dibenzothienyl, benzimidazolyl, indolyl, isoindolyl, indazolyl, quinolinyl, isoquinolinyl, quinazolinyl, quinoxalinyl, purinyl, pteridinyl, 9H-carbazolyl, alpha-carboline, indolizinyl, benzoisothiazolyl, benzoxazolyl, pyrrolopyridyl, furopyridinyl, purinyl, benzothiadiazolyl, benzoxadiazolyl, benzotriazolyl, benzotriadiazolyl, carbazolyl, dibenzothienyl, acridinyl and the like.

As used herein, the term “hydrocarbyl” refers to a group that is bonded through a carbon atom that does not have a ═O or ═S substituent, and typically has at least one carbon-hydrogen bond and a primarily carbon backbone but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, cycloalkyl, heterocyclyl, alkyl, alkenyl, alkynyl, and combinations thereof.

As used herein, the term “sulfonamide” is represented by:

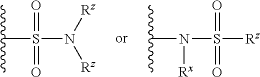

wherein R^{z}, at each occurrence, independently represents a hydrogen, alkyl or cycloalkyl group, or R^{z} groups taken together with the N atom to which they are attached complete a heterocycle having from 4 to 8 atoms in the ring structure.

As used herein, the term “acyloxy” refers to the moiety represented by:

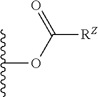

wherein R^{z} represents a hydrocarbyl group.

As used herein, the terms “sulfonyl” refers to the group —S(O)₂—R^{6d} wherein R^{6d} represents alkyl or cycloalkyl.

A “combination therapy” is a treatment that includes the administration of two or more therapeutic agents, e.g., a compound of Formula I-A, I-B, I-C, I-D, I-E, I-F, or I-G and the enzyme asparaginase (ASNase) or a derivative thereof, to a patient in need thereof. [00082]“Disease,” “disorder,” and “condition” are used interchangeably herein. [00083]“Individual,” “patient,” or “subject” are used interchangeably and include any animal, including mammals, preferably mice, rats, other rodents, rabbits, dogs, cats, swine, cattle, sheep, horses, or primates, and most preferably humans. The compounds described herein can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like).

The compounds described herein are useful for the treatment of diseases driven by GCN2 (sometimes to be abbreviated as “GCN2 associated disease” in the present specification), for example, cancer [e.g., colorectal cancer (e.g., colorectal cancer, rectal cancer, anal cancer, familial colorectal cancer, hereditary nonpolyposis colorectal cancer, gastrointestinal stromal tumor), lung cancer (e.g., non-small cell lung cancer, small cell lung cancer, malignant mesothelioma), mesothelioma, pancreatic cancer (e.g., pancreatic duct cancer, pancreatic endocrine tumor), pharyngeal cancer, laryngeal cancer, esophagus cancer, gastric cancer (e.g., papillary adenocarcinoma, mucinous adenocarcinoma, adenosquamous carcinoma), duodenal cancer, small intestinal cancer, breast cancer (e.g., invasive ductal carcinoma, ductal carcinoma in situ, inflammatory breast cancer), ovarian cancer (e.g., ovarian epithelial carcinoma, extragonadal germ cell tumor, ovarian germ cell tumor, ovarian low malignant potential tumor), testis tumor, prostate cancer (e.g., hormone-dependent prostate cancer, non-hormone dependent prostate cancer, castration-resistant prostate cancer), liver cancer (e.g., hepatoma, primary liver cancer, extrahepatic bile duct cancer), thyroid cancer (e.g., medullary thyroid carcinoma), renal cancer (e.g., renal cell carcinoma (e.g., clear cell renal cell carcinoma), transitional cell carcinoma of renal pelvis and ureter), uterine cancer (e.g., cervical cancer, uterine body cancer, uterus sarcoma), gestational choriocarcinoma, brain tumor (e.g., medulloblastoma, glioma, pineal astrocytoma, pilocytic astrocytoma, diffuse astrocytoma, anaplastic astrocytoma, hypophyseal adenoma), retina blastoma, skin cancer (e.g., basal cell carcinoma, malignant melanoma (melanoma)), sarcoma (e.g., rhabdomyosarcoma, leiomyosarcoma, soft tissue sarcoma, spindle cell sarcoma, osteosarcoma), malignant bone tumor, urinary bladder cancer, hematologic cancer (e.g., multiple myeloma, leukemia (e.g., acute myeloid leukemia, acute lymphocytic leukemia (including blast crisis of chronic leukemia)), malignant lymphoma, Hodgkin's disease, chronic myeloproliferative disease), cancer of unknown primary nucleus], cancer growth inhibitor, cancer metastasis inhibitor, apoptosis promoter, and for the prophylaxis or treatment of precancerous lesion (e.g., bone marrow myelodysplastic syndrome).

The compounds described herein, e.g., a compound of Formula I-A, I-B, I-C, I-D, I-E, I-F, or I-G as defined herein, may be used in combination with one or more additional therapeutic agents to treat a disorder described herein, such as a cancer described herein. In some embodiments, the compounds described herein may be used in combination with hormonal therapeutic agents, chemotherapeutic agents, immunotherapeutic agents, medicaments inhibiting actions of cell growth factor and receptor thereof, such as PERK inhibitors and autophagy inhibitors, the enzyme asparaginase (ASNase), and the like.

“Pharmaceutically or pharmacologically acceptable” include molecular entities and compositions that do not produce an adverse, allergic or other untoward reaction when administered to an animal, or a human, as appropriate. For human administration, preparations should meet sterility, pyrogenicity, and general safety and purity standards as required by FDA Office of Biologics standards.

The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable carriers.

The term “pharmaceutically acceptable salt(s)” as used herein refers to salts of acidic or basic groups that may be present in compounds used in the compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including, but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that are acidic in nature are capable of forming base salts with various pharmacologically acceptable cations. Examples of such salts include alkali metal or alkaline earth metal salts, particularly calcium, magnesium, sodium, lithium, zinc, potassium, and iron salts. Compounds included in the present compositions that include a basic or acidic moiety may also form pharmaceutically acceptable salts with various amino acids. The compounds of the disclosure may contain both acidic and basic groups; for example, one amino and one carboxylic acid group. In such a case, the compound can exist as an acid addition salt, a zwitterion, or a base salt.

The compounds of the disclosure may contain one or more chiral centers and, therefore, exist as stereoisomers. The term “stereoisomers” when used herein consist of all enantiomers or diastereomers. These compounds may be designated by the symbol “R” or “S” depending on the configuration of substituents around the stereogenic carbon atom, but the skilled artisan will recognize that a structure may denote a chiral center implicitly. These compounds may also be designated by “(+)” and “(−)” based on their optical rotation properties. The presently described compounds encompasses various stereoisomers of these compounds and mixtures thereof. Mixtures of enantiomers or diastereomers may be designated by the symbol “(±)” in nomenclature, but the skilled artisan will recognize that a structure may denote a chiral center implicitly.

In the present specification, the term “therapeutically effective amount” means the amount of the subject compound that will elicit the biological or medical response of a tissue, system or animal, (e.g., mammal or human) that is being sought by the researcher, veterinarian, medical doctor or other clinician. The compounds described herein are administered in therapeutically effective amounts to treat a disorder. [00092]“Treating” includes any effect, e.g., lessening, reducing, modulating, or eliminating, that results in the improvement of the condition, disease, disorder, and the like.

The disclosure also embraces isotopically labeled compounds which are identical to those recited herein, except that one or more atoms are replaced by an atom having an atomic mass or mass number different from the atomic mass or mass number usually found in nature. Examples of isotopes that can be incorporated into the disclosed compounds include isotopes of hydrogen, carbon, nitrogen, oxygen, phosphorus, sulfur, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. For example, a compound of the disclosure may have one or more H atom replaced with deuterium.

Individual enantiomers and diastereomers of the disclosed compounds can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns or (4) kinetic resolution using stereoselective chemical or enzymatic reagents. Racemic mixtures can also be resolved into their component enantiomers by well-known methods, such as chiral-phase liquid chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantio- and diastereoselective transformations and may involve the use of chiral auxiliaries. For examples, see Carreira and Kvaerno, Classics in Stereoselective Synthesis, Wiley-VCH: Weinheim, 2009.

### Compounds

In one embodiment, described herein is a compound represented by Formula I-A:

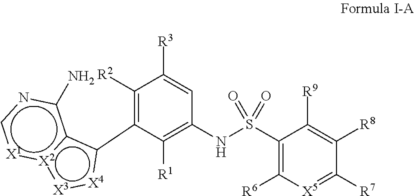

or a pharmaceutically acceptable salt, enantiomer, stereoisomer, or tautomer thereof, wherein:
X¹ and X⁴ are each independently selected from the group consisting of CH and N;
X² is selected from group consisting of C and N;
X³ is selected from the group consisting of CR⁴ and N;
provided that not more than two of X¹, X², X³, and X⁴ is N;
X⁵ is selected from the group consisting of CR⁵ and N;
R¹ and R² are each independently selected from the group consisting of H, alkoxy, alkyl, cyano, haloalkoxy, haloalkyl, and halogen;
R³ is selected from the group consisting of H, alkyl, alkoxy, cyano, and halogen;
R⁴ is selected from the group consisting of H, alkyl, alkenyl, alkenylalkyl, alkynyl, alkynylalkyl, cycloalkyl, cycloalkylalkyl, alkoxyalkyl, cycloalkenyl, alkylamino, amide, thioalkyl, heterocyclyl, heterocyclylalkyl, aryl, heteroaryl, and heteroarylalkyl;
R⁵ is selected from the group consisting of H, hydroxyalkyl, hydroxycycloalkyl, alkoxyalkyl, alkoxycycloalkyl, halogen, fluoroalkyl, cyano, alkoxy, amine, aminoalkyl, aminocycloalkyl, aminocarbonyl, acylamino, acyloxyalkyl, hydroxyimino, alkoxyimino, alkylamino, cyanoalkyl, alkyl, cycloalkyl, cycloalkoxy, cycloalkylamino, alkoxycarbonyl, and heterocyclylalkyl;
R⁶, R⁷, and R⁸ are each independently selected from the group consisting of H, alkyl, alkoxy, hydroxy, cyano, halogen, alkylamino, cycloalkoxy, amine, halogen, haloalkoxy, haloalkyl, amide, alkoxycarbonyl, and hydroxyalkyl; and
R⁹ is selected from the group consisting of H, halogen, and alkyl;
with the proviso that:
when X² is C,

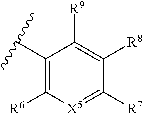

is not

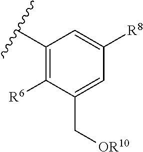

wherein R⁶ is selected from the group consisting of halogen, alkoxy and alkyl;
R⁸ is selected from the group consisting of H, halogen, and alkyl; and
R¹⁰ is selected from the group consisting of H, alkyl, and acyl.

In one embodiment, described herein is a compound represented by Formula I-A:

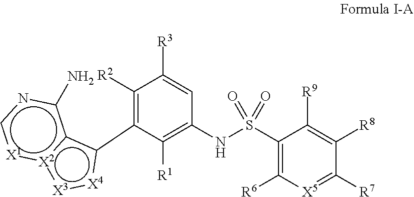

or a pharmaceutically acceptable salt, enantiomer, stereoisomer, or tautomer thereof, wherein:
X¹ and X⁴ are each independently selected from the group consisting of CH and N;
X² is selected from group consisting of C and N;
X³ is selected from the group consisting of CR⁴ and NR⁴;
provided that not more than two of X¹, X², X³, and X⁴ is N;
X⁵ is selected from the group consisting of CR⁵ and N;
R¹ and R² are each independently selected from the group consisting of H, alkoxy, alkyl, cyano, haloalkoxy, haloalkyl, and halogen;
R³ is selected from the group consisting of H, alkyl, alkoxy, cyano, and halogen;
R⁴ is selected from the group consisting of H, alkyl, alkenyl, alkenylalkyl, alkynyl, alkynylalkyl, cycloalkyl, cycloalkylalkyl, alkoxyalkyl, cycloalkenyl, alkylamino, amide, thioalkyl, heterocyclyl, heterocyclylalkyl, aryl, heteroaryl, and heteroarylalkyl;
R⁵ is selected from the group consisting of H, hydroxyalkyl, hydroxycycloalkyl, alkoxyalkyl, alkoxycycloalkyl, halogen, fluoroalkyl, cyano, alkoxy, amine, aminoalkyl, aminocycloalkyl, aminocarbonyl, acylamino, acyloxyalkyl, hydroxyimino, alkoxyimino, alkylamino, cyanoalkyl, alkyl, cycloalkyl, cycloalkoxy, cycloalkylamino, alkoxycarbonyl, and heterocyclylalkyl;
R⁶, R⁷, and R⁸ are each independently selected from the group consisting of H, alkyl, alkoxy, hydroxy, cyano, halogen, alkylamino, cycloalkoxy, amine, halogen, haloalkoxy, haloalkyl, amide, alkoxycarbonyl, and hydroxyalkyl; and
R⁹ is selected from the group consisting of H, halogen, and alkyl;
with the proviso that:
   i) when X² is C,

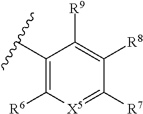

is not

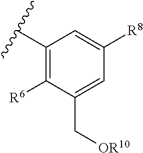

wherein R⁶ is selected from the group consisting of halogen, alkoxy and alkyl;
R⁸ is selected from the group consisting of H, halogen, and alkyl; and
R¹⁰ is selected from the group consisting of H, alkyl, and acyl;
ii) when X² is C and X³ is NR⁴,

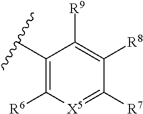

is not

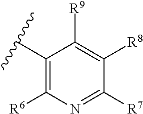

wherein R⁶ is H;
R⁷ is selected from the group consisting of H, Cl, and OCH₃;
R⁸ is H or Br; and
R⁹ is H; and
iii) when X² is C and X³ is NR⁴,

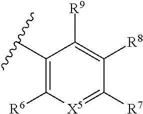

is not

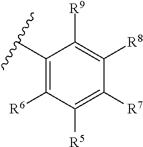

wherein R⁵ is selected from the group consisting of H, F, Cl, CH₃, OCH₃, CF₃, and CN;
R⁶ is H or F;
R⁷ is selected from the group consisting of H, F, Cl, Br, I, CH₃, OCH₃, OCH₂CH₃, OCH(CH₃)₂, CF₃, OH, and OCF₃;
R⁸ is selected from the group consisting of H, F, Cl, CH₃, OCH₃, CF₃, and CN; and
R⁹ is H or F.

In some embodiments, at least one of R¹, R², and R³ is halogen. In some embodiments, at least one of R¹, R², and R³ is fluoro. In some embodiments, R¹ is fluoro

In some embodiments, X¹ is N.

In some embodiments, X² is N.

In some embodiments, X³ is NR⁴.

In some embodiments, X³ is CR⁴.

In another embodiment, described herein is a compound represented by Formula I-B:

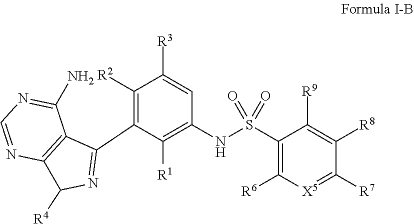

or a pharmaceutically acceptable salt, enantiomer, stereoisomer, or tautomer thereof, wherein:
X⁵ is selected from the group consisting of CR⁵ and N;
R¹, and R² are each independently selected from the group consisting of H, alkoxy, alkyl, cyano, haloalkoxy, haloalkyl, and halogen;
R³ is selected from the group consisting of H, alkyl, and halogen;
R⁴ is selected from the group consisting of H, alkyl, alkenyl, alkenylalkyl, alkynyl, alkynylalkyl, cycloalkyl, cycloalkylalkyl, alkoxyalkyl, cycloalkenyl, alkylamino, amide, thioalkyl, heterocyclyl, heterocyclylalkyl, aryl, heteroaryl, and heteroarylalkyl;
R⁵ is selected from the group consisting of H, hydroxyalkyl, hydroxycycloalkyl, alkoxyalkyl, alkoxycycloalkyl, halogen, fluoroalkyl, cyano, alkoxy, amine, aminoalkyl, aminocycloalkyl, aminocarbonyl, acylamino, acyloxyalkyl, hydroxyimino, alkoxyimino, alkylamino, cyanoalkyl, alkyl, cycloalkyl, cycloalkoxy, cycloalkylamino, alkoxycarbonyl, and heterocyclylalkyl;
R⁶, R⁷, and R⁸ are each independently selected from the group consisting of H, alkyl, alkoxy, hydroxy, cyano, halogen, alkylamino, cycloalkoxy, amine, halogen, haloalkoxy, haloalkyl, amide, alkoxycarbonyl, and hydroxyalkyl; and
R⁹ is selected from the group consisting of H, halogen, and alkyl;
with the proviso that:

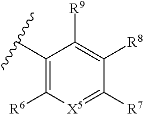

is not

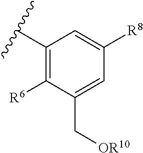

wherein R⁶ is selected from the group consisting of halogen, alkoxy and alkyl;
R⁸ is selected from the group consisting of H, halogen and alkyl; and
R¹⁰ is selected from the group consisting of H, alkyl, and acyl.

In another embodiment, described herein is a compound represented by Formula I-B:

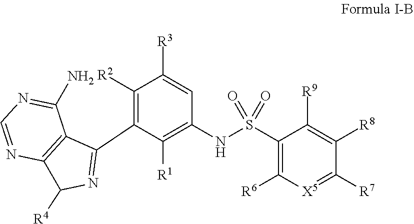

or a pharmaceutically acceptable salt, enantiomer, stereoisomer, or tautomer thereof, wherein:
X⁵ is selected from the group consisting of CR⁵ and N;
R¹ and R² are each independently selected from the group consisting of H, alkoxy, alkyl, cyano, haloalkoxy, haloalkyl, and halogen;
R³ is selected from the group consisting of H, alkyl, and halogen;
R⁴ is selected from the group consisting of H, alkyl, alkenyl, alkenylalkyl, alkynyl, alkynylalkyl, cycloalkyl, cycloalkylalkyl, alkoxyalkyl, cycloalkenyl, alkylamino, amide, thioalkyl, heterocyclyl, heterocyclylalkyl, aryl, heteroaryl, and heteroarylalkyl;
R⁵ is selected from the group consisting of H, hydroxyalkyl, hydroxycycloalkyl, alkoxyalkyl, alkoxycycloalkyl, halogen, fluoroalkyl, cyano, alkoxy, amine, aminoalkyl, aminocycloalkyl, aminocarbonyl, acylamino, acyloxyalkyl, hydroxyimino, alkoxyimino, alkylamino, cyanoalkyl, alkyl, cycloalkyl, cycloalkoxy, cycloalkylamino, alkoxycarbonyl, and heterocyclylalkyl;
R⁶, R⁷, and R⁸ are each independently selected from the group consisting of H, alkyl, alkoxy, hydroxy, cyano, halogen, alkylamino, cycloalkoxy, amine, halogen, haloalkoxy, haloalkyl, amide, alkoxycarbonyl, and hydroxyalkyl; and
R⁹ is selected from the group consisting of H, halogen, and alkyl;
with the proviso that:
   i)

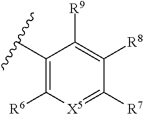

is not

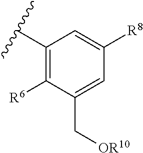

wherein R⁶ is selected from the group consisting of halogen, alkoxy and alkyl;
R⁸ is selected from the group consisting of H, halogen and alkyl; and
R¹⁰ is selected from the group consisting of H, alkyl, and acyl;
ii)

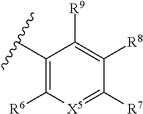

is not

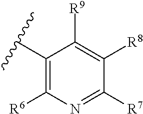

wherein R⁶ is H;
R⁷ is selected from the group consisting of H, Cl, and OCH₃;
R⁸ is H or Br; and
R⁹ is H; and
iii)

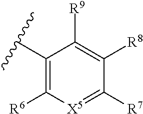

is not

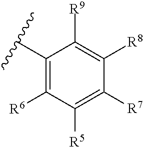

wherein R⁵ is selected from the group consisting of H, F, Cl, CH₃, OCH₃, CF₃, and CN;
R⁶ is H or F;
R⁷ is selected from the group consisting of H, F, Cl, Br, I, CH₃, OCH₃, OCH₂CH₃, OCH(CH₃)₂, CF₃, OH, and OCF₃;
R⁸ is selected from the group consisting of H, F, Cl, CH₃, OCH₃, CF₃, and CN; and
R⁹ is H or F.

In some embodiments, R² is H and R³ is H.

In some embodiments, R¹ is F, R² is H, and R³ is H.

In some embodiments, R² is F and R³ is H.

In some embodiments, R² is H and R³ is F.

In another embodiment, described herein is a compound represented by Formula I-C:

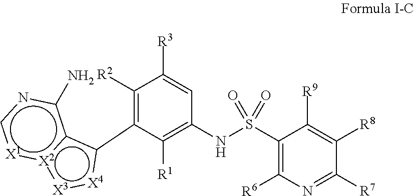

or a pharmaceutically acceptable salt, enantiomer, stereoisomer, or tautomer thereof, wherein:
X¹ and X⁴ are each independently selected from the group consisting of CH and N;
X² is selected from group consisting of C and N;
X³ is selected from the group consisting of CR⁴ and N;
provided that not more than two of X¹, X², X³, and X⁴ is N;
R¹, and R² are each independently selected from the group consisting of H, alkoxy, alkyl, cyano, haloalkoxy, haloalkyl, and halogen;
R³ is selected from the group consisting of H, alkyl, and halogen;
R⁴ is selected from the group consisting of H, alkyl, alkenyl, alkenylalkyl, alkynyl, alkynylalkyl, cycloalkyl, cycloalkylalkyl, alkoxyalkyl, cycloalkenyl, alkylamino, amide, thioalkyl, heterocyclyl, heterocyclylalkyl, aryl, heteroaryl, and heteroarylalkyl;
R⁶, R⁷, and R⁸ are each independently selected from the group consisting of H, alkyl, alkoxy, hydroxy, cyano, halogen, alkylamino, cycloalkoxy, amine, halogen, haloalkoxy, haloalkyl, amide, alkoxycarbonyl, and hydroxyalkyl; and
R⁹ is selected from the group consisting of H, halogen, and alkyl.

In another embodiment, described herein is a compound represented by Formula I-C:

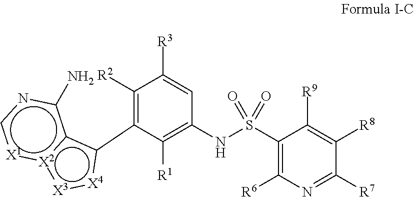

or a pharmaceutically acceptable salt, enantiomer, stereoisomer, or tautomer thereof, wherein:
X¹ and X⁴ are each independently selected from the group consisting of CH and N;
X² is selected from group consisting of C and N;
X³ is selected from the group consisting of CR⁴ and NR⁴;
provided that not more than two of X¹, X², X³, and X⁴ is N;
R¹ and R² are each independently selected from the group consisting of H, alkoxy, alkyl, cyano, haloalkoxy, haloalkyl, and halogen;
R³ is selected from the group consisting of H, alkyl, and halogen;
R⁴ is selected from the group consisting of H, alkyl, alkenyl, alkenylalkyl, alkynyl, alkynylalkyl, cycloalkyl, cycloalkylalkyl, alkoxyalkyl, cycloalkenyl, alkylamino, amide, thioalkyl, heterocyclyl, heterocyclylalkyl, aryl, heteroaryl, and heteroarylalkyl;
R⁶, R⁷, and R⁸ are each independently selected from the group consisting of H, alkyl, alkoxy, hydroxy, cyano, halogen, alkylamino, cycloalkoxy, amine, halogen, haloalkoxy, haloalkyl, amide, alkoxycarbonyl, and hydroxyalkyl; and
R⁹ is selected from the group consisting of H, halogen, and alkyl.
with proviso that
when X² is C, X³ is NR⁴, R⁶ is H, R⁸ is H or Br, and R⁹ is H, R⁷ is not H, Cl, or OCH₃.

In some embodiments, R² is H and R³ is H.

In some embodiments, R¹ is F, R² is H, and R³ is H.

In some embodiments, R² is F and R³ is H.

In some embodiments, R² is H and R³ is F.

In some embodiments, X¹ is N.

In some embodiments, X² is N.

In some embodiments, X³ is NR⁴.

In some embodiments, X³ is CR⁴.

In another embodiment, described herein is a compound represented by Formula I-D:

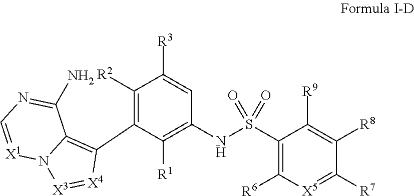

or a pharmaceutically acceptable salt, enantiomer, stereoisomer, or tautomer thereof, wherein:
X¹ and X⁴ are each independently selected from the group consisting of CH and N;
X³ is selected from the group consisting of CR⁴ and N;
provided that not more than one of X¹, X³, and X⁴ is N;
X⁵ is selected from the group consisting of CR⁵ and N;
R¹, and R² are each independently selected from the group consisting of H, alkoxy, alkyl, cyano, haloalkoxy, haloalkyl, and halogen;
R³ is selected from the group consisting of H, alkyl, and halogen;
R⁴ is selected from the group consisting of H, alkyl, alkenyl, alkenylalkyl, alkynyl, alkynylalkyl, cycloalkyl, cycloalkylalkyl, alkoxyalkyl, cycloalkenyl, alkylamino, amide, thioalkyl, heterocyclyl, heterocyclylalkyl, aryl, heteroaryl, and heteroarylalkyl;
R⁵ is selected from the group consisting of H, hydroxyalkyl, hydroxycycloalkyl, alkoxyalkyl, alkoxycycloalkyl, halogen, fluoroalkyl, cyano, alkoxy, amine, aminoalkyl, aminocycloalkyl, aminocarbonyl, acylamino, acyloxyalkyl, hydroxyimino, alkoxyimino, alkylamino, cyanoalkyl, alkyl, cycloalkyl, cycloalkoxy, cycloalkylamino, alkoxycarbonyl, and heterocyclylalkyl;
R⁶, R⁷, and R⁸ are each independently selected from the group consisting of H, alkyl, alkoxy, hydroxy, cyano, halogen, alkylamino, cycloalkoxy, amine, halogen, haloalkoxy, haloalkyl, amide, alkoxycarbonyl, and hydroxyalkyl; and
R⁹ is selected from the group consisting of H, halogen, and alkyl.

In another embodiment, described herein is a compound represented by Formula I-D:

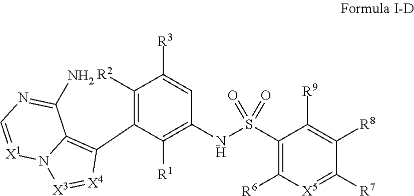

or a pharmaceutically acceptable salt, enantiomer, stereoisomer, or tautomer thereof, wherein:
X¹ and X⁴ are each independently selected from the group consisting of CH and N;
X³ is selected from the group consisting of CR⁴ and NR⁴;
provided that not more than one of X¹, X³, and X⁴ is N;
X⁵ is selected from the group consisting of CR⁵ and N;
R¹ and R² are each independently selected from the group consisting of H, alkoxy, alkyl, cyano, haloalkoxy, haloalkyl, and halogen;
R³ is selected from the group consisting of H, alkyl, and halogen;
R⁴ is selected from the group consisting of H, alkyl, alkenyl, alkenylalkyl, alkynyl, alkynylalkyl, cycloalkyl, cycloalkylalkyl, alkoxyalkyl, cycloalkenyl, alkylamino, amide, thioalkyl, heterocyclyl, heterocyclylalkyl, aryl, heteroaryl, and heteroarylalkyl;
R⁵ is selected from the group consisting of H, hydroxyalkyl, hydroxycycloalkyl, alkoxyalkyl, alkoxycycloalkyl, halogen, fluoroalkyl, cyano, alkoxy, amine, aminoalkyl, aminocycloalkyl, aminocarbonyl, acylamino, acyloxyalkyl, hydroxyimino, alkoxyimino, alkylamino, cyanoalkyl, alkyl, cycloalkyl, cycloalkoxy, cycloalkylamino, alkoxycarbonyl, and heterocyclylalkyl;
R⁶, R⁷, and R⁸ are each independently selected from the group consisting of H, alkyl, alkoxy, hydroxy, cyano, halogen, alkylamino, cycloalkoxy, amine, halogen, haloalkoxy, haloalkyl, amide, alkoxycarbonyl, and hydroxyalkyl; and
R⁹ is selected from the group consisting of H, halogen, and alkyl.

In some embodiments, R² is H and R³ is H.

In some embodiments, R¹ is F, R² is H, and R³ is H.

In some embodiments, R² is F and R³ is H.

In some embodiments, R² is H and R³ is F.

In some embodiments, X¹ is CH, X³ is CR⁴, and X⁴ is N.

In another embodiment, described herein is a compound represented by Formula I-E:

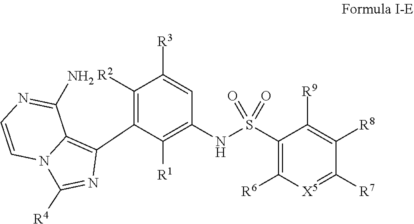

or a pharmaceutically acceptable salt, enantiomer, stereoisomer, or tautomer thereof, wherein:
X⁵ is selected from the group consisting of CR⁵ and N;
R¹, and R² are each independently selected from the group consisting of H, alkoxy, alkyl, cyano, haloalkoxy, haloalkyl, and halogen;
R³ is selected from the group consisting of H, alkyl, and halogen;
R⁴ is selected from the group consisting of H, alkyl, alkenyl, alkenylalkyl, alkynyl, alkynylalkyl, cycloalkyl, cycloalkylalkyl, alkoxyalkyl, cycloalkenyl, alkylamino, amide, thioalkyl, heterocyclyl, heterocyclylalkyl, aryl, heteroaryl, and heteroarylalkyl;
R⁵ is selected from the group consisting of H, hydroxyalkyl, hydroxycycloalkyl, alkoxyalkyl, alkoxycycloalkyl, halogen, fluoroalkyl, cyano, alkoxy, amine, aminoalkyl, aminocycloalkyl, aminocarbonyl, acylamino, acyloxyalkyl, hydroxyimino, alkoxyimino, alkylamino, cyanoalkyl, alkyl, cycloalkyl, cycloalkoxy, cycloalkylamino, alkoxycarbonyl, and heterocyclylalkyl;
R⁶, R⁷, and R⁸ are each independently selected from the group consisting of H, alkyl, alkoxy, hydroxy, cyano, halogen, alkylamino, cycloalkoxy, amine, halogen, haloalkoxy, haloalkyl, amide, alkoxycarbonyl, and hydroxyalkyl; and
R⁹ is selected from the group consisting of H, halogen, and alkyl.

In some embodiments, R² is H and R³ is H.

In some embodiments, R¹ is F, R² is H, and R³ is H.

In some embodiments, R² is F and R³ is H.

In some embodiments, R² is H and R³ is F.

In another embodiment, described herein is a compound represented by Formula I-F:

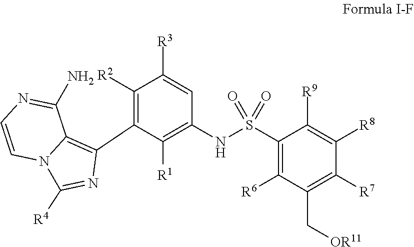

or a pharmaceutically acceptable salt, enantiomer, stereoisomer, or tautomer thereof, wherein:
R¹, and R² are each independently selected from the group consisting of H, alkoxy, alkyl, cyano, haloalkoxy, haloalkyl, and halogen;
R³ is selected from the group consisting of H, alkyl, and halogen;
R⁴ is selected from the group consisting of H, alkyl, alkenyl, alkenylalkyl, alkynyl, alkynylalkyl, cycloalkyl, cycloalkylalkyl, alkoxyalkyl, cycloalkenyl, alkylamino, amide, thioalkyl, heterocyclyl, heterocyclylalkyl, aryl, heteroaryl, and heteroarylalkyl;
R⁶, R⁷, and R⁸ are each independently selected from the group consisting of H, alkyl, alkoxy, hydroxy, cyano, halogen, alkylamino, cycloalkoxy, amine, halogen, haloalkoxy, haloalkyl, amide, alkoxycarbonyl, and hydroxyalkyl;
R⁹ is selected from the group consisting of H, halogen, and alkyl; and
R¹¹ is selected from the group consisting of H and acyl.

In some embodiments, R² is H and R³ is H.

In some embodiments, R¹ is F, R² is H, and R³ is H.

In some embodiments, R² is F and R³ is H.

In some embodiments, R² is H and R³ is F.

In another embodiment, described herein is a compound represented by Formula I-G:

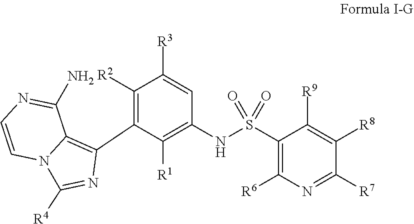

or a pharmaceutically acceptable salt, enantiomer, stereoisomer, or tautomer thereof, wherein:
R¹, and R² are each independently selected from the group consisting of H, alkoxy, alkyl, cyano, haloalkoxy, haloalkyl, and halogen;
R³ is selected from the group consisting of H, alkyl, and halogen;
R⁴ is selected from the group consisting of H, alkyl, alkenyl, alkenylalkyl, alkynyl, alkynylalkyl, cycloalkyl, cycloalkylalkyl, alkoxyalkyl, cycloalkenyl, alkylamino, amide, thioalkyl, heterocyclyl, heterocyclylalkyl, aryl, heteroaryl, and heteroarylalkyl;
R⁶, R⁷, and R⁸ are each independently selected from the group consisting of H, alkyl, alkoxy, hydroxy, cyano, halogen, alkylamino, cycloalkoxy, amine, halogen, haloalkoxy, haloalkyl, amide, alkoxycarbonyl, and hydroxyalkyl; and
R⁹ is selected from the group consisting of H, halogen, and alkyl.

In some embodiments, R² is H and R³ is H.

In some embodiments, R¹ is F, R² is H, and R³ is H.

In some embodiments, R² is F and R³ is H.

In some embodiments, R² is H and R³ is F.

In some embodiments, R⁴ is selected from the group consisting of H, alkyl, (C2-C8)alkenyl, (C2-C8)alkenyl-(C1-C4)alkyl, (C2-C8)alkynyl, (C2-C8)alkynyl-(C1-C4)alkyl, (C3-C8)cycloalkyl, (C3-C8)cycloalkyl-(C1-C4)alkyl, alkoxy-(C1-C4)alkyl, (C3-C8)cycloalkenyl, (C3-C8)cycloalkenyl-(C1-C4)alkyl, alkylamino, amide, thio-(C1-C4)alkyl, heterocyclyl, heterocyclyl-(C1-C4)alkyl, aryl, heteroaryl, and heteroaryl-(C1-C4)alkyl, wherein the alkyl component of the alkylamino is optionally substituted with alkoxy. In some embodiments, R⁴ is selected from the group consisting of H, alkyl, (C3-C8)cycloalkyl, alkylamino, amide, thio-(C1-C4)alkyl, heterocyclyl, and heteroaryl, wherein the alkyl component of the alkylamino is optionally substituted with (C1-C6)alkoxy. In some embodiments, R⁴ is selected from the group consisting of H,

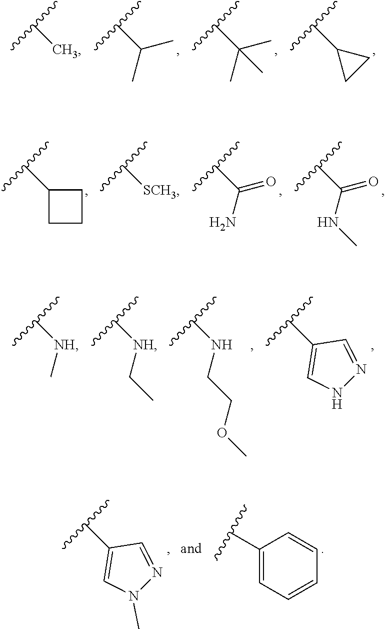

In some embodiments, R⁵ is selected from the group consisting of H, alkyl, (C3-C8)cycloalkyl, alkylamino, hydroxy-(C1-C4)alkyl, hydroxy-(C3-C8)cycloalkyl, alkoxy-(C1-C4)alkyl, alkoxy-(C3-C8)cycloalkyl, halogen, fluoroalkyl, cyano, alkoxy, amine, amino-(C1-C4)alkyl, amino-(C3-C8)cycloalkyl, aminocarbonyl, acylamino, acyloxy-(C1-C4)alkyl, hydroxyimino, alkoxyimino, cyano-(C1-C4)alkyl, heterocyclyl, (C3-C8)cycloalkylamino, (C1-C4)alkoxycarbonyl, and heterocyclyl-(C1-C4)alkyl. In some embodiments, R⁵ is selected from the group consisting of H, alkyl, alkylamino, hydroxy-(C1-C4)alkyl, alkoxy-(C1-C4)alkyl, halogen, fluoroalkyl, cyano, alkoxy, amine, amino-(C1-C4)alkyl, acyloxy-(C1-C4)alkyl, hydroxyimino, alkoxyimino, cyano-(C1-C4)alkyl, heterocyclyl, and alkoxycarbonyl. In some embodiments, R⁵ is selected from the group consisting of H, fluoro, chloro, bromo, CF3,

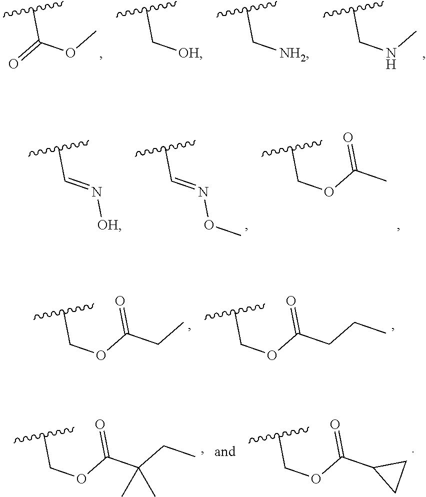

In some embodiments, R⁶, R⁷, and R⁸ are each independently selected from the group consisting of H, alkyl, alkoxy, hydroxy, cyano, halogen, haloalkyl, alkylamino, (C3-C8)cycloalkoxy, amine, halogen, haloalkoxy, haloalkyl, amide, alkoxycarbonyl, and hydroxy-(C1-C4)alkyl. In some embodiments, R⁶, R⁷, and R⁸ are each independently selected from the group consisting of H, alkyl, alkoxy, hydroxy, halogen, and hydroxy-(C1-C4)alkyl.

In some embodiments, R⁶ is selected from the group consisting of H, methyl, methoxy, fluoro, and chloro.

In some embodiments, R⁷ is selected from the group consisting of H, methoxy, fluoro, bromo, and

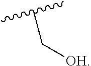 [SMILES: *CO]

In some embodiments, R⁹ is selected from the group consisting of H, halogen, and alkyl. In some embodiments, R⁹ is selected from the group consisting of H and fluoro.

In some embodiments, R¹¹ is selected from the group consisting of H,

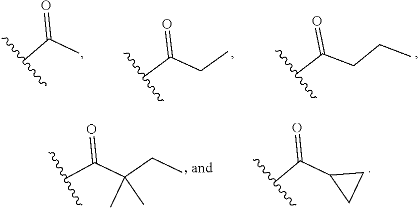

In some embodiments, R¹¹ is selected from the group consisting of H, fluoro, and chloro.

In an embodiment, described herein is a compound selected from the group consisting of:

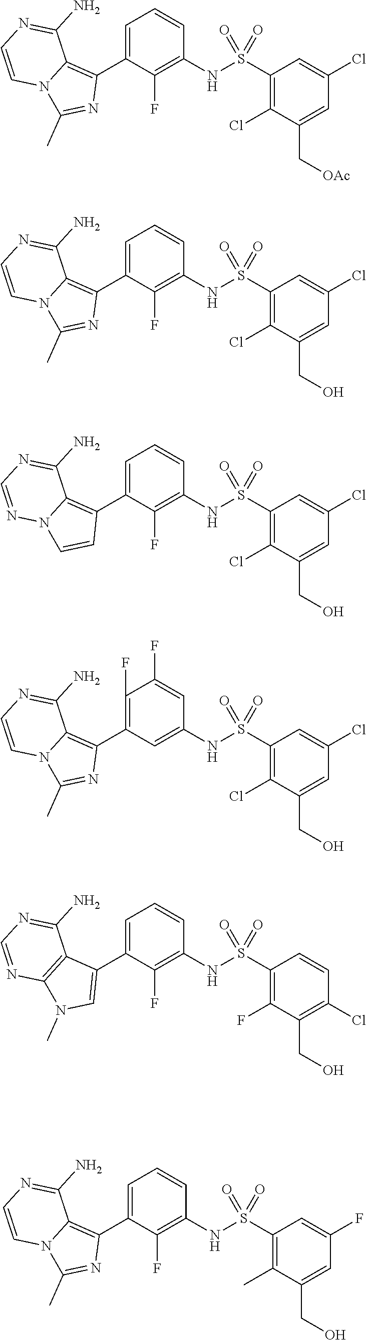 [SMILES: CC(=O)OCc1cc(Cl)cc(S(=O)(=O)Nc2cccc(-c3nc(C)n4ccnc(N)c34)c2F)c1Cl ; Cc1nc(-c2cccc(NS(=O)(=O)c3cc(Cl)cc(CO)c3Cl)c2F)c2c(N)nccn12 ; Nc1ncnn2ccc(-c3cccc(NS(=O)(=O)c4cc(Cl)cc(CO)c4Cl)c3F)c12 ; Cc1nc(-c2cc(NS(=O)(=O)c3cc(Cl)cc(CO)c3Cl)cc(F)c2F)c2c(N)nccn12 ; Cn1cc(-c2cccc(NS(=O)(=O)c3ccc(Cl)c(CO)c3F)c2F)c2c(N)ncnc21 ; Cc1c(CO)cc(F)cc1S(=O)(=O)Nc1cccc(-c2nc(C)n3ccnc(N)c23)c1F]

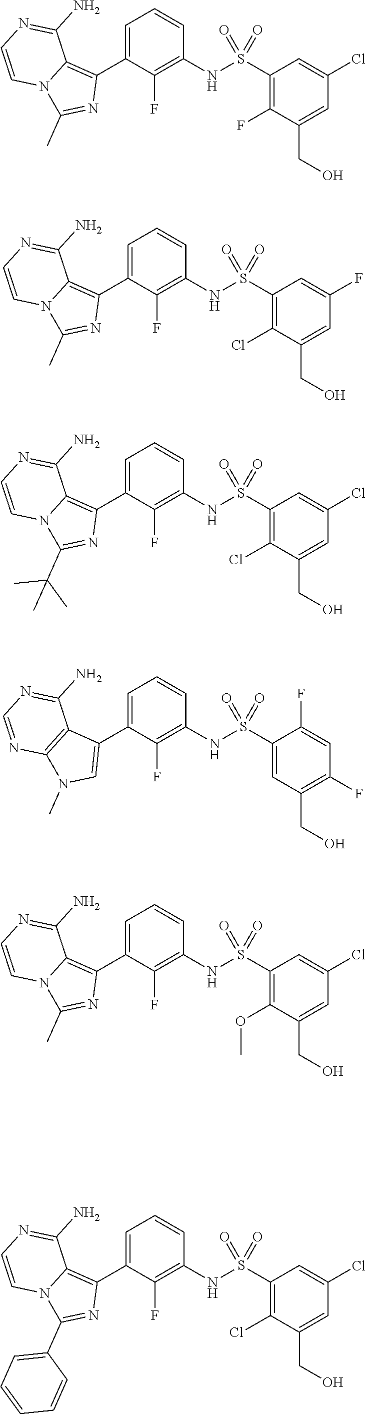 [SMILES: Cc1nc(-c2cccc(NS(=O)(=O)c3cc(Cl)cc(CO)c3F)c2F)c2c(N)nccn12 ; Cc1nc(-c2cccc(NS(=O)(=O)c3cc(F)cc(CO)c3Cl)c2F)c2c(N)nccn12 ; CC(C)(C)c1nc(-c2cccc(NS(=O)(=O)c3cc(Cl)cc(CO)c3Cl)c2F)c2c(N)nccn12 ; Cn1cc(-c2cccc(NS(=O)(=O)c3cc(CO)c(F)cc3F)c2F)c2c(N)ncnc21 ; COc1c(CO)cc(Cl)cc1S(=O)(=O)Nc1cccc(-c2nc(C)n3ccnc(N)c23)c1F ; Nc1nccn2c(-c3ccccc3)nc(-c3cccc(NS(=O)(=O)c4cc(Cl)cc(CO)c4Cl)c3F)c12]

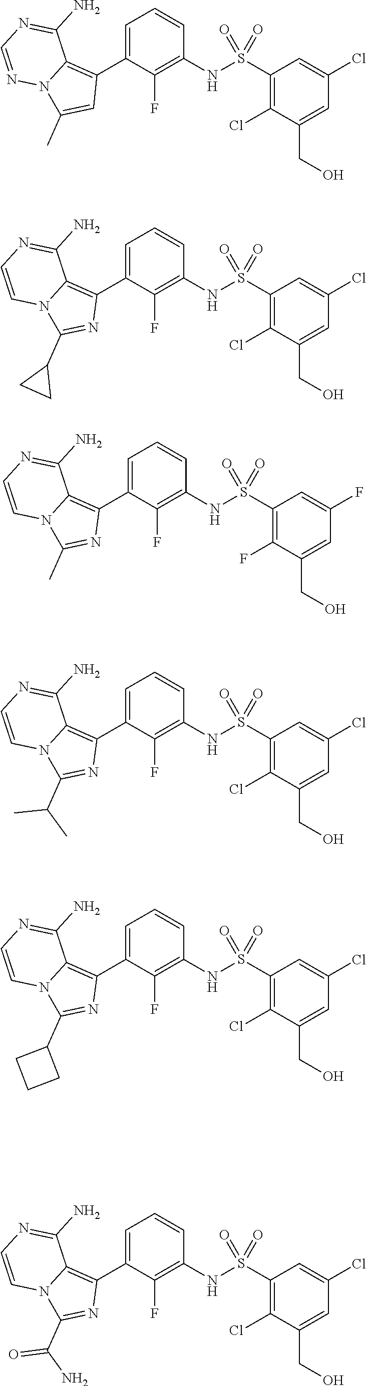 [SMILES: Cc1cc(-c2cccc(NS(=O)(=O)c3cc(Cl)cc(CO)c3Cl)c2F)c2c(N)ncnn12 ; Nc1nccn2c(C3CC3)nc(-c3cccc(NS(=O)(=O)c4cc(Cl)cc(CO)c4Cl)c3F)c12 ; Cc1nc(-c2cccc(NS(=O)(=O)c3cc(F)cc(CO)c3F)c2F)c2c(N)nccn12 ; CC(C)c1nc(-c2cccc(NS(=O)(=O)c3cc(Cl)cc(CO)c3Cl)c2F)c2c(N)nccn12 ; Nc1nccn2c(C3CCC3)nc(-c3cccc(NS(=O)(=O)c4cc(Cl)cc(CO)c4Cl)c3F)c12 ; NC(=O)c1nc(-c2cccc(NS(=O)(=O)c3cc(Cl)cc(CO)c3Cl)c2F)c2c(N)nccn12]

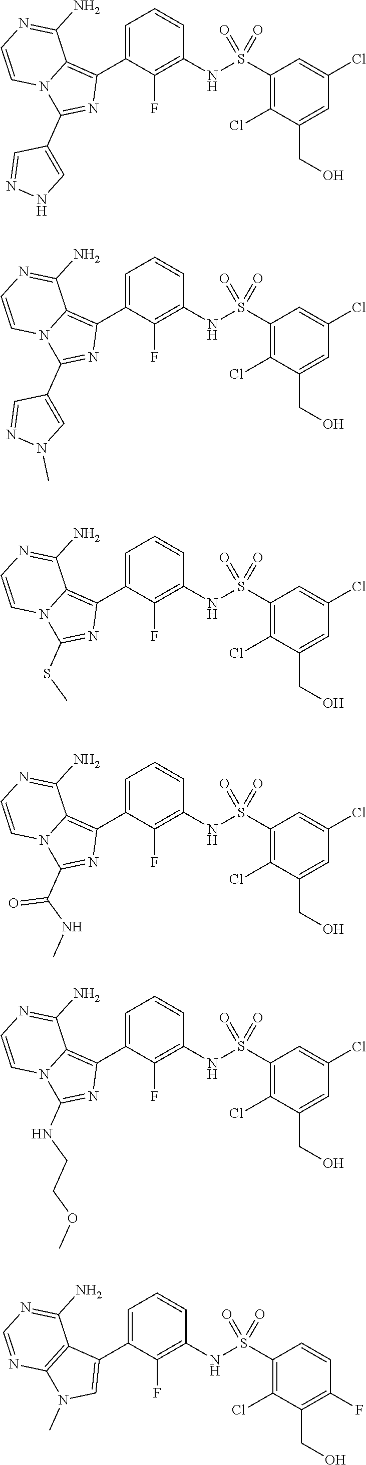

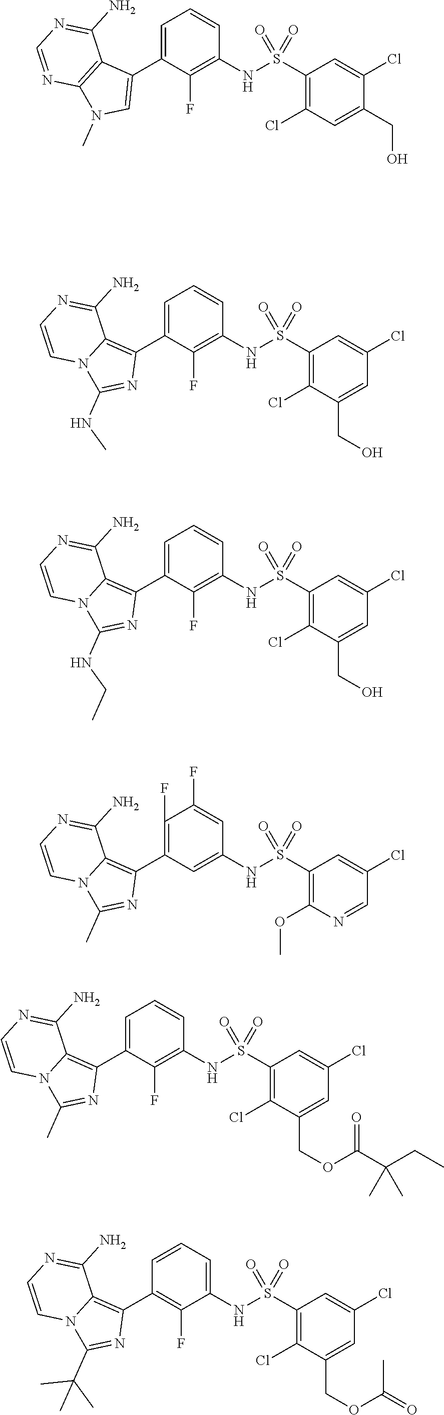 [SMILES: Cn1cc(-c2cccc(NS(=O)(=O)c3cc(Cl)c(CO)cc3Cl)c2F)c2c(N)ncnc21 ; CNc1nc(-c2cccc(NS(=O)(=O)c3cc(Cl)cc(CO)c3Cl)c2F)c2c(N)nccn12 ; CCNc1nc(-c2cccc(NS(=O)(=O)c3cc(Cl)cc(CO)c3Cl)c2F)c2c(N)nccn12 ; COc1ncc(Cl)cc1S(=O)(=O)Nc1cc(F)c(F)c(-c2nc(C)n3ccnc(N)c23)c1 ; CCC(C)(C)C(=O)OCc1cc(Cl)cc(S(=O)(=O)Nc2cccc(-c3nc(C)n4ccnc(N)c34)c2F)c1Cl ; CC(=O)OCc1cc(Cl)cc(S(=O)(=O)Nc2cccc(-c3nc(C(C)(C)C)n4ccnc(N)c34)c2F)c1Cl]

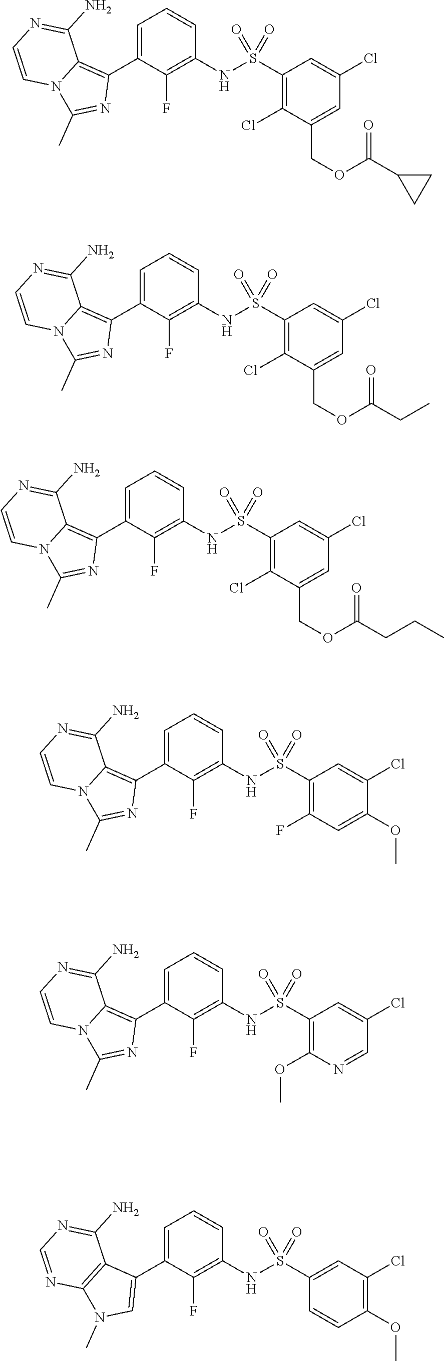 [SMILES: Cc1nc(-c2cccc(NS(=O)(=O)c3cc(Cl)cc(COC(=O)C4CC4)c3Cl)c2F)c2c(N)nccn12 ; CCC(=O)OCc1cc(Cl)cc(S(=O)(=O)Nc2cccc(-c3nc(C)n4ccnc(N)c34)c2F)c1Cl ; CCCC(=O)OCc1cc(Cl)cc(S(=O)(=O)Nc2cccc(-c3nc(C)n4ccnc(N)c34)c2F)c1Cl ; COc1cc(F)c(S(=O)(=O)Nc2cccc(-c3nc(C)n4ccnc(N)c34)c2F)cc1Cl ; COc1ncc(Cl)cc1S(=O)(=O)Nc1cccc(-c2nc(C)n3ccnc(N)c23)c1F ; COc1ccc(S(=O)(=O)Nc2cccc(-c3cn(C)c4ncnc(N)c34)c2F)cc1Cl]

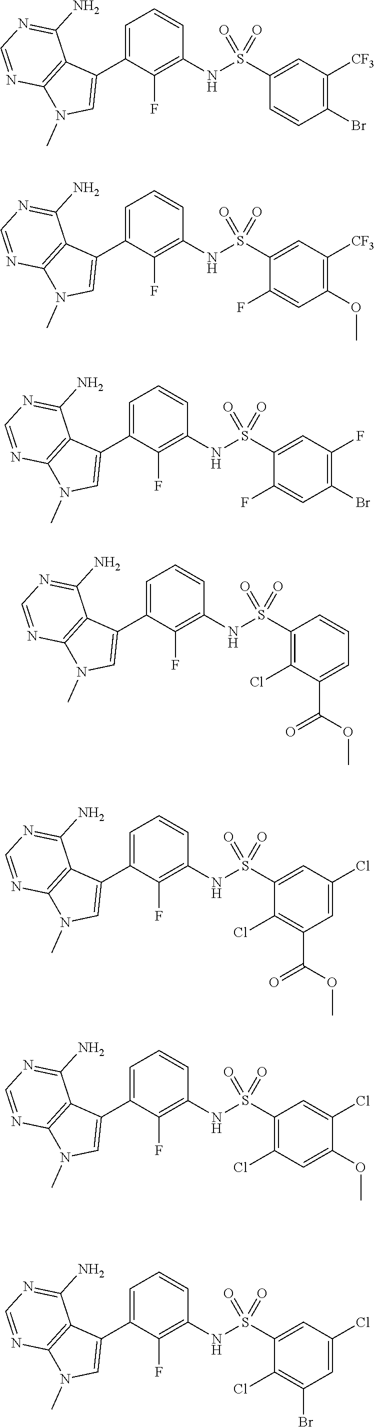

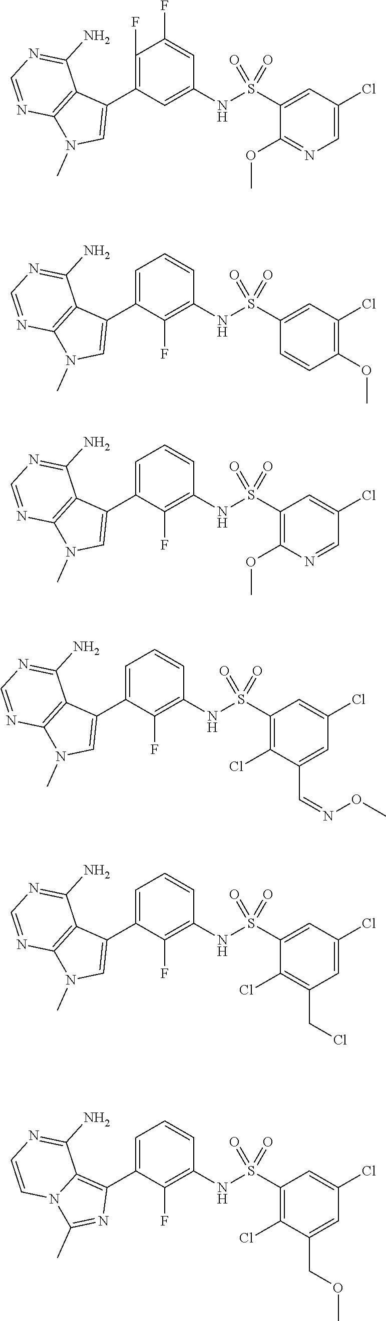 [SMILES: COc1ncc(Cl)cc1S(=O)(=O)Nc1cc(F)c(F)c(-c2cn(C)c3ncnc(N)c23)c1 ; COc1ccc(S(=O)(=O)Nc2cccc(-c3cn(C)c4ncnc(N)c34)c2F)cc1Cl ; COc1ncc(Cl)cc1S(=O)(=O)Nc1cccc(-c2cn(C)c3ncnc(N)c23)c1F ; CO/N=C\c1cc(Cl)cc(S(=O)(=O)Nc2cccc(-c3cn(C)c4ncnc(N)c34)c2F)c1Cl ; Cn1cc(-c2cccc(NS(=O)(=O)c3cc(Cl)cc(CCl)c3Cl)c2F)c2c(N)ncnc21 ; COCc1cc(Cl)cc(S(=O)(=O)Nc2cccc(-c3nc(C)n4ccnc(N)c34)c2F)c1Cl]

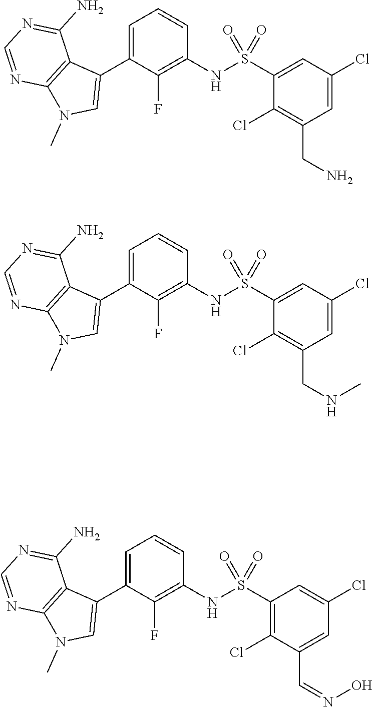 [SMILES: Cn1cc(-c2cccc(NS(=O)(=O)c3cc(Cl)cc(CN)c3Cl)c2F)c2c(N)ncnc21 ; CNCc1cc(Cl)cc(S(=O)(=O)Nc2cccc(-c3cn(C)c4ncnc(N)c34)c2F)c1Cl ; Cn1cc(-c2cccc(NS(=O)(=O)c3cc(Cl)cc(/C=N\O)c3Cl)c2F)c2c(N)ncnc21]

and pharmaceutically acceptable salts, enantiomers, stereoisomers, and tautomers thereof.

### Methods of Treatment

Compounds described herein, e.g., compounds of Formulae I-A, I-B, I-C, I-D, I-E, I-F, and I-G as defined herein, can act as therapeutic agents for diseases driven by GCN2 or PERK kinase, and are useful in the treatment of diseases and disorders in patients in need thereof, such as cancer. Exemplary cancers include, but are not limited to, colorectal cancer, rectal cancer, anal cancer, familial colorectal cancer, hereditary nonpolyposis colorectal cancer, gastrointestinal stromal tumor, lung cancer (e.g., non-small cell lung cancer, small cell lung cancer, malignant mesothelioma), mesothelioma, pancreatic cancer (e.g., pancreatic duct cancer, pancreatic endocrine tumor), pharyngeal cancer, laryngeal cancer, esophagus cancer, gastric cancer (e.g., papillary adenocarcinoma, mucinous adenocarcinoma, adenosquamous carcinoma), duodenal cancer, small intestinal cancer, breast cancer (e.g., invasive ductal carcinoma, ductal carcinoma in situ, inflammatory breast cancer), ovarian cancer (e.g., ovarian epithelial carcinoma, extragonadal germ cell tumor, ovarian germ cell tumor, ovarian low malignant potential tumor), testis tumor, prostate cancer (e.g., hormone-dependent prostate cancer, non-hormone dependent prostate cancer, castration-resistant prostate cancer), liver cancer (e.g., hepatoma, primary liver cancer, extrahepatic bile duct cancer), thyroid cancer (e.g., medullary thyroid carcinoma), renal cancer (e.g., renal cell carcinoma (e.g., clear cell renal cell carcinoma), transitional cell carcinoma of renal pelvis and ureter), uterine cancer (e.g., cervical cancer, uterine body cancer, uterus sarcoma), gestational choriocarcinoma, brain tumor (e.g., medulloblastoma, glioma, pineal astrocytoma, pilocytic astrocytoma, diffuse astrocytoma, anaplastic astrocytoma, hypophyseal adenoma), retinoblastoma, skin cancer (e.g., basal cell carcinoma, malignant melanoma, melanoma), sarcoma (e.g., rhabdomyosarcoma, leiomyosarcoma, soft tissue sarcoma, spindle cell sarcoma, osteosarcoma), fibrosarcoma, malignant bone tumor, urinary bladder cancer, hematologic cancer (e.g., multiple myeloma, leukemia, acute myeloid leukemia (AMIL), acute lymphoblastic leukemia (ALL), including blast crisis of chronic leukemia), malignant lymphoma, Hodgkin's disease, chronic myeloproliferative disease), cancer of unknown primary nucleus], cancer growth inhibitor, cancer metastasis inhibitor, apoptosis promoter, and for the prophylaxis or treatment of precancerous lesion (e.g., bone marrow myelodysplastic syndrome).

Also described herein, in one embodiment, is a method of treating a disease caused by a dysregulation of an integrated stress response and/or the unfolded protein response in a patient in need thereof, comprising administering to the patient a therapeutically effective amount of a compound described herein (e.g., a compound of Formulae I-A, I-B, I-C, I-D, I-E, I-F, and I-G described herein), or a pharmaceutically acceptable salt thereof, or of a pharmaceutical composition described herein. In some embodiments, the dysregulation of the integrated stress response and/or the unfolded protein response is caused by GCN2 kinase. In some embodiments, the dysregulation of the integrated stress response and/or the unfolded protein response is caused by PERK kinase. In some embodiments, the dysregulation of the integrated stress response is caused by GCN2 kinase. In some embodiments, the dysregulation of the unfolded protein response is caused by PERK kinase. In some embodiments, the dysregulation of the integrated stress response is caused by activation of a kinase selected from the group consisting of PERK kinase and GCN2 kinase. In some embodiments, the dysregulation of the integrated stress response is caused by activation of GCN2 kinase. In some embodiments, the dysregulation of the integrated stress response is caused by activation of PERK kinase.

Also described herein, in one embodiment, is a method of treating a disease caused by a dysregulation of an integrated stress response and/or an unfolded protein response in a patient in need thereof, comprising administering to the patient a therapeutically effective amount of a compound described herein (e.g., a compound of Formulae I-A, I-B, I-C, I-D, I-E, I-F, and I-G described herein), or a pharmaceutically acceptable salt thereof, or of a pharmaceutical composition described herein. In some embodiments, the dysregulation of the integrated stress response and/or the unfolded protein response is caused by activation of a kinase selected from the group consisting of PERK kinase and GCN2 kinase. In some embodiments, the dysregulation of the integrated stress response is caused by activation of GCN2 kinase. In some embodiments, the dysregulation of the unfolded protein response is caused by activation of PERK kinase.

In another embodiment, described herein is a method of modulating the activity of GCN2 kinase in a patient in need thereof, comprising administering to the patient a therapeutically effective amount of a compound described herein, or a pharmaceutically acceptable salt thereof, or of a pharmaceutical composition described herein.

In another embodiment, described herein is a method of activating GCN2 kinase in a patient in need thereof, comprising administering to the patient a therapeutically effective amount of a compound described herein, or a pharmaceutically acceptable salt thereof, or of a pharmaceutical composition described herein.

In another embodiment, described herein is a method of modulating the activity of PERK kinase in a patient in need thereof, comprising administering to the patient a therapeutically effective amount of a compound described herein, or a pharmaceutically acceptable salt thereof, or of a pharmaceutical composition described herein.

In another embodiment, described herein is a method of activating PERK kinase in a patient in need thereof, comprising administering to the patient a therapeutically effective amount of a compound described herein, or a pharmaceutically acceptable salt thereof, or of a pharmaceutical composition described herein.

In another embodiment, described herein is a method of inhibiting GCN2 kinase and inhibiting PERK kinase in a patient in need thereof, comprising administering to the patient a therapeutically effective amount of a compound described herein, or a pharmaceutically acceptable salt thereof, or of a pharmaceutical composition described herein.

In another embodiment, described herein is a method of inhibiting the activity of GCN2 kinase in a patient in need thereof, comprising administering to the patient a therapeutically effective amount of a compound described herein, or a pharmaceutically acceptable salt thereof, or of a pharmaceutical composition described herein.

In another embodiment, described herein is a method of inhibiting the activity of PERK kinase in a patient in need thereof, comprising administering to the patient a therapeutically effective amount of a compound described herein, or a pharmaceutically acceptable salt thereof, or of a pharmaceutical composition described herein.

In another embodiment, described herein is a method of treating a cancer in a patient in need thereof, comprising administering to the patient a therapeutically effective amount of a compound described herein (e.g., a compound of Formulae I-A, I-B, I-C, I-D, I-E, I-F, and I-G described herein), or a pharmaceutically acceptable salt thereof, or of a pharmaceutical composition described herein. In some embodiments, the cancer is selected from the group consisting of colorectal cancer, lung cancer, mesothelioma, pancreatic cancer, pharyngeal cancer, laryngeal cancer, esophagus cancer, gastric, duodenal cancer, small intestinal cancer, breast cancer, ovarian cancer, testis tumor, prostate, liver cancer, thyroid cancer, renal cancer, uterine cancer, gestational choriocarcinoma, brain tumor, retinoblastoma, skin cancer, melanoma, sarcoma, fibrosarcoma, malignant bone tumor, urinary bladder cancer, hematologic cancer, leukemia, acute myeloid leukemia, acute lymphoblastic leukemia, and malignant lymphoma. In some embodiments, the cancer is selected from the group consisting of colorectal cancer, lung cancer, mesothelioma, pancreatic cancer, pharyngeal cancer, laryngeal cancer, esophagus cancer, gastric, duodenal cancer, small intestinal cancer, breast cancer, ovarian cancer, testis tumor, prostate, liver cancer, thyroid cancer, renal cancer, uterine cancer, gestational choriocarcinoma, brain tumor, retinoblastoma, skin cancer, melanoma, sarcoma, fibrosarcoma, malignant bone tumor, urinary bladder cancer, hematologic cancer, leukemia, acute myeloid leukemia, acute lymphoblastic leukemia, chronic lymphocytic leukemia, chronic myeloid leukemia, multiple myeloma, B-cell lymphoma, Follicular lymphoma, mantle cell lymphoma, diffuse large B cell lymphoma, T-cell lymphoma, erythroleukemia, histocyctic lymphoma, Waldenstrom macroglobulinemia, and malignant lymphoma. In some embodiments, the cancer is leukemia. In some embodiments, the cancer is acute myeloid leukemia. In some embodiments, the cancer is acute lymphoblastic leukemia. In some embodiments, the cancer is fibrosarcoma. In some embodiments, the cancer is multiple myeloma. In some embodiments, the cancer is lymphoma. In some embodiments, the cancer is B-cell lymphoma. In some embodiments, the cancer is T cell lymphoma.

In an embodiment, described herein is a method of treating amyloidosis in a patient in need thereof, comprising administering to the patient a compound described herein, or pharmaceutically acceptable salt thereof, or pharmaceutical composition described herein. In an embodiment, described herein is a method of treating light chain amyloidosis in a patient in need thereof, comprising administering to the patient a compound described herein, or pharmaceutically acceptable salt thereof, or pharmaceutical composition described herein.

In some embodiments, the methods described herein further comprises administering to the patient a therapeutically effective amount of one or more therapeutic agents. In some embodiments, the one or more therapeutic agents is selected from the group consisting of L-asparaginase, pegylated asparaginase, a PERK inhibitor, a mTOR inhibitor, an immunomodulatory agent, a MAPK pathway inhibitor, a MEK inhibitor, an ERK inhibitor, and a Ras inhibitor. In some embodiments, the one or more therapeutic agents is selected from the group consisting of an IMiD agent, a proteasome inhibitor, a steroid, an anti-CD38 agent, an anti-CD20 agent, a Bcl-2 inhibitor, a PI3K inhibitor, a Bi-specific antibody, a nucleoside analog, a BTK inhibitor, a DNA alkylating agent, an EZH2 inhibitor, an anthracycline, a topoisomerase inhibitor, a platin, a tyrosine kinase inhibitor, an HDAC inhibitor, a nuclear export inhibitor, an anti-microtubule agent L-asparaginase, pegylated asparaginase, a PERK inhibitor, a mTOR inhibitor, an immunomodulatory agent, a MAPK pathway inhibitor, a MEK inhibitor, an ERK inhibitor, and a Ras inhibitor. In some embodiments, the one or more therapeutic agents is selected from the group consisting of L-asparaginase, pegaspargase, calaspargase pegol—mnkl, bortezomib, carfilzomib, ixazomib, thalidomide, pomalidomide, lenalidomide, dexamethasone, prednisone, daratumumab, daratumumab/hyaluronidase, isatuximab, rituximab, obinutuzumab, venetoclax, idelalisib, copanlisib, duvelisib, umbralisib, gemcitabine, cytarabine, ibrutinib, acalabrutinib, zanubrutinib, bendamustine, cyclophosphamide, tazemetostat, doxorubicin, daunorubicin, etoposide, oxaloplatin, carboplatin, cisplatinbosutinib, dasatinib, imatinib, nilotinib, ponatinib, panobinostat, selinexor, vincristine, JZP-458, eryaspase, PF745 (JZP-341), asparaginase Erwinia chrysanthemi (crisantaspase), Escherichia coli asparaginase (colaspase), an anti-PD1 agent, an anti-PDL1 agent, and an anti-CTLA4 agent.

In another embodiment, described herein is a method of treating a disease selected from a GCN2 associated disease and a PERK associated disease, in a patient in need thereof, comprising administering to the patient a therapeutically effective amount of a compound described herein (e.g., a compound of Formulae I-A, I-B, I-C, I-D, I-E, I-F, and I-G described herein), or a pharmaceutically acceptable salt thereof, or of a pharmaceutical composition described herein. In some embodiments, the disease is a GCN2 associated disease. In some embodiments, the disease is a PERK associated disease. In some embodiments, the disease is a cancer. In some embodiments, the cancer is selected from the group consisting of colorectal cancer, lung cancer, mesothelioma, pancreatic cancer, pharyngeal cancer, laryngeal cancer, esophagus cancer, gastric, duodenal cancer, small intestinal cancer, breast cancer, ovarian cancer, testis tumor, prostate, liver cancer, thyroid cancer, renal cancer, uterine cancer, gestational choriocarcinoma, brain tumor, retinoblastoma, skin cancer, melanoma, sarcoma, fibrosarcoma, malignant bone tumor, urinary bladder cancer, hematologic cancer, leukemia, acute myeloid leukemia, acute lymphoblastic leukemia, and malignant lymphoma. In some embodiments, the cancer is selected from the group consisting of colorectal cancer, lung cancer, mesothelioma, pancreatic cancer, pharyngeal cancer, laryngeal cancer, esophagus cancer, gastric, duodenal cancer, small intestinal cancer, breast cancer, ovarian cancer, testis tumor, prostate, liver cancer, thyroid cancer, renal cancer, uterine cancer, gestational choriocarcinoma, brain tumor, retinoblastoma, skin cancer, melanoma, sarcoma, fibrosarcoma, malignant bone tumor, urinary bladder cancer, hematologic cancer, leukemia, acute myeloid leukemia, acute lymphoblastic leukemia, chronic lymphocytic leukemia, chronic myeloid leukemia, multiple myeloma, B-cell lymphoma, Follicular lymphoma, mantle cell lymphoma, diffuse large B cell lymphoma, T-cell lymphoma, erythroleukemia, histocyctic lymphoma, Waldenstrom macroglobulinemia, and malignant lymphoma. In some embodiments, the cancer is leukemia. In some embodiments, the cancer is acute myeloid leukemia. In some embodiments, the cancer is acute lymphoblastic leukemia. In some embodiments, the cancer is fibrosarcoma. In some embodiments, the cancer is multiple myeloma. In some embodiments, the cancer is lymphoma. In some embodiments, the cancer is B-cell lymphoma. In some embodiments, the cancer is T cell lymphoma. In some embodiments, the disease is amyloidosis. In some embodiments, the disease is light chain amyloidosis.

In another embodiment, described herein is a method of treating a disease selected from a GCN2 associated disease and a PERK associated disease, in a patient in need thereof, comprising administering to the patient a therapeutically effective amount of a compound described herein (e.g., a compound of Formulae I-A, I-B, I-C, I-D, I-E, I-F, and I-G described herein), or a pharmaceutically acceptable salt thereof, and a therapeutically effective amount of one or more therapeutic agents. In some embodiments, the disease is a GCN2 associated disease. In some embodiments, the disease is a PERK associated disease. In some embodiments, the disease is a cancer. In some embodiments, the cancer is selected from the group consisting of colorectal cancer, lung cancer, mesothelioma, pancreatic cancer, pharyngeal cancer, laryngeal cancer, esophagus cancer, gastric, duodenal cancer, small intestinal cancer, breast cancer, ovarian cancer, testis tumor, prostate, liver cancer, thyroid cancer, renal cancer, uterine cancer, gestational choriocarcinoma, brain tumor, retinoblastoma, skin cancer, melanoma, sarcoma, fibrosarcoma, malignant bone tumor, urinary bladder cancer, hematologic cancer, leukemia, acute myeloid leukemia, acute lymphoblastic leukemia, and malignant lymphoma. In some embodiments, the cancer is selected from the group consisting of colorectal cancer, lung cancer, mesothelioma, pancreatic cancer, pharyngeal cancer, laryngeal cancer, esophagus cancer, gastric, duodenal cancer, small intestinal cancer, breast cancer, ovarian cancer, testis tumor, prostate, liver cancer, thyroid cancer, renal cancer, uterine cancer, gestational choriocarcinoma, brain tumor, retinoblastoma, skin cancer, melanoma, sarcoma, fibrosarcoma, malignant bone tumor, urinary bladder cancer, hematologic cancer, leukemia, acute myeloid leukemia, acute lymphoblastic leukemia, chronic lymphocytic leukemia, chronic myeloid leukemia, multiple myeloma, B-cell lymphoma, Follicular lymphoma, mantle cell lymphoma, diffuse large B cell lymphoma, T-cell lymphoma, erythroleukemia, histocyctic lymphoma, Waldenstrom macroglobulinemia, and malignant lymphoma. In some embodiments, the cancer is leukemia. In some embodiments, leukemia is acute myeloid leukemia. In some embodiments, leukemia is acute lymphoblastic leukemia. In some embodiments, the cancer is fibrosarcoma. In some embodiments, the cancer is multiple myeloma. In some embodiments, the cancer is lymphoma. In some embodiments, the cancer is B-cell lymphoma. In some embodiments, the cancer is T cell lymphoma. In some embodiments, the disease is amyloidosis. In some embodiments, the disease is light chain amyloidosis. In some embodiments, the one or more therapeutic agents is selected from the group consisting of L-asparaginase, pegylated asparaginase, a PERK inhibitor, a mTOR inhibitor, an immunomodulatory agent, a MAPK pathway inhibitor, a MEK inhibitor, an ERK inhibitor, and a Ras inhibitor. In some embodiments, the one or more therapeutic agents is selected from the group consisting of an IMiD agent, a proteasome inhibitor, a steroid, an anti-CD38 agent, an anti-CD20 agent, a Bcl-2 inhibitor, a PI3K inhibitor, a Bi-specific antibody, a nucleoside analog, a BTK inhibitor, a DNA alkylating agent, an EZH2 inhibitor, an anthracycline, a topoisomerase inhibitor, a platin, a tyrosine kinase inhibitor, an HDAC inhibitor, a nuclear export inhibitor, an anti-microtubule agent L-asparaginase, pegylated asparaginase, a PERK inhibitor, a mTOR inhibitor, an immunomodulatory agent, a MAPK pathway inhibitor, a MEK inhibitor, an ERK inhibitor, and a Ras inhibitor. In some embodiments, the one or more therapeutic agents is selected from the group consisting of L-asparaginase, pegaspargase, calaspargase pegol—mnkl, bortezomib, carfilzomib, ixazomib, thalidomide, pomalidomide, lenalidomide, dexamethasone, prednisone, daratumumab, daratumumab/hyaluronidase, isatuximab, rituximab, obinutuzumab, venetoclax, idelalisib, copanlisib, duvelisib, umbralisib, gemcitabine, cytarabine, ibrutinib, acalabrutinib, zanubrutinib, bendamustine, cyclophosphamide, tazemetostat, doxorubicin, daunorubicin, etoposide, oxaloplatin, carboplatin, cisplatinbosutinib, dasatinib, imatinib, nilotinib, ponatinib, panobinostat, selinexor, vincristine, JZP-458, eryaspase, PF745 (JZP-341), asparaginase Erwinia chrysanthemi (crisantaspase), Escherichia coli asparaginase (colaspase), an anti-PD1 agent, an anti-PDL1 agent, and an anti-CTLA4 agent.

In an embodiment, described herein is a compound described herein, or a pharmaceutically acceptable salt thereof, or the pharmaceutical composition described herein, for use in therapy.

In an embodiment, described herein is a compound described herein, or a pharmaceutically acceptable salt thereof, or the pharmaceutical composition described herein, for use in treating a disease caused by a dysregulation of an integrated stress response and/or the unfolded protein response in a patient in need thereof. In some embodiments, the dysregulation of the integrated stress response and/or the unfolded protein response is caused by a kinase selected from the group consisting of PERK kinase and GCN2 kinase. In some embodiments, the dysregulation of the integrated stress response and/or the unfolded protein response is caused by GCN2 kinase. In some embodiments, the dysregulation of the integrated stress response and/or the unfolded protein response is caused by PERK kinase. In some embodiments, the dysregulation of the integrated stress response is caused by GCN2 kinase. In some embodiments, the dysregulation of the unfolded protein response is caused by PERK kinase. In some embodiments, the dysregulation of the integrated stress response is caused by activation of a kinase selected from the group consisting of PERK kinase and GCN2 kinase. In some embodiments, the dysregulation of the integrated stress response is caused by activation of GCN2 kinase. In some embodiments, the dysregulation of the integrated stress response is caused by activation of PERK kinase.

In an embodiment, described herein is a compound described herein, or a pharmaceutically acceptable salt thereof, or the pharmaceutical composition described herein, for use in modulating the activity of GCN2 kinase in a patient in need thereof.

In an embodiment, described herein is a compound described herein, or a pharmaceutically acceptable salt thereof, or the pharmaceutical composition described herein, for use in activating GCN2 kinase in a patient in need thereof.

In an embodiment, described herein is a compound described herein, or a pharmaceutically acceptable salt thereof, or the pharmaceutical composition described herein, for use in modulating the activity of PERK kinase in a patient in need thereof.

In an embodiment, described herein is a compound described herein, or a pharmaceutically acceptable salt thereof, or the pharmaceutical composition described herein, for use in activating PERK kinase in a patient in need thereof.

In an embodiment, described herein is a compound described herein, or a pharmaceutically acceptable salt thereof, or the pharmaceutical composition described herein, for use in inhibiting GCN2 kinase and inhibiting PERK kinase in a patient in need thereof.

In an embodiment, described herein is a compound described herein, or a pharmaceutically acceptable salt thereof, or the pharmaceutical composition described herein, for use in inhibiting the activity of GCN2 kinase in a patient in need thereof.

In another embodiment, described herein is a compound described herein, or a pharmaceutically acceptable salt thereof, or the pharmaceutical composition described herein, for use in inhibiting the activity of PERK kinase in a patient in need thereof.

In an embodiment, described herein is a compound described herein, or a pharmaceutically acceptable salt thereof, or the pharmaceutical composition described herein, for use in treating a cancer in a patient in need thereof. In some embodiments, the cancer is selected from the group consisting of colorectal cancer, lung cancer, mesothelioma, pancreatic cancer, pharyngeal cancer, laryngeal cancer, esophagus cancer, gastric, duodenal cancer, small intestinal cancer, breast cancer, ovarian cancer, testis tumor, prostate, liver cancer, thyroid cancer, renal cancer, uterine cancer, gestational choriocarcinoma, brain tumor, retinoblastoma, skin cancer, melanoma, sarcoma, fibrosarcoma, malignant bone tumor, urinary bladder cancer, hematologic cancer, leukemia, acute myeloid leukemia, acute lymphoblastic leukemia, chronic lymphocytic leukemia, chronic myeloid leukemia, multiple myeloma, B-cell lymphoma, follicular lymphoma, mantle cell lymphoma, diffuse large B cell lymphoma, T-cell lymphoma, erythroleukemia, histocyctic lymphoma, Waldenstrom macroglobulinemia, and malignant lymphoma. In some embodiments, the cancer is leukemia. In some embodiments, the cancer is acute myeloid leukemia. In some embodiments, the cancer is acute lymphoblastic leukemia. In some embodiments, the cancer is fibrosarcoma. In some embodiments, the cancer is multiple myeloma. In some embodiments, the cancer is lymphoma. In some embodiments, the cancer is B-cell lymphoma. In some embodiments, the cancer is T cell lymphoma.

In an embodiment, described herein is a compound described herein, or a pharmaceutically acceptable salt thereof, or the pharmaceutical composition described herein, for use in treating amyloidosis in a patient in need thereof. In an embodiment, described herein is a compound described herein, or a pharmaceutically acceptable salt thereof, or the pharmaceutical composition described herein, for use in treating light chain amyloidosis in a patient in need thereof.

In some embodiments, the compound or composition for use described herein further comprises use of one or more therapeutic agents. In some embodiments, the one or more therapeutic agents is selected from the group consisting of L-asparaginase, pegylated asparaginase, a PERK inhibitor, a mTOR inhibitor, an immunomodulatory agent, a MAPK pathway inhibitor, a MEK inhibitor, an ERK inhibitor, and a Ras inhibitor. In some embodiments, the one or more therapeutic agents is selected from the group consisting of an IMiD agent, a proteasome inhibitor, a steroid, an anti-CD38 agent, an anti-CD20 agent, a Bcl-2 inhibitor, a PI3K inhibitor, a Bi-specific antibody, a nucleoside analog, a BTK inhibitor, a DNA alkylating agent, an EZH2 inhibitor, an anthracycline, a topoisomerase inhibitor, a platin, a tyrosine kinase inhibitor, an HDAC inhibitor, a nuclear export inhibitor, an anti-microtubule agent L-asparaginase, pegylated asparaginase, a PERK inhibitor, a mTOR inhibitor, an immunomodulatory agent, a MAPK pathway inhibitor, a MEK inhibitor, an ERK inhibitor, and a Ras inhibitor. In some embodiments, the one or more therapeutic agents is selected from the group consisting of L-asparaginase, pegaspargase, calaspargase pegol—mnkl, bortezomib, carfilzomib, ixazomib, thalidomide, pomalidomide, lenalidomide, dexamethasone, prednisone, daratumumab, daratumumab/hyaluronidase, isatuximab, rituximab, obinutuzumab, venetoclax, idelalisib, copanlisib, duvelisib, umbralisib, gemcitabine, cytarabine, ibrutinib, acalabrutinib, zanubrutinib, bendamustine, cyclophosphamide, tazemetostat, doxorubicin, daunorubicin, etoposide, oxaloplatin, carboplatin, cisplatinbosutinib, dasatinib, imatinib, nilotinib, ponatinib, panobinostat, selinexor, vincristine, JZP-458, eryaspase, PF745 (JZP-341), asparaginase Erwinia chrysanthemi (crisantaspase), Escherichia coli asparaginase (colaspase), an anti-PD1 agent, an anti-PDL1 agent, and an anti-CTLA4 agent.

In an embodiment, described herein is a compound described herein, or a pharmaceutically acceptable salt thereof, or the pharmaceutical composition described herein, for use in treating a disease selected from a GCN2 associated disease and a PERK associated disease, in a patient in need thereof. In some embodiments, the disease is a GCN2 associated disease. In some embodiments, the disease is a PERK associated disease. In some embodiments, the disease is a cancer. In some embodiments, the cancer is selected from the group consisting of colorectal cancer, lung cancer, mesothelioma, pancreatic cancer, pharyngeal cancer, laryngeal cancer, esophagus cancer, gastric, duodenal cancer, small intestinal cancer, breast cancer, ovarian cancer, testis tumor, prostate, liver cancer, thyroid cancer, renal cancer, uterine cancer, gestational choriocarcinoma, brain tumor, retinoblastoma, skin cancer, melanoma, sarcoma, fibrosarcoma, malignant bone tumor, urinary bladder cancer, hematologic cancer, leukemia, acute myeloid leukemia, acute lymphoblastic leukemia, chronic lymphocytic leukemia, chronic myeloid leukemia, multiple myeloma, B-cell lymphoma, follicular lymphoma, mantle cell lymphoma, diffuse large B cell lymphoma, T-cell lymphoma, erythroleukemia, histocyctic lymphoma, Waldenstrom macroglobulinemia, and malignant lymphoma. In some embodiments, the cancer is leukemia. In some embodiments, the cancer is acute myeloid leukemia. In some embodiments, the cancer is acute lymphoblastic leukemia. In some embodiments, the cancer is fibrosarcoma. In some embodiments, the cancer is multiple myeloma. In some embodiments, the cancer is lymphoma. In some embodiments, the cancer is B-cell lymphoma. In some embodiments, the cancer is T cell lymphoma. In some embodiments, the disease is amyloidosis. In some embodiments, the disease is light chain amyloidosis.

In an embodiment, described herein is a compound described herein, or a pharmaceutically acceptable salt thereof, or the pharmaceutical composition described herein, for use in treating a disease selected from a GCN2 associated disease and a PERK associated disease, in a patient in need thereof. In some embodiments, the disease is a GCN2 associated disease. In some embodiments, the disease is a PERK associated disease. In some embodiments, the disease is a cancer. In some embodiments, the cancer is selected from the group consisting of colorectal cancer, lung cancer, mesothelioma, pancreatic cancer, pharyngeal cancer, laryngeal cancer, esophagus cancer, gastric, duodenal cancer, small intestinal cancer, breast cancer, ovarian cancer, testis tumor, prostate, liver cancer, thyroid cancer, renal cancer, uterine cancer, gestational choriocarcinoma, brain tumor, retinoblastoma, skin cancer, melanoma, sarcoma, fibrosarcoma, malignant bone tumor, urinary bladder cancer, hematologic cancer, leukemia, acute myeloid leukemia, acute lymphoblastic leukemia, chronic lymphocytic leukemia, chronic myeloid leukemia, multiple myeloma, B-cell lymphoma, follicular lymphoma, mantle cell lymphoma, diffuse large B cell lymphoma, T-cell lymphoma, erythroleukemia, histocyctic lymphoma, Waldenstrom macroglobulinemia, and malignant lymphoma. In some embodiments, the cancer is leukemia. In some embodiments, leukemia is acute myeloid leukemia. In some embodiments, leukemia is acute lymphoblastic leukemia. In some embodiments, the cancer is fibrosarcoma. In some embodiments, the cancer is multiple myeloma. In some embodiments, the cancer is lymphoma. In some embodiments, the cancer is B-cell lymphoma. In some embodiments, the cancer is T cell lymphoma. In some embodiments, the disease is amyloidosis. In some embodiments, the disease is light chain amyloidosis. In some embodiments, the one or more therapeutic agents is selected from the group consisting of an IMiD agent, a proteasome inhibitor, a steroid, an anti-CD38 agent, an anti-CD20 agent, a Bcl-2 inhibitor, a PI3K inhibitor, a Bi-specific antibody, a nucleoside analog, a BTK inhibitor, a DNA alkylating agent, an EZH2 inhibitor, an anthracycline, a topoisomerase inhibitor, a platin, a tyrosine kinase inhibitor, an HDAC inhibitor, a nuclear export inhibitor, an anti-microtubule agent, L-asparaginase, pegylated asparaginase, a PERK inhibitor, a mTOR inhibitor, an immunomodulatory agent, a MAPK pathway inhibitor, a MEK inhibitor, an ERK inhibitor, and a Ras inhibitor. In some embodiments, the one or more therapeutic agents is selected from the group consisting of bortezomib, carfilzomib, ixazomib, thalidomide, pomalidomide, lenalidomide, dexamethasone, prednisone, daratumumab, daratumumab/hyaluronidase, isatuximab, rituximab, obinutuzumab, venetoclax, idelalisib, copanlisib, duvelisib, umbralisib, gemcitabine, cytarabine, ibrutinib, acalabrutinib, zanubrutinib, bendamustine, cyclophosphamide, tazemetostat, doxorubicin, daunorubicin, etoposide, oxaloplatin, carboplatin, cisplatinbosutinib, dasatinib, imatinib, nilotinib, ponatinib, panobinostat, selinexor, vincristine, L-asparaginase, pegaspargase, calaspargase pegol-mnkl, JZP-458, eryaspase, PF745 (JZP-341), asparaginase Erwinia chrysanthemi (crisantaspase), Escherichia coli asparaginase (colaspase), anti-PD1, anti-PDL1, and anti-CTLA4.

The compounds provided herein may be administered to patients (animals and humans) in need of such treatment in dosages that will provide optimal pharmaceutical efficacy. It will be appreciated that the dose required for use in any particular application will vary from patient to patient, not only with the particular compound or composition selected, but also with the route of administration, the nature of the condition being treated, the age and condition of the patient, concurrent medication or special diets then being followed by the patient, and other factors which those skilled in the art will recognize, with the appropriate dosage ultimately being at the discretion of the attendant physician. For treating clinical conditions and diseases noted above, a compound provided herein may be administered orally, subcutaneously, topically, parenterally, by inhalation spray or rectally in dosage unit formulations containing conventional non-toxic pharmaceutically acceptable carriers, adjuvants and vehicles. Parenteral administration may include subcutaneous injections, intravenous or intramuscular injections or infusion techniques.

Treatment can be continued for as long or as short a period as desired. The compositions may be administered on a regimen of, for example, one to four or more times per day. A suitable treatment period can be, for example, at least about one week, at least about two weeks, at least about one month, at least about six months, at least about 1 year, or indefinitely. A treatment period can terminate when a desired result is achieved.

### Combination Therapy

Compounds described herein, e.g., a compound of Formulae I-A, I-B, I-C, I-D, I-E, I-F, and I-G as defined herein, can be administered in combination with one or more additional therapeutic agents to treat a disorder described herein, such as a cancer described herein. For example, provided in the present disclosure is a pharmaceutical composition comprising a compound described herein, e.g., a compound of Formulae I-A, I-B, I-C, I-D, I-E, I-F, and I-G as defined herein, one or more additional therapeutic agents, and a pharmaceutically acceptable excipient. In some embodiments, a compound of Formulae I-A, I-B, I-C, I-D, I-E, I-F, and I-G as defined herein, and one additional therapeutic agent is administered. In some embodiments, a compound of Formulae I-A, I-B, I-C, I-D, I-E, I-F, and I-G as defined herein, and two additional therapeutic agents are administered. In some embodiments, a compound of Formulae I-A, I-B, I-C, I-D, I-E, I-F, and I-G as defined herein, and three additional therapeutic agents are administered. Combination therapy can be achieved by administering two or more therapeutic agents, each of which is formulated and administered separately. For example, a compound of Formulae I-A, I-B, I-C, I-D, I-E, I-F, and I-G as defined herein, and an additional therapeutic agent can be formulated and administered separately. Combination therapy can also be achieved by administering two or more therapeutic agents in a single formulation, for example a pharmaceutical composition comprising a compound of Formulae I-A, I-B, I-C, I-D, I-E, I-F, and I-G as one therapeutic agent and one or more additional therapeutic agents such as a chemotherapeutic agent. For example, a compound of Formulae I-A, I-B, I-C, I-D, I-E, I-F, and I-G as defined herein, and an additional therapeutic agent can be administered in a single formulation. Other combinations are also encompassed by combination therapy. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or weeks of each other. In some cases, even longer intervals are possible. While in many cases it is desirable that the two or more agents used in a combination therapy be present in within the patient's body at the same time, this need not be so.

Combination therapy can also include two or more administrations of one or more of the agents used in the combination using different sequencing of the component agents. For example, if agent X and agent Y are used in a combination, one could administer them sequentially in any combination one or more times, e.g., in the order X—Y—X, X—X—Y, Y—X—Y, Y—Y—X, X—X—Y—Y, etc.

Combination therapy can also include two or more administrations of one or more of the agents used in the combination using different routes of administration. Each of the one or more of the agents may be independently administered orally, subcutaneously, topically, parenterally, by inhalation spray or rectally in dosage unit formulations containing conventional non-toxic pharmaceutically acceptable carriers, adjuvants and vehicles. Parenteral administration may include subcutaneous injections, intravenous or intramuscular injections or infusion techniques.

In some embodiments, the compounds of Formulae I-A, I-B, I-C, I-D, I-E, I-F, and I-G as described herein, are combined with asparaginase (ASNase, L-asparaginase) or its derivatives. In some embodiments, asparaginase is obtained from Erwinia chrysanthemi and is known as crisantaspase or asparaginase Erwinia chrysanthemi. Asparaginase Erwinia chrysanthemi is sold under the trademarks Erwinaze® or Erwinase®. In some embodiments, asparaginase is obtained from Escherichia coli and is known as colaspase. Colaspase is sold under the trademarks Elspar®, Leunase®, Kidrolase®, or Spectrila® (recombinant E. coli aparaginase). Pegylated derivatives of colaspase are pegaspargase, sold under the trademark Oncaspar®, and calaspargase pegol—mnkl, sold under the trademark Asparlas®. Other asparaginase products currently in preclinical or clinical development include JZP-458 (recombinant Erwinia asparaginase), PF745 (JZP-341), eryaspase (GRASPA®), and Xoncane.

In some embodiments, the compounds of Formulae I-A, I-B, I-C, I-D, I-E, I-F, and I-G as defined herein, are combined with an immunomodulatory agent. In some embodiments, the immunomodulatory enhances the adaptive immune response. In some embodiments, the immunomodulatory enhances the activity of antigen-presenting cells. In some embodiments, the immunomodulatory agent enhances the anti-tumor activity of myeloid cells including macrophages. In some embodiments, the immunomodulatory enhances the anti-tumor activity of Natural Killer cells. In some embodiments, the immunomodulatory agent enhances the activity of effector T Cells, including cytotoxic T Cells.

In some embodiments, the one or more additional therapeutic agents that may be administered in combination with a compound provided herein can be a MAPK pathway inhibitor. Such MAPK pathway inhibitors include, for example, MEK inhibitors, ERK inhibitors, and Ras inhibitors.

Exemplary MEK inhibitors include, but are not limited to, trametinib, selumetinib, cobimetinib, binimetinib, and pharmaceutically acceptable salts thereof. Exemplary ERK inhibitors include, but are not limited to, include, but are not limited to, ulixertinib, SCH772984, LY3214996, ravoxertinib, VX-Ile, ASN-007, GDC-0994, MK-8353, ASTX-029, LTT462, KO-947, and pharmaceutically acceptable salts thereof. Exemplary Ras inhibitors include, but are not limited to, AMG-510, MRTX849, ARS-1620, ARS-3248, LY3499446, and pharmaceutically acceptable salts thereof.

In some embodiments, the additional therapeutic agents can be immunomodulatory agents including but not limited to anti-PD-1 or anti-PDL-1 therapeutics including pembrolizumab, nivolumab, pidilizumab, cemiplimab, atezolizumab, durvalumab, BMS-936559, or avelumab. In some embodiments, the additional therapeutic agents can be anti-TIM3 (anti-HAVcr2) therapeutics including but not limited to TSR-022 or MBG453, anti-LAG3 therapeutics including but not limited to relatlimab, LAG525, or TSR-033, anti-4-1BB (anti-CD37, anti-TNFRSF9), CD40 agonist therapeutics including but not limited to SGN-40, CP-870,893 or R07009789, anti-CD47 therapeutics including but not limited to Hu5F9-G4, anti-CD20 therapeutics, anti-CD38 therapeutics, STING agonists including but not limited to ADU-S100, MK-1454, ASA404, or amidobenzimidazoles. In some embodiments, the additional therapeutic agents can be anti-CTLA4 agents including ipilimumab, tremelimumab. In some embodiments, the additional therapeutic agents can be hypomethylating agents including but not limited to azacytidine or decitabine, other immunomodulatory therapeutics including but not limited to epidermal growth factor inhibitors, statins, metformin, angiotensin receptor blockers, thalidomide, lenalidomide, pomalidomide, prednisone, or dexamethasone. In some embodiments, the additional therapeutic agents can be immunotherapeutic agents including targeted therapeutic agents, cancer vaccines, and CAR-T cell therapy.

The compounds of Formulae I-A, I-B, I-C, I-D, I-E, I-F, and I-G as described herein may be administered in combination with other therapeutic agents known to treat cancers. Such other therapeutic agents include radiation therapy, anti-tubulin agents, DNA alkylating agents, DNA synthesis-inhibiting agents, DNA intercalating agents, anti-estrogen agents, anti-androgens, steroids, anti-EGFR agents, kinase inhibitors, mTOR inhibitors, PI3 kinase inhibitors, cyclin-dependent kinase inhibitors, CD4/CD6 kinase inhibitors, topoisomerase inhibitors, Histone Deacetylase (HDAC) inhibitors, DNA methylation inhibitors, anti-HER2 agents, anti-angiogenic agents, proteasome inhibitors, PARP (poly ADP ribose polymerase) inhibitors, cell cycle regulating kinase inhibitors, thalidomide, lenalidomide, pomalidomide, bortezomib, carfilzomib, ixazomib, daratumumab, daratumumab/hyaluronidase, isatuximab, dexamethasone, and antibody-drug-conjugates (ADCs).

In an embodiment, the additional therapeutic agents can be chemotherapeutic agents including but not limited to an anti-tubulin agents (for example, paclitaxel, paclitaxel protein-bound particles for injectable suspension including nab-paclitaxel, eribulin, docetaxel, ixabepilone, vincristine, auristatins, or maytansinoids), vinorelbine, DNA-alkylating agents (including cisplatin, carboplatin, oxaliplatin, cyclophosphamide, ifosfamide, temozolomide), DNA intercalating agents or DNA topoisomerase inhibitors (including anthracyclines such as doxorubicin, pegylated liposomal doxorubicin, daunorubicin, idarubicin, mitoxantrone, or epirubicin, camptothecins such as topotecan, irinotecan, or exatecan), 5-fluorouracil, capecitabine, cytarabine, decitabine, 5-aza cytadine, gemcitabine and methotrexate.

In some embodiments, the additional therapeutic agents can be kinase inhibitors including but not limited to erlotinib, gefitinib, neratinib, afatinib, osimertinib, lapatanib, crizotinib, brigatinib, ceritinib, alectinib, lorlatinib, everolimus, temsirolimus, abemaciclib, LEE011, palbociclib, cabozantinib, ripretinib, sunitinib, pazopanib, sorafenib, regorafenib, sunitinib, axitinib, dasatinib, imatinib, nilotinib, idelalisib, ibrutinib, BLU-667, Loxo 292, larotrectinib, and quizartinib.

In some embodiments, the additional therapeutic agents can be anti-estrogen agents including but not limited to tamoxifen, fulvestrant, anastrozole, letrozole, and exemestane, anti-androgen agents including but not limited to abiraterone acetate, enzalutamide, nilutamide, bicalutamide, flutamide, cyproterone acetate, steroid agents including but not limited to prednisone and dexamethasone, PARP inhibitors including but not limited to neraparib, olaparib, talazoparib, and rucaparib, topoisomerase I inhibitors including but not limited to irinotecan, camptothecin, exatecan, and topotecan, topoisomerase II inhibitors including but not limited to anthracyclines, etoposide, etoposide phosphate, and mitoxantrone, Histone Deacetylase (HDAC) inhibitors including but not limited to vorinostat, romidepsin, panobinostat, valproic acid, and belinostat, DNA methylation inhibitors including but not limited to DZNep and 5-aza-2′-deoxycytidine, proteasome inhibitors including but not limited to bortezomib and carfilzomib, biological agents including but not limited to trastuzumab, ado-trastuzumab, pertuzumab, cetuximab, and panitumumab.

In some embodiments, the additional therapeutic agents can be anti-angiogenic agents including bevacizumab, aflibercept, and AMG386.

In some embodiments, the additional therapeutic agents can be antibody-drug-conjugates (ADCs) including DM1, DM4, MMAE, MMAF, or camptothecin payloads, brentuximab vedotin and trastuzumab emtansine, radiotherapy, therapeutic vaccines including but not limited to sipuleucel-T.

In some embodiments, the additional therapeutic agent can be an autophagy inhibitor including ULK inhibitors, VPS34 inhibitors, PIKfyve inhibitors, PPT1 inhibitors, or lysosomal blocking agents. In some embodiments, the additional therapeutic agent can be DCC-3116, SAR405, SB02024, hydroxychloroquinine, chloroquine, apilimod, MRT403, and LYS05.

In some embodiments, the additional therapeutic agent is selected from a luteinizing hormone-releasing hormone (LRH) analog, including goserelin and leuprolide.

In some embodiments, the additional therapeutic agent is selected from the group consisting of selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, pemetrexed, erlotinib, dasatanib, nilotinib, decatanib, panitumumab, amrubicin, oregovomab, Lep-etu, nolatrexed, AZD 2171, batabulin, of atumtunab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, alanosine (Sdx 102), talampanel, atrasentan, XR 311, romidepsin, ADS-100380, sunitinib, 5-fluorouracil, vorinostat, etoposide, gemcitabine, doxorubicin, irinotecan, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)-ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258, 3-[5-(methylsulfonylpiperadinemethyl)-indolylj-quinolone, vatalanib, AG-013736, AVE-0005, the acetate salt of [D-Ser(tBu) 6, Azgly 10](pyro-Glu-His-Trp-Ser-Tyr-D-Ser(tBu)-Leu-Arg-Pro-Azgly-NH₂ acetate (SEQ ID NO: 3) [C₅₉H₈₄N₁₈O₁₄—(C₂H₄O₂)ₓ where x=1 to 2.4], goserelin acetate, leuprolide acetate, triptorelin pamoate, medroxyprogesterone acetate, hydroxyprogesterone caproate, megestrol acetate, raloxifene, bicalutamide, flutanide, nilutamide, megestrol acetate, CP-724714; TAK-165, HKI-272, erlotinib, lapatanib, canertinib, ABX-EGF antibody, erbitux, EKB-569, PKI-166, GW-572016, ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, irinotecan, topotecan, doxorubicin, docetaxel, vinorelbine, bevacizumab (monoclonal antibody) and erbitux, cremophor-free paclitaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMIR-3339, ZK186619, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonist, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa, ipilumumab, and mixtures thereof.

### Pharmaceutical Compositions and Kits

Another aspect of this disclosure provides pharmaceutical compositions comprising compounds as disclosed herein formulated together with a pharmaceutically acceptable carrier. In particular, the present disclosure provides pharmaceutical compositions comprising compounds as disclosed herein formulated together with one or more pharmaceutically acceptable carriers. These formulations include those suitable for oral, rectal, topical, buccal, parenteral (e.g., subcutaneous, intramuscular, intradermal, or intravenous) rectal, vaginal, or aerosol administration, although the most suitable form of administration in any given case will depend on the degree and severity of the condition being treated and on the nature of the particular compound being used. For example, disclosed compositions may be formulated as a unit dose, and/or may be formulated for oral or subcutaneous administration.

Exemplary pharmaceutical compositions may be used in the form of a pharmaceutical preparation, for example, in solid, semisolid or liquid form, which contains one or more of the compounds described herein, as an active ingredient, in admixture with an organic or inorganic carrier or excipient suitable for external, enteral or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

For preparing solid compositions such as tablets, the principal active ingredient may be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound provided herein, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof, and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Compositions for inhalation or insufflation include solutions and suspensions in pharmaceutically acceptable, aqueous or organic solvents, or mixtures thereof, and powders. Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent.

Dosage forms for transdermal administration of a subject composition include powders, sprays, ointments, pastes, creams, lotions, gels, solutions, patches and inhalants. The active component may be mixed under sterile conditions with a pharmaceutically acceptable carrier, and with any preservatives, buffers, or propellants which may be required.

The ointments, pastes, creams, and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions and compounds of the present disclosure may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions. Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions of the present disclosure suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions provided herein include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants.

In another embodiment, provided are enteral pharmaceutical formulations including a disclosed compound and an enteric material, and a pharmaceutically acceptable carrier or excipient thereof. Enteric materials refer to polymers that are substantially insoluble in the acidic environment of the stomach, and that are predominantly soluble in intestinal fluids at specific pHs. The small intestine is the part of the gastrointestinal tract (gut) between the stomach and the large intestine, and includes the duodenum, jejunum, and ileum. The pH of the duodenum is about 5.5, the pH of the jejunum is about 6.5 and the pH of the distal ileum is about 7.5.

Accordingly, enteric materials are not soluble, for example, until a pH of about 5.0, of about 5.2, of about 5.4, of about 5.6, of about 5.8, of about 6.0, of about 6.2, of about 6.4, of about 6.6, of about 6.8, of about 7.0, of about 7.2, of about 7.4, of about 7.6, of about 7.8, of about 8.0, of about 8.2, of about 8.4, of about 8.6, of about 8.8, of about 9.0, of about 9.2, of about 9.4, of about 9.6, of about 9.8, or of about 10.0. Exemplary enteric materials include cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, natural resins such as zein, shellac and copal collophorium, and several commercially available enteric dispersion systems (e.g., Eudragit L30D55, Eudragit FS30D, Eudragit L100, Eudragit S100, Kollicoat EMM30D, Estacryl 30D, Coateric, and Aquateric). The solubility of each of the above materials is either known or is readily determinable in vitro. The foregoing is a list of possible materials, but one of skill in the art with the benefit of the disclosure would recognize that it is not comprehensive and that there are other enteric materials that would meet the objectives described herein.

Advantageously, provided herein are kits for use by a e.g., a consumer in need of treatment of cancer. Such kits include a suitable dosage form such as those described above and instructions describing the method of using such dosage form to mediate, reduce or prevent inflammation. The instructions would direct the consumer or medical personnel to administer the dosage form according to administration modes known to those skilled in the art. Such kits could advantageously be packaged and sold in single or multiple kit units. An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc. . . . Second Week, Monday, Tuesday, . . . ” etc. Other variations of memory aids will be readily apparent. A “daily dose” can be a single tablet or capsule or several pills or capsules to be taken on a given day. Also, a daily dose of a first compound can consist of one tablet or capsule while a daily dose of the second compound can consist of several tablets or capsules and vice versa. The memory aid should reflect this.

### EXAMPLES

The compounds described herein can be prepared in a number of ways based on the teachings contained herein and disclosures of synthetic procedures in the art. In the description of the synthetic methods described below, it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials.

The following abbreviation are used in this disclosure and have the following definitions: “CH₃CN” is acetonitrile, “ADP” is adenosine diphosphate, “ASNase” is Asparaginase, “Boc” is t-butylcarbonate, “BSA” is bovine serum albumin, “CuCl” is copper(I) chloride, “DCC” is N,N′-Dicyclohexylcarbodiimide, “DCM” is dichloromethane, “DIEA” is N,N-diisopropylethylamine, “DMF” is N,N-dimethylformamide, “dppf” is 1,1′-bis(diphenylphosphino)ferrocene, “DMSO” is dimethylsulfoxide, “EDTA” is ethylenediaminetetraacetic acid, “ESI” is electrospray ionization, “EtOAc” is ethyl acetate, “EtOH” is ethanol, “GST” is glutathione S-transferase, “h” is hour or hours, “HATU” is 1-[bis(dimethylamino)methylene]-1H-1,2,3-triazolo[4,5-b]pyridinium 3-oxide hexafluorophosphate, “HBTU” is (2-(1H-benzotriazol-1-yl)-1,1,3,3-tetramethyluronium hexafluorophosphate, “H₂” is hydrogen gas, “HCl” is hydrochloric acid, “IPA” is isopropyl alcohol, “H₂O” is water, “HOBt” is Hydroxybenzotriazole “IC₅₀” is half maximal inhibitory concentration, “K₂CO₃” is potassium carbonate, “KOAc” is potassium acetate, “K₂PO₄” is Dipotassium phosphate, “LAH” is lithium aluminum hydride, “MeCN” is acetonitrile, “MeOH” is methanol, “MnO₂” is manganese dioxide, “MgSO₄” is magnesium sulfate, “MHz” is megahertz, “min” is minute or minutes, “MS” is mass spectrometry, “NADH” is nicotinamide adenine dinucleotide, “Na₂CO₃” is sodium carbonate, “NaHCO₃” is sodium bicarbonate, “NaNO₂” is sodium nitrite, “NaOH” is sodium hydroxide, “NaOMe” is sodium methoxide, “NaSMe” is sodium thiomethoxide, “Na₂SO₄” is sodium sulfate, “NBS” is N-bromosuccinimide, “NCS” is N-chlorosuccinimide, “NIS” is N-iodosuccinimide, “NIH₄Cl” is ammonium chloride, “NMIR” is nuclear magnetic resonance, “PBS” is phosphate buffered saline, “Pd/C” is palladium on carbon, “Pd₂(dba)₃” is tris(dibenzylideneacetone)dipalladium(O), “Pd(dppf)Cl₂” is 1,1-bis(diphenylphosphino)ferrocene-palladium(II)dichloride, “Pd(PPh₃)₄” is tetrakis(triphenylphosphine)palladium, “POCl₃” is phosphorus oxychloride, “rt” is room temperature which is also known as “ambient temp,” which will be understood to consist of a range of normal laboratory temperatures ranging from 15-25° C., “sat'd.” is saturated, “NaBH₄” is sodium borohydride, “SM” is starting material, “SOCl₂” is thionyl chloride, “TEA” is triethylamine, “THF” is tetrahydrofuran, “Xantphos” is 4,5-bis(diphenylphosphino)-9,9-dimethylxanthene.

### General Chemistry

Exemplary compounds described herein are available by the general synthetic methods illustrated in the Schemes below, intermediate preparations, and the accompanying Examples.

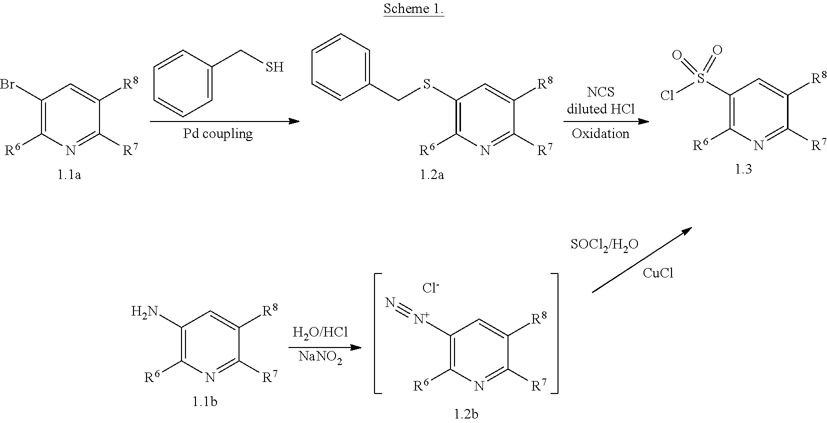

Scheme 1 illustrates an exemplary preparation of sulfonyl chlorides 1.3. Bromides 1.1a (commercially available or synthesized by those skilled in the art) are converted to thio-ethers 1.2a by a Pd catalyzed coupling reaction (for example, using Pd₂(dba)₃, XantPhos, phenylmethanthiol in the presence of a base, such as DIEA in a solvent such as toluene, and at elevated temperature). A smooth oxidation of thio-ethers 1.2a (e.g., by combination of NCS and dilute HCl, 1,3-dichloro-5,5-dimethylhydantoin) affords the corresponding sulfonyl chlorides 1.3 according to general reaction conditions reported in Synthesis, 2006, 24, 4131-4134 and Bioorg. Med. Chem., 2017, 25, 3447-3460. Alternatively, sulfonyl chlorides 1.3 can be prepared from amines 1.1b (commercially available or synthesized by those skilled in the art) by diazotization, followed by Cu-mediated chlorination of the resulting intermediate (according to general reaction conditions reported in Org. Proc. Res. Dev., 2009, 5, 875-879).

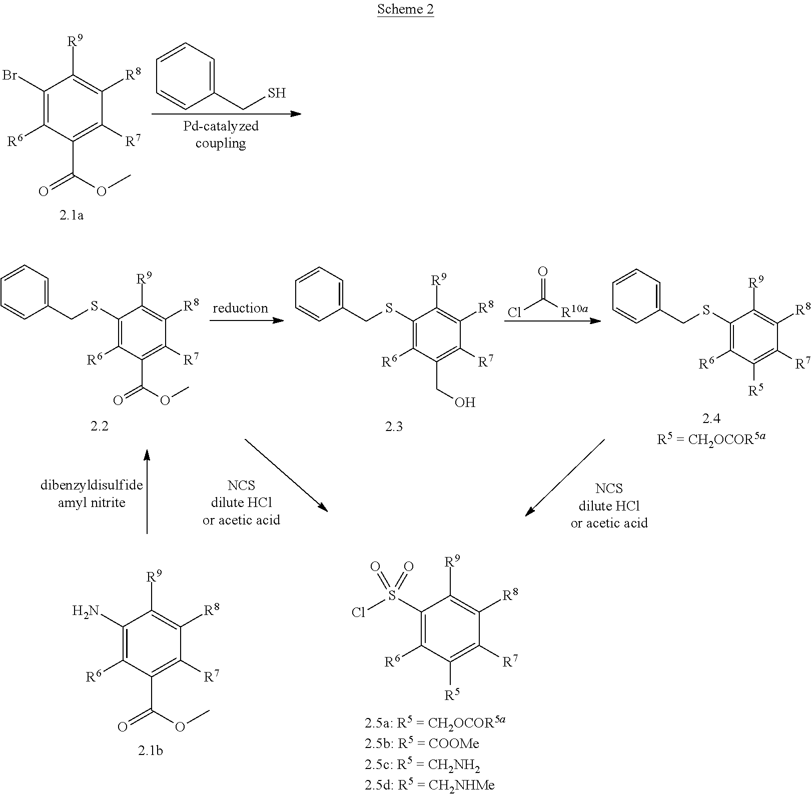

Scheme 2 illustrates an exemplary preparation of sulfonyl chlorides 2.5. Bromides 2.1a (commercially available or synthesized by those skilled in the art) are converted to thio-ethers 2.2 a Pd catalyzed coupling reaction, for example, using Pd₂(dba)₃, XantPhos, phenylmethanthiol in the presence of a base, such as DIEA in an aprotic solvent such as toluene, and at elevated temperature. Alternatively, compounds 2.2 can be prepared from anilines 2.1b (commercially available or synthesized by those skilled in the art) by diazotization using dibenzyldisulfide and amyl nitrite in CH₃CN at elevated temperature. Reduction of esters 2.2 with LAH affords primary alcohols 2.3. Acylation of alcohols 2.3 with acyl chloride R^{5a}—COCl in a presence of a base such as DIEA affords 2.4. Finally, A smooth oxidation of thio-ethers 2.4 (R⁵═CH₂OCOR^{5a}) by combination of NCS and dilute HCl or acetic acid affords the corresponding sulfonyl chlorides 2.5 according to general reaction conditions reported in Synthesis, 2006, 24, 4131-4134 and Bioorg. Med. Chem., 2017, 25, 3447-3460. In another embodiment, 2.5b (R⁵═COOMe) can be prepared from thio-ethers 2.2 by combination of NCS and dilute HCl or acetic acid.

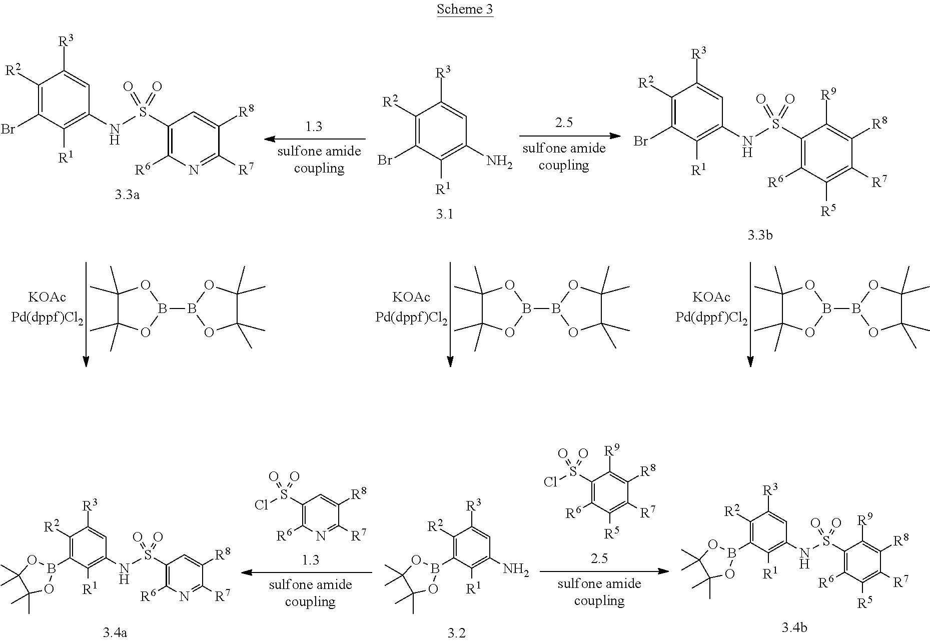

Scheme 3 illustrates an exemplary preparation of boronates 3.4a and 3.4b. Compounds 3.1 (commercially available, synthesized as described in WO2013134298 or synthesized by those skilled in the art) react with bis(pinacolato)diboron by borylation reaction known to those skilled in the art (palladium-mediated reaction conducted using palladium catalysts such as Pd(dppf)Cl₂, a suitable base such as KOAc in a suitable solvent such as 1,4-dioxane at elevated temperature) to obtain compounds 3.2 which are reacted with sulfonyl chlorides 1.3 and 2.5 to give compounds 3.4a and 3.4b respectively. Alternatively, compounds 3.1 react with sulfonyl chlorides 1.3 and 2.5 to afford the sulfonamides 3.3a and 3.3b which are converted to the boronates 3.4a and 3.4b respectively under the borylation reaction conditions known to those skilled in the art.

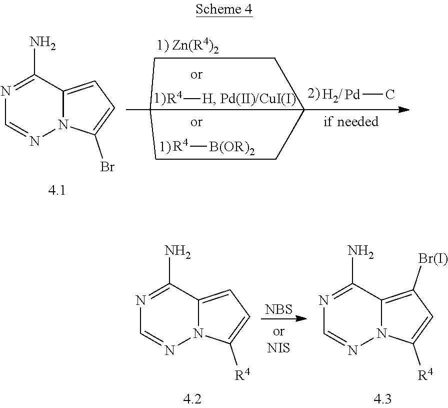

Scheme 4 illustrates an exemplary preparation of intermediates 4.3. Commercially available 7-bromopyrrolo[2,1-f][1,2,4]triazin-4-amine 4.1 converts to 4.2 by several different methods: (1) Pd-catalyzed coupling reaction with Zn(R⁴)₂ (2) Pd(II)/Cu(I) mediated Sonogashira coupling with R⁴—H and (3) Suzuki reaction with R⁴—B(OR)₂. If compounds 4.2 contain double bond or triple bond, reduction can be occurred under hydrogen gas atmosphere in the presence of catalyst such as Pd—C. Finally, bromination (or iodination) of 4.2 with NBS (or NIS) affords intermediates 4.3.

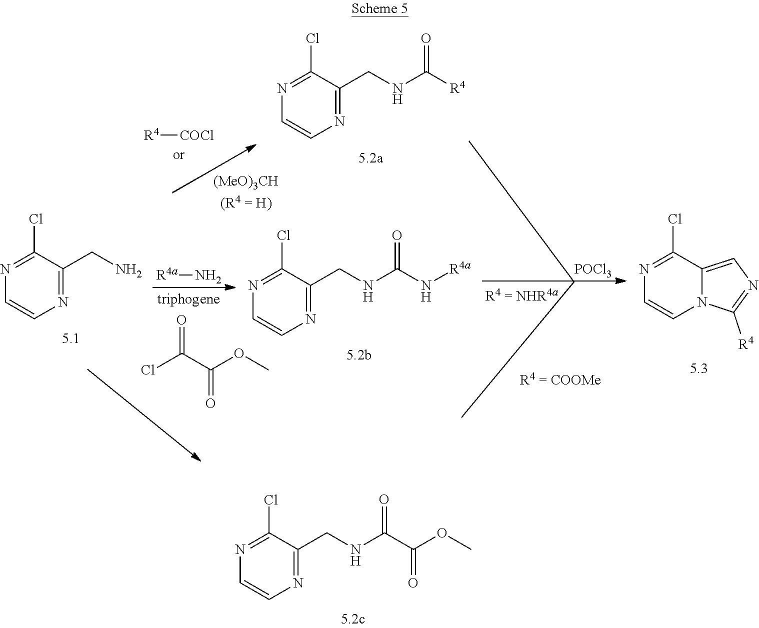

Scheme 5 illustrates an exemplary preparation of intermediates 5.3. Amide formation of (3-chloropyrazin-2-yl)methanamine 5.1 with acyl chlorides (R⁴—COCl) or trimethyl orthoformate (R⁴═H) under proper conditions by those skilled in the art affords amides 5.2a. Urea formation of 5.1 with amines (R^{4a}—NH₂) under triphosgene condition affords urea 5.2b (R⁴═NHR^{4a}). Reaction of 5.1 with methyl 2-chloro-2-oxoacetate affords methyl 2-(((3-chloropyrazin-2-yl)methyl)amino)-2-oxoacetate 5.2c. Cyclisation of amides 5.2a, 5.2b, and 5.2c with POCl₃ in an aprotic solvent such as CH₃CN at elevated temperature affords 3-substituted-8-chloroimidazo[1,5-a]pyrazines 5.3.

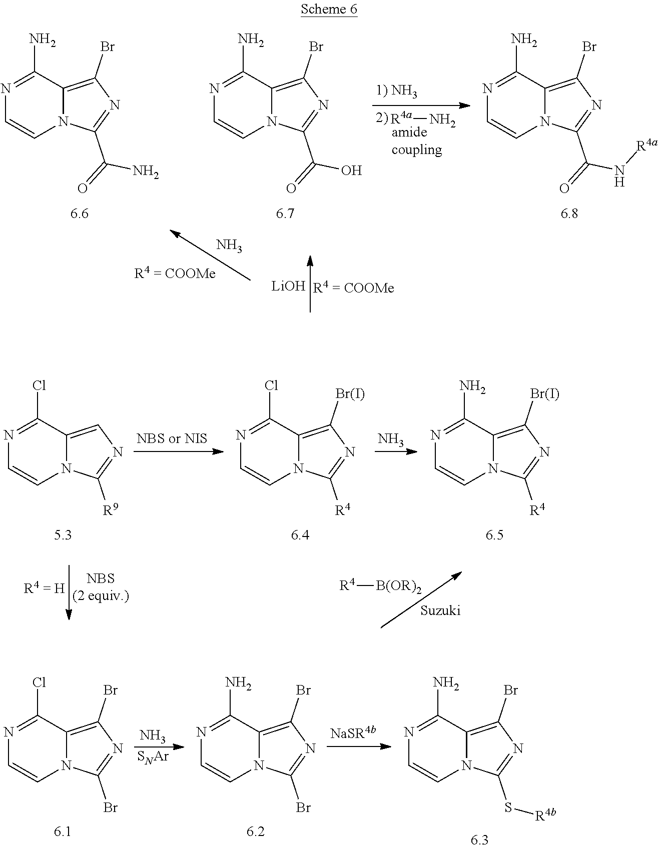

Scheme 6 illustrates an exemplary preparation of intermediates 6.3, 6.5, 6.6, and 6.8. Bromination (2 equiv.) of 8-chloroimidazo[1,5-a]pyrazine (5.3: R⁴═H) in DMF affords 1,3-dibromo-8-chloroimidazo[1,5-a]pyrazine (6.1). Substitution of 6.1 with ammonia in a protic solvent such as CH₃CN at elevated temperature in a sealed tube affords 1,3-dibromoimidazo[1,5-a]pyrazin-8-amine (6.2). Substitution of 6.2 with NaSR^{4b} gives thioethers 6.3. Pd-catalyst coupling reaction of 6.2 with boronates R⁴—B(OR)₂ under Suzuki conditions affords intermediates 6.5. In another embodiment, bromination (or iodination) of compounds 5.3 with NBS (or NIS) in DMF affords compounds 6.4. Substitution of 6.4 with ammonia in a protic solvent such as IPA at elevated temperature in a sealed tube affords intermediates 6.5 (from Cl to NH₂). Methyl 1-bromo-8-chloroimidazo[1,5-a]pyrazine-3-carboxylate (6.4: R⁴═COOMe) reacts with ammonia in CH₃CN at elevated temperature in a sealed tube to afford 8-amino-1-bromoimidazo[1,5-a]pyrazine-3-carboxamide (6.6). Hydrolysis of 6.4 (R⁴═COOMe) with LiOH affords 1-bromo-8-chloroimidazo[1,5-a]pyrazine-3-carboxylic acid (6.7). Substitution reaction of 6.7 with ammonia in a protic solvent at elevated temperature in a sealed tube affords 8-amino-1-bromoimidazo[1,5-a]pyrazine-3-carboxylic acid which can be reacted with amines R^{4a}—NH₂ under amide coupling reaction known to those skilled in the art affords amides 6.8.

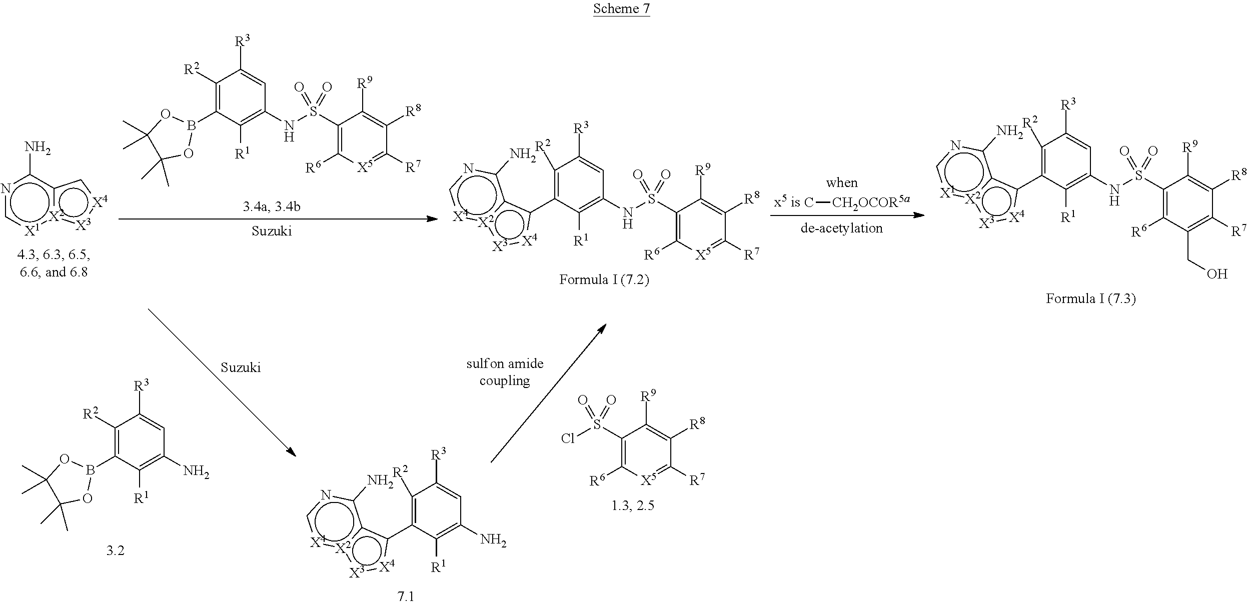

Scheme 7 illustrates an exemplary preparation of Formula I. Bromides (or iodides) 4.3, 6.3, 6.5, 6.6, 6.7, and 6.8 react with boronates 3.2 in a presence of a Pd catalyst (Suzuki conditions) to afford anilines 7.1. Sulfone amide coupling reaction of anilines 7.1 with sulfonyl chlorides 1.3, and 2.5 affords Formula I (7.2). Alternatively, Formula I (7.2) can be prepared from bromides (or iodides) 4.3, 6.3, 6.5, 6.6, 6.7 and 6.8 with boronates 3.4a, and 3.4b under Suzuki conditions. When X⁵ is C—CH₂OCOR^{5a}, hydrolysis (de-acetylation) of Formula I (7.2) with K₂CO₃ affords free hydroxy compound Formula I (7.3).

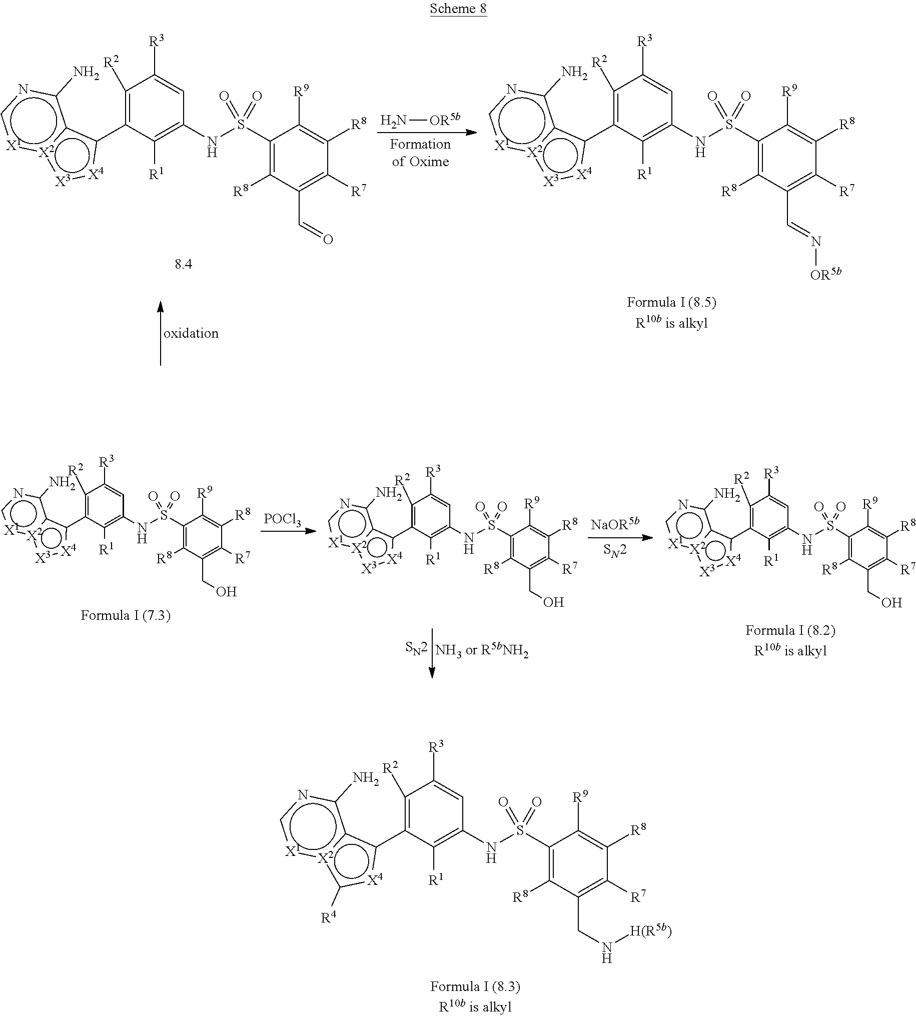

Scheme 8 illustrates an exemplary preparation of Formula I (8.2, 8.3, and 8.5). Compounds Formula I (7.3) react with POCl₃ to afford chlorides 8.1. Substitution reaction of chlorides 8.1 with nucleophiles, NaOR^{5b}, NH₃/MeOH, or R^{5b}—NH₂ affords Formula I (8.2, and 8.3) respectively. In another embodiment, oxidation of Formula I (7.3) with oxidation reagents such as MnO₂ affords the corresponding aldehydes 8.4. Aldehydes 8.4 can be converted to substituted oximes Formula I (8.5).

### Preparation of Intermediates and Final Compounds

Using the synthetic procedures and methods described herein and methods known to those skilled in the art, the following compounds were made:

### Pd-Borylation

### Preparation of Example A1: 2-fluoro-3-(4,4,5,5-tetramethyl-1,3,2-dioxaborolan-2-yl)aniline

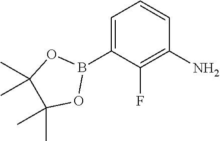 [SMILES: CC1(C)OB(c2cccc(N)c2F)OC1(C)C]

A solution of 3-bromo-2-fluoroaniline (2.5 g, 13 mmol), pinacol diborane (4.3 g, 17 mmol) and KOAc (9.9 g, 101 mmol) in 1,4-dioxane (45 mL) was degassed with Ar for 10 min. PdCl₂(dppf) (0.48 g, 0.66 mmol) was added and the reaction mixture was degassed with Ar for an additional 5 min. The reaction mixture was heated to 100° C. for 1 h. The reaction mixture was filtered through a pad of celite and washed with 1,4-dioxane. The filtrate was concentrated under reduced pressure and the crude was purified by silica gel column chromatography (0 to 100% EtOAc/hexanes) to obtain 2-fluoro-3-(4,4,5,5-tetramethyl-1,3,2-dioxaborolan-2-yl)aniline (2.5 g, 80%) as a brown oil. ¹H NMR (400 MHz, DMSO-d₆): δ 6.86 (m, 2H), 6.76 (d, J=6.8 Hz, 1H), 5.02 (s, 2H), 1.28 (s, 12H); MS (ESI) m/z: 238.2 (M+H⁺).

The following compounds are prepared essentially by method of preparation A1.

| Ex | | Yield | ¹H NMR | MS (m/z: |
|---|---|---|---|---|
| No | Product | (%) | (400 MHz, DMSO-d₆): δ | M + H⁺) |
| A2 | 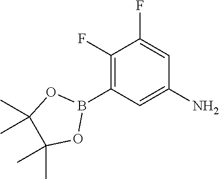 [SMILES: CC1(C)OB(c2cc(N)cc(F)c2F)OC1(C)C] | 36 | 6.59 (m, 2H), 5.20 (br s, 2H), 1.28 (s, 12H). | 256.1 |
| A3 | 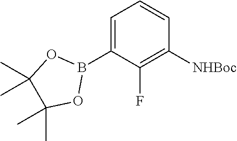 [SMILES: CC(C)(C)OC(=O)Nc1cccc(B2OC(C)(C)C(C)(C)O2)c1F] | 78 | 8.87 (s, 1H), 7.34 (m, 1H), 7.33 (t, J = 5.6 Hz, 1H), 7.13 (m, 1H), 1.45 (s, 9H), 1.29 (s, 12H). | No Data |

### Formation of Thio-Ether

### Preparation of example B1: ethyl 3-(benzylthio)-2,5-dichlorobenzoate

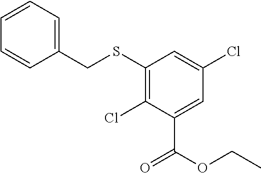 [SMILES: CCOC(=O)c1cc(Cl)cc(SCc2ccccc2)c1Cl]

A solution of ethyl 3-amino-2,5-dichlorobenzoate (12.0 g, 51 mmol) in CH₃CN (250 mL) was treated with amyl nitrite (9.6 mL, 81 mmol). Dibenzyl disulfide (12.6 g, 51 mmol) was added at rt and the reaction mixture was heated at 70° C. for 3 h. The reaction mixture was quenched with ice water (100 mL) and extracted with EtOAc (3×). The combined organics were dried over anhydrous Na₂SO₄, filtered, and concentrated under reduced pressure. The crude was purified by silica gel column chromatography (10 to 20% EtOAc/hexanes) to afford ethyl 3-(benzylthio)-2,5-dichlorobenzoate (10.0 g, 58%) as an off-white solid. ¹H NMR (400 MHz, DMSO-d₆): δ 7.63 (d, J=2.8 Hz, 1H), 7.56 (d, J=2.4 Hz, 1H), 7.44 (d, J=7.2 Hz, 2H), 7.35 (t, J=7.6 Hz, 2H) 7.28 (d, J=7.2 Hz, 1H), 4.42 (s, 2H), 4.33 (q, J=7.2 Hz, 2H), 1.29 (t, J=6.8 Hz, 3H).

The following compounds are prepared essentially by method of preparation B1.

| Ex | | Yield | ¹H NMR |
|---|---|---|---|
| No | Product | (%) | (400 MHz, DMSO-d₆): δ |
| B2 | 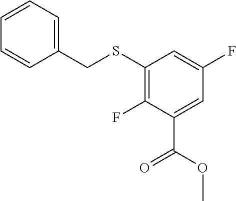 [SMILES: COC(=O)c1cc(F)cc(SCc2ccccc2)c1F] | 41 | 7.59-7.72 (m, 1H), 7.48 (m, 1H), 7.38-7.44 (m, 2H), 7.20-7.26 (m, 2H), 7.15 (m, 1H), 4.03 (s, 2H), 3.77 (s, 3H). |
| B3 | 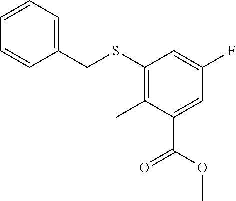 [SMILES: COC(=O)c1cc(F)cc(SCc2ccccc2)c1C] | 24 | 7.39 (m, 3H), 7.24-7.34 (m, 4H), 4.32 (s, 2H), 3.81 (s, 3H), 2.32 (s, 3H). |
| B4 | 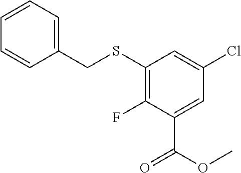 [SMILES: COC(=O)c1cc(Cl)cc(SCc2ccccc2)c1F] | 50 | 7.72 (m, 1H), 7.63 (m, 1H), 7.36 (d, J = 7.2 Hz, 2H), 7.33 (t, J = 6.8 Hz, 2H), 7.26 (m, 1H), 4.37 (s, 2H), 3.85 (s, 3H). |
| B5 | 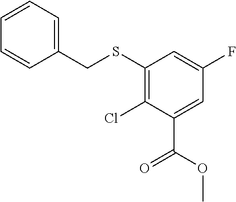 [SMILES: COC(=O)c1cc(F)cc(SCc2ccccc2)c1Cl] | 45 | 7.46-7.52 (m, 3H), 7.29-7.44 (m, 4H), 4.39 (s, 2H), 3.85 (s, 3H). |

### Formation of Thio-ether

### Preparation of example B6: methyl 3-(benzylthio)-5-chloro-2-methoxybenzoate

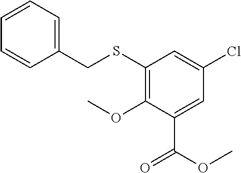 [SMILES: COC(=O)c1cc(Cl)cc(SCc2ccccc2)c1OC]

A solution of methyl 3-bromo-5-chloro-2-methoxybenzoate (1.0 g, 3.6 mmol) and phenylmethanethiol (0.50 g, 4.3 mmol) in toluene (10 mL) was treated with DIEA (1.3 mL, 7.2 mmol). The mixture was purged with Ar for 5 min and then XantPhos (0.18 g, 0.32 mmol) and Pd₂(dba)₃ (0.16 g, 0.17 mmol) were added. The mixture was purged with Ar again for 5 min and heated to 90° C. overnight. The reaction was cooled to rt, and filtered through a pad of silica gel, washed with EtOAc:hexanes (1:1). The filtrate was concentrated under reduced pressure and the residue was purified by silica gel column chromatography (0 to 100% EtOAc/hexanes) to obtain methyl 3-(benzylthio)-5-chloro-2-methoxybenzoate (0.81 g, 74%) as an orange oil. ¹H NMR (400 MHz, DMSO-d₆): δ 7.55 (d, J=2.4 Hz, 1H), 7.47 (d, J=2.4 Hz, 1H), 7.41 (m, 2H), 7.33 (m, 2H), 7.24 (m, 1H), 4.14 (s, 2H), 3.90 (s, 3H), 3.70 (s, 3H).

The following compounds are prepared essentially by method of preparation B6.

| Ex No | Product | Yield (%) | ¹H NMR (400 MHz, DMSO-d₆): δ |
|---|---|---|---|
| B7 | 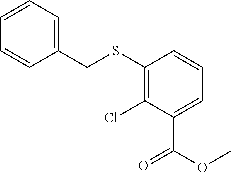 [SMILES: COC(=O)c1cccc(SCc2ccccc2)c1Cl] | 80 | 7.60 (dd, J = 1.5, 8.0 Hz, 1H), 7.50 (dd, J = 1.5, 7.6 Hz, 1H), 7.37-7.46 (m, 2H), 7.31-7.37 (m, 2H), 7.23-7.31 (m, 2H), 4.34 (s, 2H), 3.85 (s, 3H). |
| B8 | 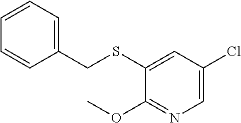 [SMILES: COc1ncc(Cl)cc1SCc1ccccc1] | 100 | 7.94 (d, J = 2.4 Hz, 1 H), 7.65 (d, J = 2.6 Hz, 1H), 7.41 (d, J = 7.9 Hz, 2H), 7.32 (t, J = 7.6 Hz, 2H), 7.26 (d, J = 8.3 Hz, 1H), 4.27 (s, 2H), 3.89 (s, 3H). |

### Reduction: of Ester

### Preparation of example C1: (3-(benzylthio)-2,5-dichlorophenyl)methanol

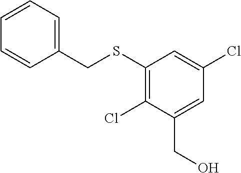 [SMILES: OCc1cc(Cl)cc(SCc2ccccc2)c1Cl]

A mixture of CaCl₂ (0.45 g, 0.92 mmol) and NaBH₄ (6.2 g, 37 mmol) in EtOH (200 mL) was stirred at 0° C. A cold solution of ethyl 3-(benzylthio)-2,5-dichlorobenzoate (B1, 14 g, 9.2 mmol) in THF (200 mL) was added in drop-wise manner at 0° C. The reaction mixture was slowly warmed to rt, and then heated at 60° C. for 5 h. The reaction mixture was cooled to rt, quenched with sat'd NH₄Cl solution (50 mL), and then extracted with EtOAc (3×). The combined organic extracts were dried over anhydrous Na₂SO₄, filtered, and concentrated under reduced pressure. The crude was purified by silica gel column chromatography (0 to 10% EtOAc/hexanes) to afford (3-(benzylthio)-2,5-dichlorophenyl)methanol (8.0 g, 66%) as a brown solid. ¹H NMR (400 MHz, DMSO-d₆): δ 7.44 (m, 2H), 7.35 (m, 3H), 7.25-7.32 (m, 2H), 5.57 (t, J=5.6 Hz, 1H), 4.51 (d, J=6.0 Hz, 2H), 4.36 (s, 2H).

The following compounds are prepared essentially by method of preparation C1.

| Ex | | Yield | ¹H NMR |
|---|---|---|---|
| No | Product | (%) | (400 MHz, DMSO-d₆): δ |
| C2 | 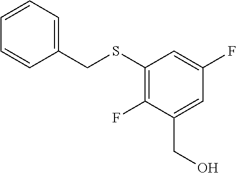 [SMILES: OCc1cc(F)cc(SCc2ccccc2)c1F] | 61 | 7.37 (m, 2H), 7.31 (m, 2H), 7.23 (m, 1H), 7.12 (m, 1H), 7.03 (m, 1H), 5.41 (t, J = 6.0 Hz, 1H), 4.50 (d, J = 5.6 Hz, 2H), 4.29 (s, 2H) |
| C3 | 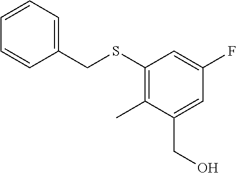 [SMILES: Cc1c(CO)cc(F)cc1SCc1ccccc1] | 69 | 7.40 (m, 2H), 7.32 (t, J = 7.2 Hz, 2H), 7.27 (m, 1H), 7.20 (m, 1H), 7.00 (m, 1H), 5.06 (s, 2H), 4.27 (s, 2H), 2.15 (s, 3H), 2.07 (s, 3H). |
| C4 | 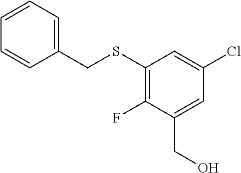 [SMILES: OCc1cc(Cl)cc(SCc2ccccc2)c1F] | 76 | 7.36 (m, 2H), 7.33 (m, 2H), 7.23-7.29 (m, 3H), 5.15 (t, J = 6.0 Hz, 1H), 4.50 (d, J = 5.6 Hz, 2H), 4.30 (s, 2H). |
| C5 | 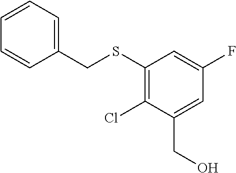 [SMILES: OCc1cc(F)cc(SCc2ccccc2)c1Cl] | 69 | 7.44 (m, 2H), 7.34-7.39 (m, 2H), 7.27 (m, 1H), 7.20 (m, 1H), 7.09 (m, 1H), 5.57 (t, J = 5.6 Hz, 1H), 4.50 (d, J = 5.6 Hz, 2H), 4.34 (s, 2H). |
| C6 | 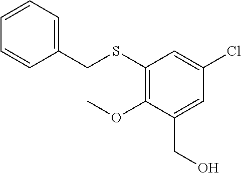 [SMILES: COc1c(CO)cc(Cl)cc1SCc1ccccc1] | 98 | 7.36 (m, 2H), 7.32 (m, 2H), 7.26 (m, 3H), 5.28 (t, J = 5.2 Hz, 1H), 4.46 (d, J = 5.2 Hz, 2H), 4.23 (s, 2H), 2.12 (s, 3H). |

### Acylation

### Preparation of example D1: 3-(benzylthio)-2,5-dichlorobenzyl acetate

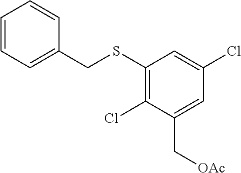 [SMILES: CC(=O)OCc1cc(Cl)cc(SCc2ccccc2)c1Cl]

A solution of (3-(benzylthio)-2,5-dichlorophenyl)methanol (C1, 0.82 g, 2.7 mmol) in THF (10 mL) was treated with acetic anhydride (0.31 mL, 3.3 mmol) at rt. The mixture was stirred at 50° C. under Ar for 2 h and then diluted with water. The solution was extracted with EtOAc (3×) and the combined organics were dried over anhydrous Na₂SO₄, filtered, and concentrated under reduced pressure to obtain 3-(benzylthio)-2,5-dichlorobenzyl acetate (0.92 g, 98%) as a white solid. ¹H NMR (400 MHz, DMSO-d₆): δ 7.51 (m, 3H), 7.41 (m, 3H), 7.35 (m, 1H), 5.16 (s, 2H), 4.45 (s, 2H), 2.16 (s, 3H).

The following compounds are prepared essentially by method of preparation D1.

| Ex No | Product | Yield (%) | ¹H NMR (400 MHz, DMSO-d₆): δ |
|---|---|---|---|
| D2 | 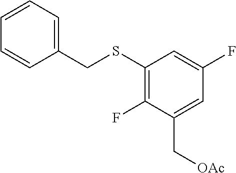 [SMILES: CC(=O)OCc1cc(F)cc(SCc2ccccc2)c1F] | 46 | 7.39 (m, 2H), 7.31 (m, 3H), 7.26 (m, 1H), 7.12 (m, 1H), 5.06 (s, 2H), 4.32 (s, 2H), 2.06 (s, 3H). |
| D3 | 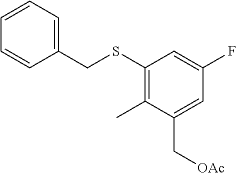 [SMILES: CC(=O)OCc1cc(F)cc(SCc2ccccc2)c1C] | 63 | 7.33-7.39 (m, 2H), 7.25-7.32 (m, 2H), 7.17-7.22 (m, 1H), 7.15 (m, 1H), 7.00 (m, 1H), 5.06 (s, 2H), 4.27 (s, 2H), 2.15 (s, 3H), 2.07 (s, 3H). |
| D4 | 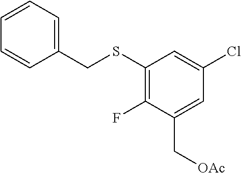 [SMILES: CC(=O)OCc1cc(Cl)cc(SCc2ccccc2)c1F] | 70 | 7.48 (dd, J = 2.4, 5.6 Hz, 1H), 7.38-7.29 (m, 5H), 7.27 (m, 1H), 5.05 (s, 2H), 4.33 (s, 2H), 2.06 (s, 3H). |
| D5 | 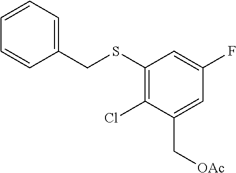 [SMILES: CC(=O)OCc1cc(F)cc(SCc2ccccc2)c1Cl] | 55 | 7.42 (m, 2H), 7.27-7.35 (m, 4H), 7.13 (m, 1H), 5.10 (s, 2H), 4.36 (s, 2H), 2.10 (s, 3H). |
| D6 | 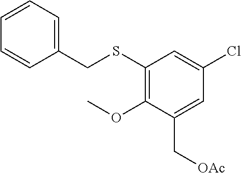 [SMILES: COc1c(COC(C)=O)cc(Cl)cc1SCc1ccccc1] | 99 | 7.40 (d, J = 7.2 Hz, 2H), 7.36 (d, J = 2.4 Hz, 1H), 7.32 (t, J = 7.2 Hz, 2H), 7.25 (m, 1H), 7.21 (d, J = 2.4 Hz, 1H), 5.03 (s, 2H), 4.29 (s, 2H), 3.71 (s, 3H), 2.00 (s, 3H). |
| D7 | 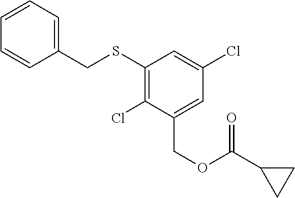 [SMILES: O=C(OCc1cc(Cl)cc(SCc2ccccc2)c1Cl)C1CC1] | 20 | 7.56 (m, 1H), 7.42-7.46 (m, 3H), 7.31-7.37 (m, 3H), 5.11 (s, 2H), 4.38 (s, 2H), 1.72 (m, 1H), 0.82-0.99 (m, 4H). |
| D8 | 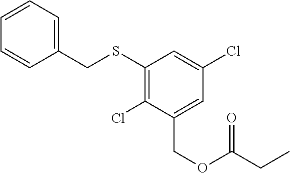 [SMILES: CCC(=O)OCc1cc(Cl)cc(SCc2ccccc2)c1Cl] | 54 | 7.45 (d, J = 2.4 Hz, 1H), 7.43 (br s, 1H), 7.26-7.34 (m, 5H), 5.12 (s, 2H), 4.39 (s, 2H), 2.40 (m, 2H), 1.04 (t, J = 7.6 Hz, 3H). |
| D9 | 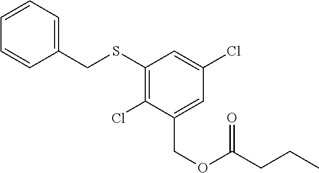 [SMILES: CCCC(=O)OCc1cc(Cl)cc(SCc2ccccc2)c1Cl] | 85 | 7.26-7.56 (m, 7H), 5.13 (s, 2H), 4.39 (s, 2H), 2.38 (t, J = 7.6 Hz, 2H), 1.53-1.60 (m, 2H), 0.88 (t, J = 5.6 Hz, 3H). |
| D10 | 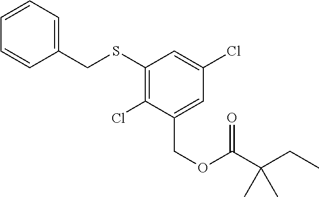 [SMILES: CCC(C)(C)C(=O)OCc1cc(Cl)cc(SCc2ccccc2)c1Cl] | 75 | 7.30-7.46 (m, 7H), 5.10 (s, 2H), 4.38 (s, 2H), 1.49 (m, 2H), 1.08 (s, 6H), 0.77 (t, J = 5.2 Hz, 3H). |

### Oxidation of Thio-Ether

### Preparation of example E1: 2,5-dichloro-3-(chlorosulfonyl)benzyl acetate

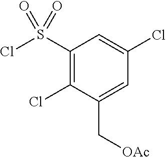 [SMILES: CC(=O)OCc1cc(Cl)cc(S(=O)(=O)Cl)c1Cl]

A solution of 3-(benzylthio)-2,5-dichlorobenzyl acetate (D1, 0.92 g, 2.7 mmol) and 1-chloropyrrolidine-2,5-dione (1.8 g, 13 mmol) in THF:H₂O (1:1, 3 mL) at 0° C. was treated with acetic acid (7 mL). The reaction mixture was slowly warmed to rt and then stirred at rt for 6 h. The reaction mixture was quenched with sat'd NaHCO₃ solution (100 mL) and extracted with EtOAc (3×). The combined organics were dried over anhydrous Na₂SO₄, filtered, and concentrated under reduced pressure. The crude was purified by silica gel column chromatography (0 to 10000 EtOAc/hexanes) to afford 2,5-dichloro-3-(chlorosulfonyl)benzyl acetate (0.45 g, 530%) as a white solid. ¹H NMR (400 MHz, DMSO-d₆): δ 7.84 (d, J=2.8 Hz, 1H), 7.52 (d, J=2.8 Hz, 1H), 5.13 (s, 2H), 2.11 (s, 3H).

The following compounds are prepared essentially by method of preparation E1.

| Ex | | Yield | ¹H NMR |
|---|---|---|---|
| No | Product | (%) | (400 MHz, DMSO-d₆): δ |
| E2 | 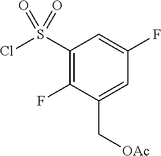 [SMILES: CC(=O)OCc1cc(F)cc(S(=O)(=O)Cl)c1F] | 59 | 7.35 (m, 1H), 7.27 (m, 1H), 5.08 (s, 2H), 2.08 (s, 3H). |
| E3 | 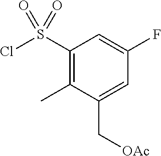 [SMILES: CC(=O)OCc1cc(F)cc(S(=O)(=O)Cl)c1C] | 72 | 7.49 (dd, J = 2.8, 9.2 Hz, 1H), 7.14 (dd, J = 2.8, 9.2 Hz, 1H), 5.07 (s, 2H), 2.44 (s, 3H), 2.08 (s, 3H). |
| E4 | 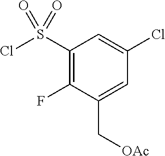 [SMILES: CC(=O)OCc1cc(Cl)cc(S(=O)(=O)Cl)c1F] | 84 | 7.60 (dd, J = 2.8, 5.6 Hz, 1H), 7.50 (dd, J = 2.8, 5.6 Hz, 1H), 5.08 (s, 2H), 2.07 (s, 3H). |
| E5 | 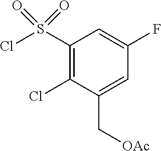 [SMILES: CC(=O)OCc1cc(F)cc(S(=O)(=O)Cl)c1Cl] | 57 | 7.61 (dd, J = 3.2, 8.4 Hz, 1H), 7.32 (dd, J = 3.2, 8.8 Hz, 1H), 5.13 (s, 2H), 2.12 (s, 3H). |
| E6 | 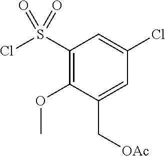 [SMILES: COc1c(COC(C)=O)cc(Cl)cc1S(=O)(=O)Cl] | 47 | No Data |
| E7 | 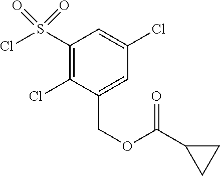 [SMILES: O=C(OCc1cc(Cl)cc(S(=O)(=O)Cl)c1Cl)C1CC1] | 86 | 7.87 (d, J = 2.7 Hz, 1H), 7.52 (d, J = 2.6 Hz, 1H), 5.16 (s, 2H), 1.75 (m, 1H), 0.84-0.98 (m, 4H). |
| E8 | 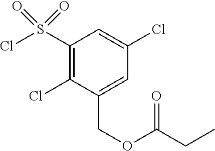 [SMILES: CCC(=O)OCc1cc(Cl)cc(S(=O)(=O)Cl)c1Cl] | 59 | 7.84 (d, J = 2.8 Hz, 1H), 7.50 (d, J = 2.4 Hz, 1H), 5.15 (s, 2H), 2.37 (m, 2H), 1.13 (t, J = 7.2 Hz, 3H). |
| E9 | 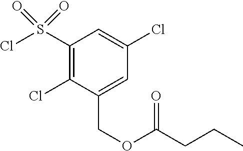 [SMILES: CCCC(=O)OCc1cc(Cl)cc(S(=O)(=O)Cl)c1Cl] | 80 | 7.84 (d, J = 2.8 Hz, 1H), 7.51 (d, J = 2.4 Hz, 1H), 5.14 (s, 2H), 2.40 (t, J = 6.4 Hz, 2H), 1.56 (q, J = 4.4 Hz, 2H), 0.90 (t, J = 5.6 Hz, 3H). |
| E10 | 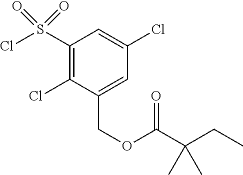 [SMILES: CCC(C)(C)C(=O)OCc1cc(Cl)cc(S(=O)(=O)Cl)c1Cl] | 80 | 7.85 (d, J = 2.8 Hz, 1H), 7.48 (d, J = 2.8 Hz, 1H), 5.11 (s, 2H), 1.46 (m, 2H), 1.05 (s, 6H), 0.83 (t, J = 5.2 Hz, 3H). |
| E11 | 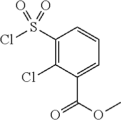 | 50 | 8.03 (dd, J = 1.8, 7.8 Hz, 1H), 7.57 (dd, J = 1.8, 7.6 Hz, 1H), 7.37 (t, J = 7.7 Hz, 1H), 3.83 (s, 3H). |
| E12 | 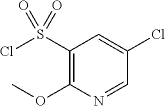 | 57 | 8.18 (d, J = 2.7 Hz, 1H), 7.93 (d, J = 2.7 Hz, 1H), 3.87 (s, 3H). |

### Sulfonylation

### Preparation of example E13: 5-chloro-2-fluoro-4-methoxybenzenesulfonyl chloride

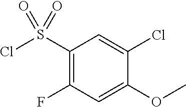

A solution of 1-chloro-4-fluoro-2-methoxybenzene (10.0 g, 62 mmol) in DCM (625 mL) was cooled to 0° C. Chlorosulfonic acid (17 mL, 250 mmol) was added and the reaction mixture was stirred at rt overnight. The reaction mixture was quenched with water and extracted with DCM (2×). The combined organics were washed with water, dried over anhydrous MgSO₄, filtered, and concentrated under reduced pressure to obtain 5-chloro-2-fluoro-4-methoxybenzenesulfonyl chloride (11.0 g, 68%) as a white crystalline solid. ¹H NMR (400 MHz, DMSO-d₆): δ 7.60 (d, J=7.4 Hz, 1H), 7.04 (d, J=11.3 Hz, 1H), 3.87 (s, 3H).

### Sulfonylation

### Preparation of example E14: methyl 2,5-dichloro-3-(chlorosulfonyl)benzoate

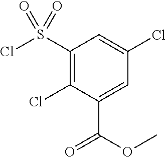 [SMILES: COC(=O)c1cc(Cl)cc(S(=O)(=O)Cl)c1Cl]

Solution A (prep: a solution of sodium nitrite (0.68 g, 9.8 mmol) in water (5.4 mL) was treated with a solution of methyl 3-amino-2,5-dichlorobenzoate (2.0 g, 9.1 mmol) in c-HCl (18 mL) at −15° C. The solution was stirred under the same conditions for 30 min.) was added dropwise into solution B (prep: thionyl chloride (2.8 mL, 39 mmol) was added dropwise into water (17 mL) under an acetone/ice bath. The mixture was stirred for 16 h at rt. Copper(I) chloride (10 mg, 1 mmol) was added at rt and then the mixture was cooled to −15° C. and stirred for 30 min.) at −15° C. The reaction mixture was stirred under the same conditions for 2 h and then diluted with DCM (100 mL). The aqueous layer was extracted with DCM (3×). The combined organic extracts were dried over anhydrous MgSO₄, filtered, and concentrated under reduced pressure. The crude was purified by silica gel column chromatography (0 to 50% EtOAc/hexanes) to obtain methyl 2,5-dichloro-3-(chlorosulfonyl)benzoate (2.1 g, 76%) as a yellow viscous liquid. ¹H NMR (400 MHz, DMSO-d₆): δ 8.00 (d, J=2.7 Hz, 1H), 7.72 (d, J=2.7 Hz, 1H), 3.86 (s, 3H).

### Formation of Sulfonamide

### Preparation of example G1: methyl 2,5-dichloro-3-(N-(2-fluoro-3-(4,4,5,5-tetramethyl-1,3,2-dioxaborolan-2-yl)phenyl)sulfamoyl)benzoate

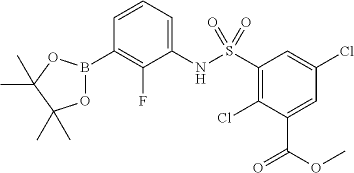

A solution of 2-fluoro-3-(4,4,5,5-tetramethyl-1,3,2-dioxaborolan-2-yl)aniline (A1, 2.2 g, 9.3 mmol) in DCM (30 mL) was treated with pyridine (2.2 mL, 28 mmol). The reaction mixture was cooled to 0° C. and a solution of methyl 2,5-dichloro-3-(chlorosulfonyl)benzoate (E14 2.8 g, 9.3 mmol) in DCM (10 mL) was added dropwise. The reaction mixture was slowly warmed to rt for 2 h. The reaction mixture was concentrated under reduced pressure and the crude was dissolved in DCM (50 mL). The solution was washed with aqueous 1.0 M HCl (2×), aqueous NaHCO₃ (3×) and brine (1×). The organic layer was dried over anhydrous Na₂SO₄, filtered, and concentrated under reduced pressure. The residue was suspended in hexanes and the solids were collected via vacuum filtration to obtain methyl 2,5-dichloro-3-(N-(2-fluoro-3-(4,4,5,5-tetramethyl-1,3,2-dioxaborolan-2-yl)phenyl)sulfamoyl)benzoate (4.2 g, 90%) as a brown solid. ¹H NMR (400 MHz, DMSO-d₆): δ 10.71 (s, 1H), 8.13 (d, J=2.6 Hz, 1H), 8.02 (d, J=2.6 Hz, 1H), 7.46 (m, 1H), 7.38 (dt, J=1.8, 7.9 Hz, 1H), 7.16 (t, J=7.7 Hz, 1H), 3.91 (s, 3H), 1.28 (s, 12H); MS (ESI) m/z 526.0 (M+Na+H⁺).

The following compounds are prepared essentially by method of preparation G1.

| Ex | | Yield | ¹H NMR | MS (m/z: |
|---|---|---|---|---|
| No | Product | (%) | (400 MHz, DMSO-d₆): δ | M + H⁺) |
| G2 | 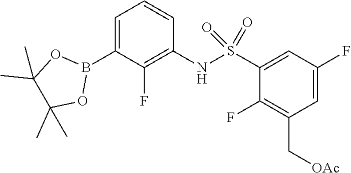 [SMILES: CC(=O)OCc1cc(F)cc(S(=O)(=O)Nc2cccc(B3OC(C)(C)C(C)(C)O3)c2F)c1F] | 86 | 10.8 (br s, 1H), 7.68 (br s, 1H), 7.51 (br s, 1H), 7.35-7.43 (br m, 2H), 7.13 (t, J = 7.6 Hz, 1H), 5.12 (s, 2H), 2.08 (s, 3H), 1.26 (s, 12H). | 402.2 (boronic acid) |
| G3 | 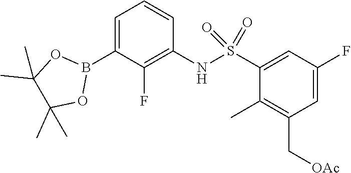 [SMILES: CC(=O)OCc1cc(F)cc(S(=O)(=O)Nc2cccc(B3OC(C)(C)C(C)(C)O3)c2F)c1C] | crude | No Data | No Data |
| G4 | 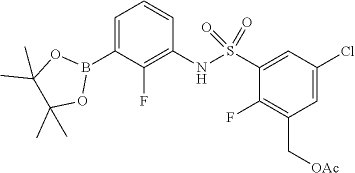 [SMILES: CC(=O)OCc1cc(Cl)cc(S(=O)(=O)Nc2cccc(B3OC(C)(C)C(C)(C)O3)c2F)c1F] | 60 | 7.88 (m, 1H), 7.66 (m, 1H), 7.45-7.51 (m, 1H), 7.38 (m, 1H), 7.15 (m, 1H), 5.14 (s, 2H), 2.07 (s, 3H), 1.26 (s, 12H). NH is missing | No Data |
| G5 | 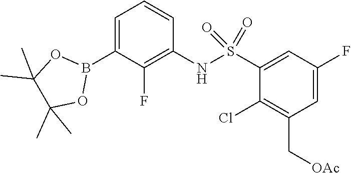 [SMILES: CC(=O)OCc1cc(F)cc(S(=O)(=O)Nc2cccc(B3OC(C)(C)C(C)(C)O3)c2F)c1Cl] | 86 | 10.61 (br s, 1H), 7.70 (m, 1H), 7.55 (t, J = 7.2 Hz, 1H), 7.43 (t, J = 5.6 Hz, 1H), 7.35 (t, J = 7.6 Hz, 1H), 7.13 (t, J = 7.6 Hz, 1H), 5.21 (s, 2H), 2.13 (s, 3H), 1.22 (s, 12H). | No Data |
| G6 | 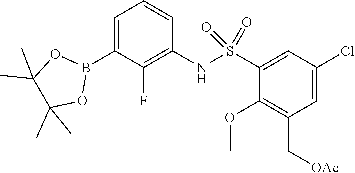 [SMILES: COc1c(COC(C)=O)cc(Cl)cc1S(=O)(=O)Nc1cccc(B2OC(C)(C)C(C)(C)O2)c1F] | 98 | 10.15 (br s, 1H), 7.74 (m, 1H), 7.64 (d, J = 2.8 Hz, 1H), 7.41 (t, J = 5.6 Hz, 1H), 7.33 (t, J = 7.6 Hz, 1H), 7.11 (t, J = 8.0 Hz, 1H), 5.14 (s, 2H), 3.84 (s, 3H), 2.10 (s, 3H), 1.27 (s, 12H). | 512.2 |
| G7 | 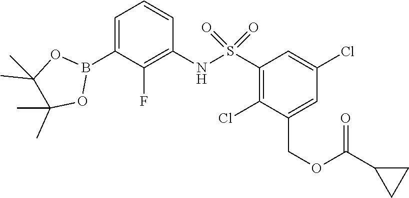 [SMILES: CC1(C)OB(c2cccc(NS(=O)(=O)c3cc(Cl)cc(COC(=O)C4CC4)c3Cl)c2F)OC1(C)C] | 73 | No Data | 544.0 |
| G8 | 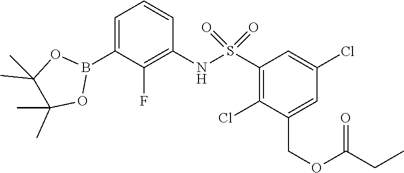 [SMILES: CCC(=O)OCc1cc(Cl)cc(S(=O)(=O)Nc2cccc(B3OC(C)(C)C(C)(C)O3)c2F)c1Cl] | 53 | No Data | 447.9 (boronic acid) |
| G9 | 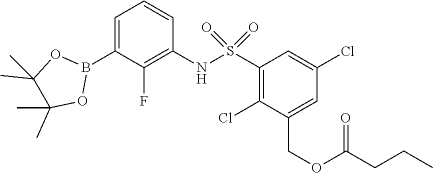 [SMILES: CCCC(=O)OCc1cc(Cl)cc(S(=O)(=O)Nc2cccc(B3OC(C)(C)C(C)(C)O3)c2F)c1Cl] | 23 | 10.8 (br s, 1H), 7.87 (d, J = 2.4 Hz, 1H), 7.73 (br s, 1H), 7.26 (t, J = 8.4 Hz, 1H), 7.07 (d, J = 9.6 Hz, 1H), 6.85 (d, J = 4.4 Hz, 1H), 5.19 (s, 2H), 2.38 (t, J = 7.2 Hz, 2H), 1.52-1.61 (m, 2H), 1.26 (s, 12H), 0.88 (d, J = 7.6 Hz, 3H). | 461.8 (boronic acid) |
| G10 | 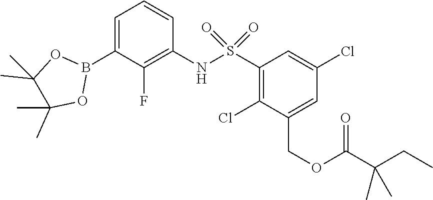 [SMILES: CCC(C)(C)C(=O)OCc1cc(Cl)cc(S(=O)(=O)Nc2cccc(B3OC(C)(C)C(C)(C)O3)c2F)c1Cl] | 50 | 10.15 (br s, 1H), 7.88 (d, J = 2.4 Hz, 1H), 7.82 (d, J = 2.0 Hz, 1H), 7.78 (m, 1H), 7.09 (br m, 1H), 6.73-6.76 (br m, 1H), 5.19 (s, 2H), 1.53 (m, 2H), 1.27 (s, 12H), 1.13 (s, 6H), 0.76 (t, J = 4.4 Hz, 3H). | 572.0 (negative) |
| G11 | 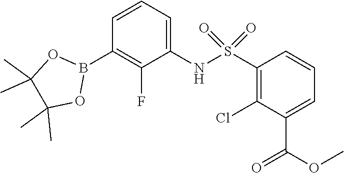 | 57 | No Data | 492.0 (M + Na + H⁺) |
| G12 | 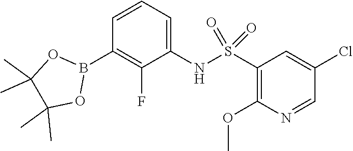 [SMILES: COc1ncc(Cl)cc1S(=O)(=O)Nc1cccc(B2OC(C)(C)C(C)(C)O2)c1F] | 94 | 10.3 (s, 1H), 8.50 (d, J = 2.5 Hz, 1H), 8.06 (d, J = 2.6 Hz, 1H), 7.45 (s, 1H), 7.39 (dd, J = 1.7, 7.7 Hz, 1H), 7.15 (t, J = 7.7 Hz, 1H), 3.89 (s, 3H), 1.27 (s, 12H). | 443.2 |
| G13 | 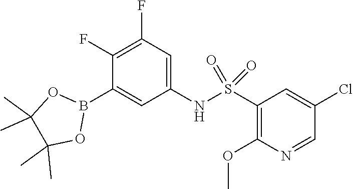 [SMILES: COc1ncc(Cl)cc1S(=O)(=O)Nc1cc(F)c(F)c(B2OC(C)(C)C(C)(C)O2)c1] | 98 | 10.7 (d, J = 2.3 Hz, 1H), 8.50 (m, 1H), 8.19 (d, J = 2.6 Hz, 1H), 7.25 (m, 1H), 7.14 (t, J = 3.0 Hz, 1H), 3.94 (s, 3H), 1.28 (s, 12H). | 461.2 |
| G14 | 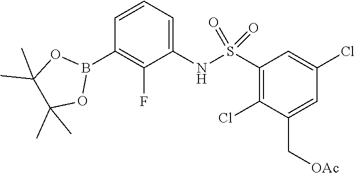 [SMILES: CC(=O)OCc1cc(Cl)cc(S(=O)(=O)Nc2cccc(B3OC(C)(C)C(C)(C)O3)c2F)c1Cl] | 77 | 10.64 (br s, 1H), 7.85 (m, 2H), 7.41 (m, 1H), 7.35 (m, 1H), 7.12 (t, J = 7.6 Hz, 1H), 5.13 (s, 2H), 2.12 (s, 3H), 1.26 (s, 12H). | 516.0 (negative) |
| G15 | 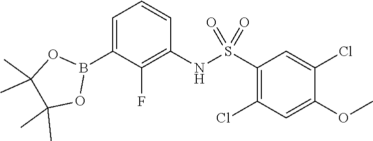 | 76 | 10.41 (s, 1H), 7.82 (s, 1H), 7.48 (s, 1H), 7.35- 7.45 (m, 2H), 7.12 (t, J = 7.8 Hz, 1H), 3.97 (s, 3H), 1.27 (s, 12H). | 498.0 (M + Na + H⁺) |
| G16 | 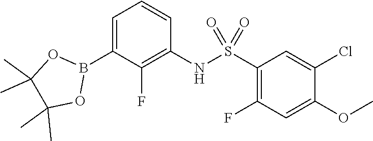 | 25 | No Data | 482.0 (M + Na + H⁺) |
| G17 | 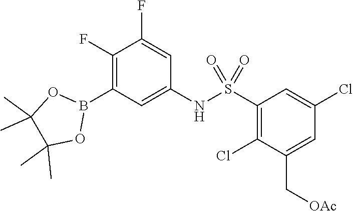 [SMILES: CC(=O)OCc1cc(Cl)cc(S(=O)(=O)Nc2cc(F)c(F)c(B3OC(C)(C)C(C)(C)O3)c2)c1Cl] | crude | 11.06 (s, 1H), 8.01 (d, J = 2.8 Hz, 1H), 7.88 (d, J = 2.8 Hz, 1H), 7.24 (m, 1H), 7.12 (br m, 1H), 5.17 (s, 2H), 2.10 (s, 3H), 1.27 (s, 12H). | 534.0 (negative) |

### Reduction of Ester

### Preparation of example G18: 2,5-dichloro-N-(2-fluoro-3-(4,4,5,5-tetramethyl-1,3,2-dioxaborolan-2-yl)phenyl)-3-(hydroxymethyl)benzenesulfonamide

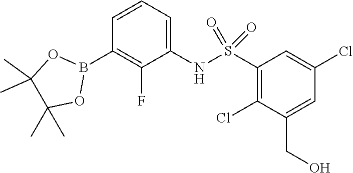 [SMILES: CC1(C)OB(c2cccc(NS(=O)(=O)c3cc(Cl)cc(CO)c3Cl)c2F)OC1(C)C]

A solution of methyl 2,5-dichloro-3-(N-(2-fluoro-3-(4,4,5,5-tetramethyl-1,3,2-dioxaborolan-2-yl)phenyl)sulfamoyl)benzoate (G1, 0.5 g, 0.99 mmol) in TIFF (5 mL) was treated with LAHI (0.11 g, 30 mmol) portion-wise at 0° C. and then stirred at 0° C. for 1 h. The reaction mixture was diluted with diethyl ether and then quenched with water (0.2 mL), 1500 aqueous NaOH (0.2 mL) and water (0.4 mL). The mixture was stirred for 4 h and then filtered through a pad of celite. The filtrate was dried over anhydrous Na₂SO₄, filtered, and concentrated under reduced pressure to obtain 2,5-dichloro-N-(2-fluoro-3-(4,4,5,5-tetramethyl-1,3,2-dioxaborolan-2-yl)phenyl)-3-(hydroxymethyl)benzenesulfonamide (0.26 g, 55%) as a colorless solid which was used for the next reaction without further purification. MS (ESI) m/z 498.0 (M+Na+H⁺).

### Acylation

### Preparation of example H1: N-((3-chloropyrazin-2-yl)methyl)acetamide

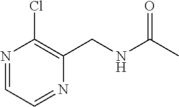

A solution of (3-chloropyrazin-2-yl)methanamine hydrochloride (5.0 g, 28 mmol) in DCM (100 mL) was cooled to 0° C. DIEA (12 mL, 69 mmol) and acetic anhydride (2.6 mL, 28 mmol) were added and the reaction mixture was stirred at 0° C. for 1 h. The reaction mixture was poured into water (100 mL) and extracted with DCM (2×). The combined organics were dried over anhydrous Mg₂SO₄, filtered, and concentrated under reduced pressure. The crude was filtered through a pad of silica gel and washed with DCM. The filtrate was concentrated under reduced pressure and the residue was treated with EtOAc/hexanes. The material was chilled under an ice bath and the solids were collected via vacuum filtration to afford N-((3-chloropyrazin-2-yl)methyl)acetamide (3.5 g, 68%) as a brown crystalline solid. ¹H NMR (400 MHz, DMSO-d₆): δ 8.64 (d, J=2.5 Hz, 1H), 8.38-8.47 (m, 2H), 4.50 (d, J=5.6 Hz, 2H), 1.90 (s, 3H).

The following compounds are prepared essentially by method of preparation H1.

| Ex | | Yield | ¹H NMR | MS (m/z: |
|---|---|---|---|---|
| No | Product | (%) | (400 MHz, DMSO-d₆): δ | M + H⁺) |
| H2 | 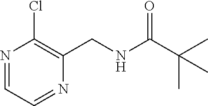 | 91 | 8.61 (d, J = 2.6 Hz, 1H), 8.41 (d, J = 2.6 Hz, 1H), 8.00 (t, J = 5.6 Hz, 1H), 4.48 (d, J = 5.6 Hz, 2H), 1.14 (s, 9H). | |
| H3 | 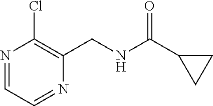 [SMILES: O=C(NCc1nccnc1Cl)C1CC1] | 58 | 8.64 (d, J = 2.4 Hz, 1H), 8.61 (t, J = 5.2 Hz, 1H), 8.43 (d, J = 2.4 Hz, 1H), 4.53 (d, J = 5.6 Hz, 2H), 1.77 (m, 1H), 1.14 (m, 2H), 1.06 (m, 2H). | 211.9 |
| H4 | 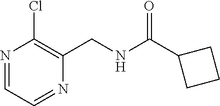 [SMILES: O=C(NCc1nccnc1Cl)C1CCC1] | 86 | 8.61 (d, J = 2.4 Hz, 1H), 8.42 (d, J = 2.4 Hz, 1H), 8.18 (t, J = 5.2 Hz, 1H), 4.49 (d, J = 5.6 Hz, 2H), 3.10 (m, 1H), 2.11-2.20 (m, 2H), 2.05 (m, 2H), 1.74-1.93 (m, 2H). | 226.2 |
| H5 | 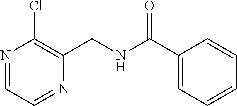 [SMILES: O=C(NCc1nccnc1Cl)c1ccccc1] | 73 | 9.05 (t, J = 5.2 Hz, 1H), 8.62 (d, J = 2.4 Hz, 1H), 8.43 (d, J = 2.0 Hz, 1H), 7.50-7.68 (m, 5H), 4.71 (d, J = 5.2 Hz, 2H). | 247.9 |
| H6 | 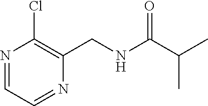 | 81 | 8.61 (d, J = 2.4 Hz, 1H), 8.42 (d, J = 2.4 Hz, 1H), 8.24 (t, J = 5.2 Hz, 1H), 4.48 (d, J = 5.6 Hz, 2H), 2.45 (m, 1H), 1.02 (d, J = 7.2 Hz, 6H). | 213.9 |
| H7 | 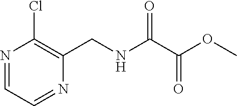 | 84 | 9.37 (m, 1H), 8.63 (d, J = 2.4 Hz, 1H), 8.45 (d, J = 2.4 Hz, 1H), 4.59 (d, J = 5.6 Hz, 2H), 3.80 (s, 3H). | 230.0 |

### Urea Formation

### Preparation of example H8: 3-((3-chloropyrazin-2-yl)methyl)-1-(4-methoxybenzyl)-1-methylurea

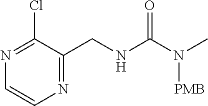

A solution of (3-chloropyrazin-2-yl) methanamine hydrochloride (4.0 g, 22 mmol) and 1-(4-methoxyphenyl)-N-methylmethanamine (5.6 g, 22 mmol) in DCM (160 mL) was cooled to 0° C. Et₃N (20 mL, 155 mmol) was added and the reaction mixture was further stirred at 0° C. for 5 min. Triphosgene (9.9 g, 33 mmol) was added portion wise under the same conditions. The reaction mixture was warmed to rt and stirred for 16 h. The reaction mixture was quenched with water (100 mL) and then extracted with DCM (2×). The combined organic extracts were dried over anhydrous Na₂SO₄, filtered, and concentrated under reduced pressure. The crude was purified by silica gel column chromatography (0 to 10% MeOH/DCM) to obtain 3-((3-chloropyrazin-2-yl)methyl)-1-(4-methoxybenzyl)-1-methylurea (2.5 g, 35%) as an off-white solid. ¹H NMR (400 MHz, DMSO-d₆): δ 8.66 (d, J=2.4 Hz, 1H), 8.41 (d, J=2.4 Hz, 1H), 7.17 (d, J=8.8 Hz, 2H), 6.98 (t, J=5.6 Hz, 1H), 6.90 (d, J=8.4 Hz, 2H), 4.48 (d, J=5.2 Hz, 2H), 4.36 (s, 2H), 3.73 (s, 3H), 2.75 (s, 3H); MS (ESI) m/z: 320.9 (M+H⁺).

The following compounds are prepared essentially by method of preparation H8.

| Ex | | Yield | ¹H NMR | MS (m/z: |
|---|---|---|---|---|
| No | Product | (%) | (400 MHz, DMSO-d₆): δ | M + H⁺) |
| H9 | 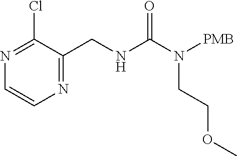 | 16 | 8.65 (d, J = 2.8 Hz, 1H), 8.41 (d, J = 2.8 Hz, 1H), 7.19 (d, J = 8.8 Hz, 2H), 6.98 (t, J = 5.6 Hz, 1H), 6.87 (d, J = 7.2 Hz, 2H), 4.48 (d, J = 5.6 Hz, 2H), 4.41 (s, 2H), 3.73 (s, 3H), 3.40 (m, 2H), 3.28 (m, 2H), 3.23 (s, 3H). | 365.0 |
| H10 | 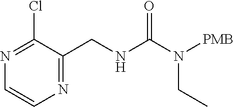 | 35 | 8.65 (d, J = 2.4 Hz, 1H), 8.41 (d, J = 2.0 Hz, 1H), 7.24 (d, J = 8.8 Hz, 2H), 6.92 (t, J = 5.6 Hz, 1H), 6.87 (d, J = 8.4 Hz, 2H), 4.47 (d, J = 5.6 Hz, 2H), 4.36 (s, 2H), 3.73 (s, 3H), 3.17 (q, J = 6.8 Hz, 2H), 1.00 (t, J = 7.2 Hz, 3H). | 334.9 |

### Cyclization

### Preparation of example I1: 8-chloro-3-methylimidazo[1,5-a]pyrazine

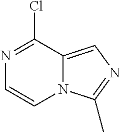

A solution of N-((3-chloropyrazin-2-yl)methyl)acetamide (H1, 3.5 g, 19 mmol) in CH₃CN (40 mL) was treated with POCl₃ (8.8 mL, 94 mmol). The reaction mixture was heated to 80° C. over the weekend. The reaction was cooled to rt and concentrated under reduced pressure. The crude was dissolved in DCM and the solution was slowly added into ice-water (100 mL) to quench residual POCl₃. The aqueous layer was extracted with DCM (3×) and the combined organics were washed with sat'd NaHCO₃, dried over anhydrous MgSO₄, filtered, and concentrated under reduced pressure. The crude was recrystallized from MeOH/EtOAc. The crystals were collected via vacuum filtration to afford 8-chloro-3-methylimidazo[1,5-a]pyrazine (1.2 g, 38%) as a brown crystalline solid. ¹H NMR (400 MHz, DMSO-d₆): δ 8.22 (dd, J=1.0, 4.9 Hz, 1H), 7.78 (d, J=1.1 Hz, 1H), 7.39 (d, J=5.0 Hz, 1H), 2.65 (s, 3H); MS (ESI) m/z: 168.2 (M+H⁺).

The following compounds are prepared essentially by method of preparation I1.

| Ex | | Yield | ¹H NMR | MS (m/z: |
|---|---|---|---|---|
| No | Product | (%) | (400 MHz, DMSO-d₆): δ | M + H⁺) |
| I2 | 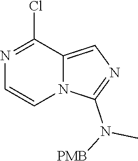 | 33 | 7.94 (dd, J = 0.8, 5.2 Hz, 1H), 7.64 (d, J = 0.8 Hz, 1H), 7.23 (d, J = 5.2 Hz, 1H), 7.18 (d, J = 8.4 Hz, 2H), 6.89 (d, J = 8.8 Hz, 2H), 4.29 (s, 2H), 3.72 (s, 3H), 2.79 (s, 3H). | 302.9 |
| I3 | 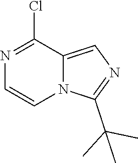 | 57 | No Data | 210.2 |
| I4 | 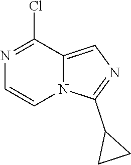 [SMILES: Clc1nccn2c(C3CC3)ncc12] | 82 | 8.42 (d, J = 4.8 Hz, 1H), 8.71 (s, 1H), 7.39 (d, J = 4.8 Hz, 1H), 2.44 (m, 1H), 1.11 (m, 2H), 1.00 (m, 2H). | 193.9 |
| I5 | 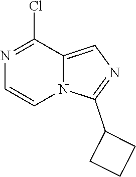 [SMILES: Clc1nccn2c(C3CCC3)ncc12] | 43 | 8.16 (d, J = 4.8 Hz, 1H), 7.82 (s, 1H), 7.36 (d, J = 5.2 Hz, 1H), 4.02 (m, 1H), 2.32-2.44 (m, 2H), 2.04-2.13 (m, 2H), 1.87-1.97 (m, 2H). | 208.2 |
| I6 | 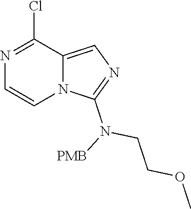 | 43 | 8.01 (d, J = 4.8 Hz, 1H), 7.64 (s, 1H), 7.24 (d, J = 5.2 Hz, 1H), 7.19 (d, J = 8.4 Hz, 2H), 6.81 (d, J = 8.8 Hz, 2H), 4.39 (s, 2H), 3.68 (s, 3H), 3.42 (m, 2H), 3.32 (m, 2H), 3.16 (s, 3H). | 347.0 |
| I7 | 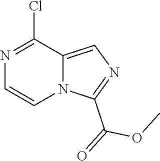 | 54 | 8.99 (d, J = 4.4 Hz, 1H), 8.13 (s, 1H), 7.78 (d, J = 5.2 Hz, 1H), 3.96 (s, 3H). | 211.8 |
| I8 | 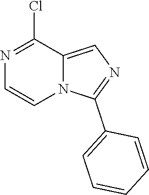 [SMILES: Clc1nccn2c(-c3ccccc3)ncc12] | 80 | 8.05 (m, 1H), 8.02 (s, 1H), 7.78-7.81 (m, 2H), 7.52-7.60 (m, 3H), 7.39 (d, J = 5.2 Hz, 1H). | 229.9 |
| I9 | 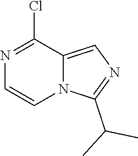 | 44 | 8.33 (d, J = 4.8 Hz, 1H), 7.79 (s, 1H), 7.37 (d, J = 5.2 Hz, 1H), 3.48-3.55 (m, 1H), 1.28 (d, J = 6.7) Hz, 6H). | 196.2 |
| I10 | 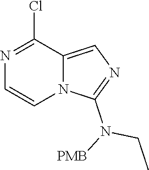 | 52 | 7.90 (d, J = 4.8 Hz, 1H), 7.67 (s, 1H), 7.24 (d, J = 5.2 Hz, 1H), 7.18 (d, J = 8.8 Hz, 2H), 6.82 (d, J = 8.4 Hz, 2H), 4.29 (s, 2H), 3.69 (s, 3H), 3.18 (q, J = 7.2 Hz, 2H), 0.99 (t, J = 6.8 Hz, 3H). | 316.9 |

### Zinc Mediated Pd-Catalyzed Coupling Reaction

### Preparation of example J1: 7-methylpyrrolo[2,1-f][1,2,4]triazin-4-amine

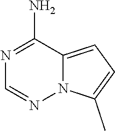

A solution of 7-bromopyrrolo[2,1-f][1,2,4]triazin-4-amine (1.2 g, 5.7 mmol) in THF (36 mL) under Ar atmosphere was treated with 2.0 M dimethyl zinc in toluene (2.8 mL, 5.7 mmol.) at rt. The reaction mixture was sparged with Ar for 10 min, then Pd(PPh₃)₄ (0.65 g, 0.57 mmol) was added. The resulting reaction mixture was heated at 60° C. for 4 h and then cooled to rt. The mixture was filtered through a pad of celite and washed with THF. The filtrate was concentrated under reduced pressure. The residue was dissolved in EtOAc (100 mL) and washed with water (2×). The organic layer was dried over anhydrous Na₂SO₄, filtered, and concentrated under reduced pressure to obtain of 7-methylpyrrolo[2,1-f][1,2,4]triazin-4-amine (0.45 g, 64%) as an off-white solid. ¹H NMR (400 MHz, DMSO-d₆): δ 7.80 (s, 1H), 7.48 (br s, 2H), 6.79 (d, J=4.4 Hz, 1H), 6.41 (d, J=4.0 Hz, 1H), 2.39 (s, 3H).

### Iodination (Bromination)

### Preparation of example K1: 8-chloro-1-iodo-3-methylimidazo[1,5-a]pyrazine

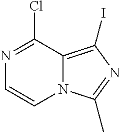

A solution of 8-chloro-3-methylimidazo[1,5-a]pyrazine (I, 1.3 g, 0.78 mmol) in DMF (15.5 mL) was treated with 1-iodopyrrolidine-2,5-dione (2.6 g, 12 mmol). The reaction mixture was heated to 60 JC overnight. The reaction was poured into DCM (200 mL) and then the solution was washed with water (2×). The combined organics were concentrated under reduced pressure and then the residue was treated with water. The resulting precipitates were filtered, rinsed with hexanes, and dried under high vacuum to afford 8-chloro-1-iodo-3-methylimidazo[1,5-a]pyrazine (2.3 g, 1010) as an orange solid. ¹H NMR (400 MHz, DM0-d₆): δ 8.26 (d, J=5.0 Hz, 1H), 7.37 (d, J=5.0 Hz, 1H), 2.61 (s, 3H); MS (ESI) m/z: 294.0 (M+H⁺).

The following compounds are prepared essentially by method of preparation K1.

| Ex | | Yield | ¹H NMR | MS (m/z: |
|---|---|---|---|---|
| No | Product | (%) | (400 MHz, DMSO-d₆): δ | M + H⁺) |
| K2 | 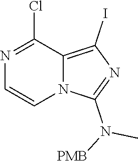 | 82 | 7.97 (d, J = 4.8 Hz, 1H), 7.21 (d, J = 5.2 Hz, 1H), 7.20 (d, J = 8.4 Hz, 2H), 6.88 (d, J = 8.4 Hz, 2H), 4.24 (s, 2H), 3.73 (s, 3H), 2.74 (s, 3H). | 428.8 |
| K3 | 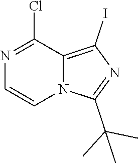 | 94 | 8.52 (d, J = 5.1 Hz, 1H), 7.30 (d, J = 5.1 Hz, 1H), 1.44 (s, 9H). | 336.0 |
| K4 | 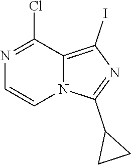 [SMILES: Clc1nccn2c(C3CC3)nc(I)c12] | 60 | 8.46 (d, J = 4.8 Hz, 1H), 7.37 (d, J = 5.2 Hz, 1H), 2.37-2.44 (m, 1H), 1.08 (m, 2H), 0.98 (m, 2H). | 319.8 |
| K5 | 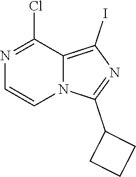 [SMILES: Clc1nccn2c(C3CCC3)nc(I)c12] | 31 | 8.19 (d, J = 4.8 Hz, 1H), 7.33 (d, J = 4.8 Hz, 1H), 3.94-4.02 (m, 1H), 2.32-2.42 (m, 2H), 2.00-2.16 (m, 2H), 1.90-1.96 (m, 2H). | 333.8 |
| K6 | 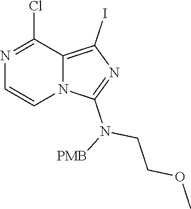 | 57 | 8.07 (d, J = 5.2 Hz, 1H), 7.23 (d, J = 5.6 Hz, 1H), 7.21 (d, J = 9.2 Hz, 2H), 6.83 (d, J = 8.8 Hz, 2H), 4.35 (s, 2H), 3.70 (s, 3H), 3.40 (m, 2H), 3.26 (m, 2H), 3.16 (s, 3H). | 472.8 |
| K7 | 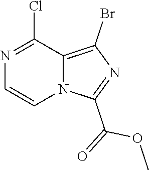 | 71 | 9.04 (d, J = 4.8 Hz, 1H), 7.78 (d, J = 4.8 Hz, 1H), 3.95 (s, 3H). | 292.0 |
| K8 | 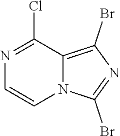 [SMILES: Clc1nccn2c(Br)nc(Br)c12] | 87 | No Data | 307.8 (negative) |
| K9 | 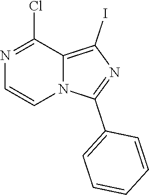 [SMILES: Clc1nccn2c(-c3ccccc3)nc(I)c12] | 59 | 8.45 (d, J = 4.8 Hz, 1H), 7.57-7.83 (m, 5H), 7.40 (d, J = 4.8 Hz, 1H). | 356.1 |
| K10 | 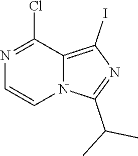 | 34 | No Data | 321.9 |
| K11 | 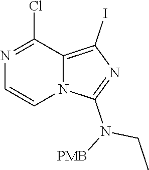 | 71 | 7.95 (d, J = 4.8 Hz, 1H), 7.22 (d, J = 5.2 Hz, 1H), 7.20 (d, J = 8.4 Hz, 2H), 6.84 (d, J = 8.4 Hz, 2H), 4.25 (s, 2H), 3.70 (s, 3H), 3.12 (q, J = 7.2 Hz, 2H), 0.98 (t, J = 7.2 Hz, 3H). | 442.8 |
| K12 | 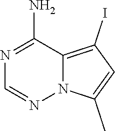 | 25 | 7.86 (s, 1H), 7.56 (br s, 2H), 6.68 (s, 1H), 2.36 (s, 3H). | No Data |

### Hydrolysis

### Preparation of example L1: 1-bromo-8-chloroimidazo[1,5-a]pyrazine-3-carboxylic acid

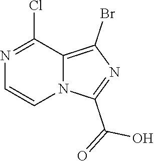 [SMILES: O=C(O)c1nc(Br)c2c(Cl)nccn12]

A solution of methyl 1-bromo-8-chloroimidazo[1,5-a]pyrazine-3-carboxylate (K7, 3.5 g, 12 mmol) in a mixture of THF and H₂O (2:1, 40 mL) was treated with LiOH monohydrate (0.99 g, 24 mmol) at rt. The reaction mixture was further stirred at rt for 2 h. The reaction mixture was concentrated under reduced pressure and the crude material was acidified with 1.0 N HCl. The precipitated solid was filtered, washed with water (5 mL), and dry under high vacuum to obtain 1-bromo-8-chloroimidazo[1,5-a]pyrazine-3-carboxylic acid (3.0 g, 97%) as an off-white solid. ¹H NMR (400 MHz, DMSO-d₆): δ 9.07 (d, J=4.8 Hz, 1H), 7.74 (d, J=4.8 Hz, 1H), acid proton is missing; MS (ESI) m/z: 275.9 (M+H⁺).

### Aromatic Substitution Reaction

### Preparation of example M1: 1-iodo-3-methylimidazo[1,5-a]pyrazin-8-amine

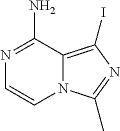

A solution of 8-chloro-1-iodo-3-methylimidazo[1,5-a]pyrazine (K1, 2.3 g, 7.8 mmol) in THF (8 mL) was treated with ammonia (2.0 M in IPA, 78 mL, 157 mmol). The vessel was sealed and heated to 110° C. 24 h. The reaction mixture was cooled to rt and concentrated under reduced pressure to afford 1-iodo-3-methylimidazo[1,5-a]pyrazin-8-amine (2.1 g, 98%) as a brown solid. ¹H NMR (400 MHz, DMSO-d₆): δ 7.59 (d, J=5.2 Hz, 1H), 7.31 (br s, 2H), 7.01 (d, J=5.2 Hz, 1H), 2.52 (s, 3H); MS (ESI) m/z: 275.0 (M+H⁺).

The following compounds are prepared essentially by method of preparation M1.

| Ex | | Yield | ¹H NMR | MS (m/z: |
|---|---|---|---|---|
| No | Product | (%) | (400 MHz, DMSO-d₆): δ | M + H⁺) |
| M2 | 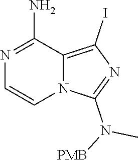 | 95 | No Data | 409.9 |
| M3 | 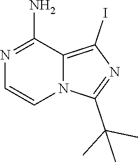 | 66 | 7.73 (d, J = 5.2 Hz, 1H), 6.89 (d, J = 5.1 Hz, 1H), 6.54 (s, 2H), 1.36 (s, 9H). | 317.0 |
| M4 | 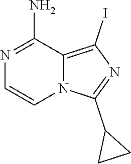 [SMILES: Nc1nccn2c(C3CC3)nc(I)c12] | 32 | No Data | 300.9 |
| M5 | 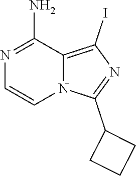 [SMILES: Nc1nccn2c(C3CCC3)nc(I)c12] | 41 | 7.46 (d, J = 5.2 Hz, 1H), 6.95 (d, J = 4.8 Hz, 1H), 6.52 (br s, 2H), 3.86 (m, 1H), 2.31-2.38 (m, 2H), 2.05 (m, 2H), 1.92-1.83 (m, 2H). | 314.8 |
| M6 | 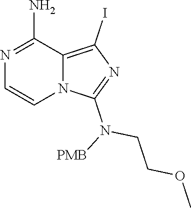 | 66 | No Data | 454.4 |
| M7 | 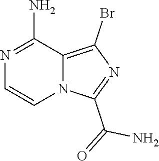 [SMILES: NC(=O)c1nc(Br)c2c(N)nccn12] | 90 | 8.59 (d, J = 4.4 Hz, 1H), 8.07 (br s, 1H), 7.71 (br s, 1H), 7.24 (d, J = 4.8 Hz, 1H), 6.87 (br s, 2H). 7.43 (d, J = 5.2 Hz, 1H), 7.13 (d, J = 5.2 Hz, 1H), 6.82 (br s, 2H). | 256.8 |
| M8 | 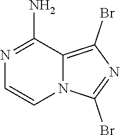 [SMILES: Nc1nccn2c(Br)nc(Br)c12] | 60 | | 292.7 |
| M9 | 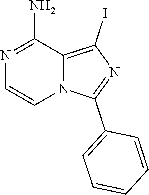 [SMILES: Nc1nccn2c(-c3ccccc3)nc(I)c12] | 26 | 7.77 (t, J = 4.4 Hz, 2H), 7.51-7.58 (br m, 2H), 7.15 (br m, 2H), 7.05 (d, J = 4.8 Hz, 1H), 6.71 (br s, 2H). | No Data |
| M10 | 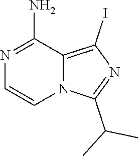 | 86 | 7.62 (d, J = 4.8 Hz, 1H), 6.97 (d, J = 4.8 Hz, 1H), 6.52 (br s, 2H), 3.37 (m, 1H), 1.25 (d, J = 6.8 Hz, 6H). | 302.7 |
| M11 | 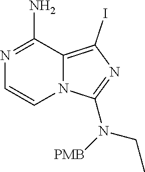 | 94 | 7.32 (d, J = 4.8 Hz, 1H), 7.19 (d, J = 8.4 Hz, 2H), 6.89 (d, J = 4.8 Hz, 1H), 6.83 (d, J = 8.4 Hz, 2H), 6.62 (br s, 2H), 4.18 (s, 2H), 3.70 (s, 3H), 3.06 (q, J = 7.2 Hz, 2H), 0.95 (t, J = 7.2 Hz, 3H). | 423.9 |
| M12 | 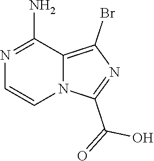 [SMILES: Nc1nccn2c(C(=O)O)nc(Br)c12] | 71 | 8.72 (d, J = 4.8 Hz, 1H), 7.04 (d, J = 4.8 Hz, 1H), 6.65 (br s, 2H), acid proton is missing. | 257.0 |

### Amide Coupling Reaction

### Preparation of example M13: 8-amino-1-bromo-N-methylimidazo[1,5-a]pyrazine-3-carboxamide

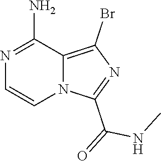

A suspension of 8-amino-1-bromoimidazo[1,5-a]pyrazine-3-carboxylic acid (M12, 0.1 g, 0.39 mmol) in DMF (4 mL) was treated with DIEA (0.6 mL, 3.9 mmol). HATU (0.50 g, 0.97 mmol) was added and the resulting mixture was stirred for 10 min at rt. A solution of methylamine hydrochloride (0.13 g, 1.95 mmol) in DMF (2 mL) was added and then the reaction mixture was stirred at rt for 3 h. The mixture was quenched with ice-cold water (5 mL) and the aqueous layer was extracted with 10% MeOH/DCM (3×). The combined organic extracts were dried over anhydrous Na₂SO₄, filtered, and concentrated under reduced pressure to obtain 8-amino-1-bromo-N-methylimidazo[1,5-a]pyrazine-3-carboxamide (0.08 g, 76%) as a light brown solid which was used for the next step without further purification. MS (ESI) m/z: 270.0 (M+H⁺) and 272.0.

### Aromatic Substitution Reaction

### Preparation of example M14: 1-bromo-3-(methylthio)imidazo[1,5-a]pyrazin-8-amine

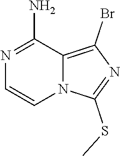

A solution of 1,3-dibromoimidazo[1,5-a]pyrazin-8-amine (L8, 0.5 g, 1.73 mmol) in DMF (5 mL) was treated with NaSMe (0.2 g, 3.46 mmol) at rt. The reaction mixture was heated at 70° C. for 16 h and then cooled to rt. The mixture was quenched with ice-cold water (20 mL). The resulting precipitates were filtered, washed with water (20 mL) and dried under high vacuum to obtain 1-bromo-3-(methylthio)imidazo[1,5-a]pyrazin-8-amine (0.3 g, 68%) as an off-white solid. ¹H NMR (400 MHz, DMSO-d₆): δ 7.47 (d, J=5.2 Hz, 1H), 7.08 (d, J=5.2 Hz, 1H), 6.74 (br s, 2H), 2.57 (s, 3H); MS (ESI) m/z: 258.8 (M+H⁺).

### Suzuki Reaction

### Preparation of example M15: 1-bromo-3-(1H-pyrazol-4-yl)imidazo[1,5-a]pyrazin-8-amine

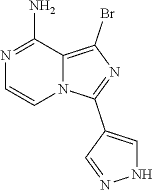 [SMILES: Nc1nccn2c(-c3cn[nH]c3)nc(Br)c12]

A solution of 1,3-dibromoimidazo[1,5-a]pyrazin-8-amine (L8, 0.50 g, 1.7 mmol) in a mixture of 1,4-dioxane:water (3:1, 10 mL) was treated with 4-(4,4,5,5-tetramethyl-1,3,2-dioxaborolan-2-yl)-1H-pyrazole (0.39 g, 2.1 mmol) and Na₂CO₃ (0.54 g, 5.2 mmol) at rt. The reaction mixture was degassed with Ar for 10 min. Pd(dppf)Cl₂DCM (0.14 g, 0.17 mmol) was added and the reaction mixture was heated at 110° C. for 4 h. The reaction mixture was cooled to rt, filtered through a pad of celite and washed thoroughly with 10% MeOH/DCM. The filtrate was washed with water and the organic layer was dried over anhydrous Na₂SO₄, filtered, and concentrated under reduced pressure to obtain the crude (0.50 g) which was used for the next step without further purification. MS (ESI) m/z: 278.9 (M+H⁺).

The following compounds are prepared essentially by method of preparation M15.

| Ex | | Yield | ¹H NMR | MS (m/z: |
|---|---|---|---|---|
| No | Product | (%) | (400 MHz, DMSO-d₆): δ | M + H⁺) |
| M16 | 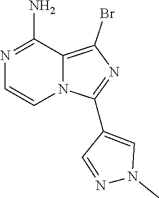 [SMILES: Cn1cc(-c2nc(Br)c3c(N)nccn23)cn1] | 30 | 8.43 (s, 1H), 7.99 (s, 1H), 7.69 (d, J = 5.2 Hz, 1H), 7.06 (d, J = 5.2 Hz, 1H), 6.71 (br s, 2H), 3.92 (s, 3H). | 292.8 |

### Suzuki Reaction

### Preparation of example N1: 5-(3-amino-2-fluorophenyl)-7-methyl-7H-pyrrolo[2,3-d]pyrimidin-4-amine

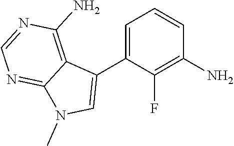 [SMILES: Cn1cc(-c2cccc(N)c2F)c2c(N)ncnc21]

A solution of 5-iodo-7-methyl-7H-pyrrolo[2,3-d]pyrimidin-4-amine (0.4 g, 1.45 mmol) in dioxane and water (3:1, 10 mL) was treated with tert-butyl (2-fluoro-3-(4,4,5,5-tetramethyl-1,3,2-dioxaborolan-2-yl)phenyl)carbamate (0.59 g, 1.75 mmol) and cesium carbonate (1.42 g, 4.37 mmol) at rt. The reaction mixture was degassed with N₂ gas for 5 min and then Pd(dppf)Cl₂·DCM (0.12 g, 0.14 mmol) was added. The reaction mixture was heated at 90° C. for 16 h and cooled to rt. The reaction mixture was filtered through a pad of celite and washed with EtOAc (10 mL). The filtrate was washed with brine, dried over anhydrous Na₂SO₄, filtered, and concentrated under reduced pressure to give crude residue. The crude was purified by silica gel column chromatography to obtain tert-butyl (3-(4-amino-7-methyl-7H-pyrrolo[2,3-d]pyrimidin-5-yl)-2-fluorophenyl)carbamate (0.36 g, 69%). LC-MS (ESI) m/z: 358.4 (M+H⁺).

A solution of tert-butyl (3-(4-amino-7-methyl-7H-pyrrolo[2,3-d]pyrimidin-5-yl)-2-fluorophenyl)carbamate (0.36 g, 1.0 mmol) in DCM (5 mL) was treated with 4.0 N HCl in 1,4-dioxane (1.0 mL, 4.0 mmol) at rt. The reaction mixture was stirred for 3 h and the solution was concentrated under reduced pressure to obtain 5-(3-amino-2-fluorophenyl)-7-methyl-7H-pyrrolo[2,3-d]pyrimidin-4-amine HCl salt (0.32 g, crude). LC-MS (ESI) m/z: 257.9 (M+H⁺).

### Suzuki Reaction

### Preparation of Example 1: 3-(N-(3-(8-amino-3-methylimidazo[1,5-a]pyrazin-1-yl)-2-fluorophenyl)sulfamoyl)-2,5-dichlorobenzyl acetate

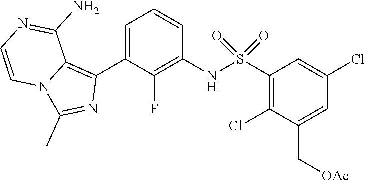 [SMILES: CC(=O)OCc1cc(Cl)cc(S(=O)(=O)Nc2cccc(-c3nc(C)n4ccnc(N)c34)c2F)c1Cl]

A solution of 1-iodo-3-methylimidazo[1,5-a]pyrazin-8-amine (L1, 3.0 g, 11 mmol) in a mixture of 1,4-dioxane:water (8:1, 67.5 mL) was treated with 2,5-dichloro-3-(N-(2-fluoro-3-(4,4,5,5-tetramethyl-1,3,2-dioxaborolan-2-yl)phenyl)sulfamoyl)benzyl acetate (G13, 6.8 g, 13 mmol) and K₂CO₃ (4.5 g, 33 mmol) at rt. The reaction mixture was degassed with Ar for 20 min and then Pd(dppf)Cl₂·DCM (0.89 g, 1.1 mmol) was added. The resulting mixture was heated at 90° C. for 12 h. The reaction mixture was cooled to rt and filtered through a pad of celite, washed thoroughly with EtOAc (500 mL). The filtrate was washed with sat'd NaHCO₃ (2×) and water. The organic layer was dried over anhydrous Na₂SO₄, filtered, and concentrated under reduced pressure. The crude was purified by reverse phase column chromatography (0 to 100% CH₃CN/water (0.1% FA)) to obtain 3-(N-(3-(8-amino-3-methylimidazo[1,5-a]pyrazin-1-yl)-2-fluorophenyl)sulfamoyl)-2,5-dichlorobenzyl acetate (5.9 g, 35%) as a light tan solid. ¹H NMR (400 MHz, DMSO-d₆): δ 10.76 (s, 1H), 7.88 (d, J=2.6 Hz, 1H), 7.78 (d, J=2.5 Hz, 1H), 7.43 (d, J=5.0 Hz, 1H), 7.22 (m, 2H), 7.16 (t, J=7.8 Hz, 1H), 6.98 (d, J=4.9 Hz, 1H), 5.84 (s, 2H), 5.13 (s, 2H), 2.48 (s, 3H), 2.02 (s, 3H).

### Hydrolysis

### Preparation of Example 2: N-(3-(8-amino-3-methylimidazo[1,5-a]pyrazin-1-yl)-2-fluorophenyl)-2,5-dichloro-3-(hydroxymethyl)benzenesulfonamide

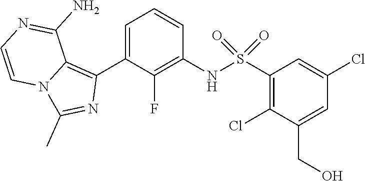 [SMILES: Cc1nc(-c2cccc(NS(=O)(=O)c3cc(Cl)cc(CO)c3Cl)c2F)c2c(N)nccn12]

A solution of 3-(N-(3-(8-amino-3-methylimidazo[1,5-a]pyrazin-1-yl)-2-fluorophenyl)sulfamoyl)-2,5-dichlorobenzyl acetate (1, 0.53 g, 0.98 mmol) in MeOH (5 mL) was treated with K₂CO₃ (0.40 g, 2.9 mmol). The suspension was stirred at rt for 1 h and the reaction mixture was directly concentrated under reduced pressure. The crude was acidified with 10% citric acid solution (~50 mL, pH~5,) and then the solid was start precipitated out. The solid was filtered, washed with water, and dried under high vacuum to obtain N-(3-(8-amino-3-methylimidazo[1,5-a]pyrazin-1-yl)-2-fluorophenyl)-2,5-dichloro-3-(hydroxymethyl)benzenesulfonamide (0.12 g, 25%). ¹H NMR (400 MHz, DMSO-d₆): δ 10.77 (br m, 1H), 7.85 (d, J=2.4 Hz, 1H), 7.75 (br s, 1H), 7.50 (d, J=4.8 Hz, 1H), 7.27 (m, 1H), 7.20 (m, 2H), 7.04 (d, J=4.8 Hz, 1H), 5.84 (br s, 2H), 5.70 (t, J=6.0 Hz, 1H), 4.60 (d, J=5.6, 2H), 2.54 (s, 3H); MS (ESI) m/z: 496.3 (M+H⁺).

The following compounds are prepared essentially by method of preparation of examples 1 and 2.

| Ex | | Yield | ¹H NMR | MS (m/z: |
|---|---|---|---|---|
| No | Product | (%) | (400 MHz, DMSO-d₆): δ | M + H⁺) |
| 3 | 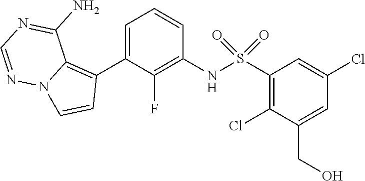 [SMILES: Nc1ncnn2ccc(-c3cccc(NS(=O)(=O)c4cc(Cl)cc(CO)c4Cl)c3F)c12] | 25 | 10.69 (s, 1H), 7.87 (s, 1H), 7.84 (d, J = 2.6 Hz, 1H), 7.77 (m, 1H), 7.74 (d, J = 2.7 Hz, 1H), 7.24 (m, 3H), 6.59 (dd, J = 0.9, 2.8 Hz, 1H), 5.71 (t, J = 5.7 Hz, 1H), 4.59 (d, J = 5.6 Hz, 2H). NH₂ protons are missing. | 482.0 |
| 4 | 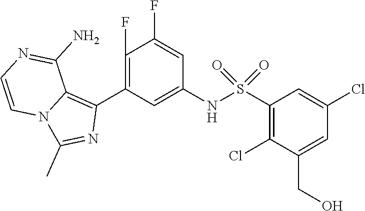 [SMILES: Cc1nc(-c2cc(NS(=O)(=O)c3cc(Cl)cc(CO)c3Cl)cc(F)c2F)c2c(N)nccn12] | 43 | 11.13 (s, 1H), 7.97 (d, J = 2.6 Hz, 1H), 7.79 (d, J = 2.6 Hz, 1H), 7.49 (d, J = 5.2 Hz, 1H), 7.15 (m, 1H), 7.07 (d, J = 4.8 Hz, 1H), 6.97 (d, J = 4.9 Hz, 1H), 6.07 (s, 2H), 5.70 (s, 1H), 4.59 (d, J = 4.1 Hz, 2H), 2.55 (s, 3H). | 513.0 |
| 5 | 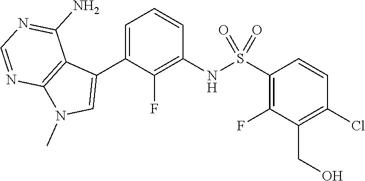 [SMILES: Cn1cc(-c2cccc(NS(=O)(=O)c3ccc(Cl)c(CO)c3F)c2F)c2c(N)ncnc21] | 23 | 10.68 (s, 1H), 8.15 (s, 1H), 7.72 (t, J = 8.0 Hz, 1H), 7.48 (d, J = 8.8 Hz, 1H), 7.26 (s, 1H), 7.21 (br m, 3H), 5.99 (br s, 2H), 5.48 (t, J = 5.2 Hz, 1H), 4.61 (d, J = 4.0 Hz, 2H), 3.73 (s, 3H). | 480.2 |
| 6 | 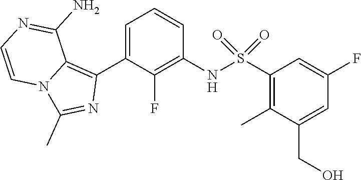 [SMILES: Cc1c(CO)cc(F)cc1S(=O)(=O)Nc1cccc(-c2nc(C)n3ccnc(N)c23)c1F] | 23 | 10.54 (br s, 1H), 7.45-7.57 (m, 3H), 7.18-7.33 (m, 3H), 7.04 (d, J = 4.8 Hz, 1H), 5.81 (br s, 2H), 5.47 (t, J = 5.2 Hz, 1H), 4.54 (d, J = 5.2 Hz, 2H), 2.55 (s, 3H), 2.45 (s, 3H). | 460.4 |
| 7 | 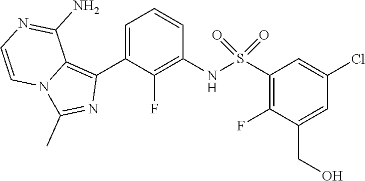 [SMILES: Cc1nc(-c2cccc(NS(=O)(=O)c3cc(Cl)cc(CO)c3F)c2F)c2c(N)nccn12] | 7 | 10.75 (br s, 1H), 7.70 (br m, 1H), 7.64 (m, 1H), 7.49 (d, J = 5.2 Hz, 1H), 7.28 (br m, 1H), 7.20 (br m, 2H), 7.04 (d, J = 4.8 Hz, 1H), 5.83 (br s, 2H), 5.55 (br t, J = 6.0 Hz, 1H), 4.55 (d, J = 6.0 Hz, 2H), 2.55 (s, 3H). | 479.9 |
| 8 | 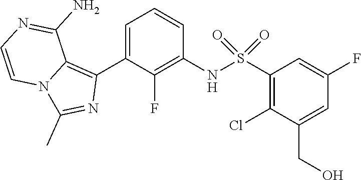 [SMILES: Cc1nc(-c2cccc(NS(=O)(=O)c3cc(F)cc(CO)c3Cl)c2F)c2c(N)nccn12] | 27 | 10.70 (br s, 1H), 7.67 (dd, J = 2.8, 8.0 Hz, 1H), 7.53 (br d, J = 7.6 Hz, 1H), 7.48 (d, J = 4.8 Hz, 1H), 7.26 (br m, 1H), 7.15 (br m, 2H), 7.03 (d, J = 4.8 Hz, 1H), 5.82 (br s, 2H), 5.68 (br t, J = 5.6 Hz, 1H), 4.59 (d, J = 5.6 Hz, 2H), 2.55 (s, 3H). | 480.4 |
| 9 | 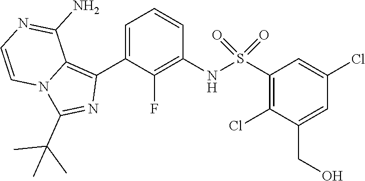 [SMILES: CC(C)(C)c1nc(-c2cccc(NS(=O)(=O)c3cc(Cl)cc(CO)c3Cl)c2F)c2c(N)nccn12] | 33 | 10.91 (br s, 1H), 7.87 (d, J = 2.6 Hz, 1H), 7.83 (d, J = 5.4 Hz, 1H), 7.79 (d, J = 2.6 Hz, 1H), 7.29-7.37 (m, 2H), 7.25 (t, J = 7.8 Hz, 1H), 7.00 (d, J = 5.4 Hz, 1H), 6.53 (br s, 2H), 5.74 (t, J = 5.7 Hz, 1H), 4.60 (d, J = 4.4 Hz, 2H), 1.46 (s, 9H). | 528.0 |
| 10 | 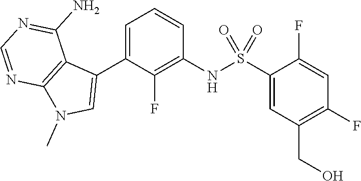 [SMILES: Cn1cc(-c2cccc(NS(=O)(=O)c3cc(CO)c(F)cc3F)c2F)c2c(N)ncnc21] | 17 | 10.6 (br s, 1H), 8.15 (s, 1H), 7.89 (t, J = 8.0 Hz, 1H), 7.52 (t, J = 10.0 Hz, 1H), 7.26 (s, 1H), 7.22 (m, 3H), 5.94 (br s, 2H), 5.52 (t, J = 6.0 Hz, 1H), 4.52 (d, J = 5.6 Hz, 2H), 3.73 (s, 3H). | 464.3 |
| 11 | 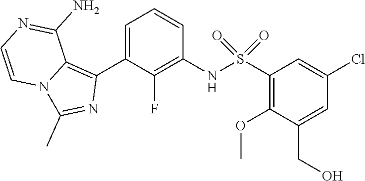 [SMILES: COc1c(CO)cc(Cl)cc1S(=O)(=O)Nc1cccc(-c2nc(C)n3ccnc(N)c23)c1F] | 67 | 10.29 (br s, 1H), 7.68 (br m, 1H), 7.63 (d, J = 2.8 Hz, 1H), 7.49 (d, J = 4.8 Hz, 1H), 7.29 (m, 1H), 7.14-7.26 (br m, 2H), 7.04 (d, J = 5.2 Hz, 1H), 5.83 (br s, 2H), 5.50 (t, J = 6.0 Hz, 1H), 4.56 (d, J = 5.6 Hz, 2H), 3.77 (s, 3H), 2.51 (s, 3H). | 492.0 |
| 12 | 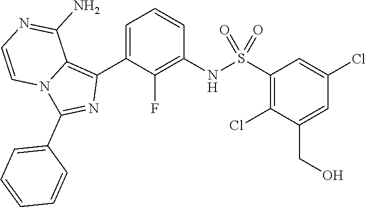 [SMILES: Nc1nccn2c(-c3ccccc3)nc(-c3cccc(NS(=O)(=O)c4cc(Cl)cc(CO)c4Cl)c3F)c12] | 9 | 10.81 (br s, 1H), 7.88 (d, J = 2.4 Hz, 1H), 7.78- 7.84 (m, 3H), 7.55-7.61 (m, 3H), 7.35-7.42 (m, 2H ), 7.29 (t, J = 7.6 Hz, 1H), 7.10 (d, J = 5.2 Hz, 1H), 6.69 (br s, 2H), 5.72 (br s, 1H), 4.60 (s 2H). | 558.3 |
| 13 | 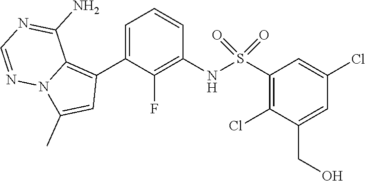 [SMILES: Cc1cc(-c2cccc(NS(=O)(=O)c3cc(Cl)cc(CO)c3Cl)c2F)c2c(N)ncnn12] | 1 | 10.69 (s, 1H), 7.91 (s, 1H), 7.84 (d, J = 2.8 Hz, 1H), 7.76 (br s, 1H), 7.52-7.64 (m, 2H), 7.17- 7.21 (br m, 3H), 6.45 (s, 1H), 5.71 (t, J = 5.2 Hz, 1H), 4.60 (d, J = 5.6 Hz, 2H), 2.49 (s, 3H). | 496.2 |
| 14 | 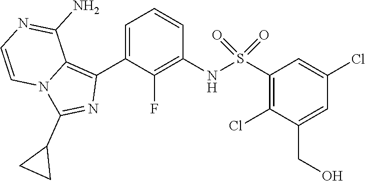 [SMILES: Nc1nccn2c(C3CC3)nc(-c3cccc(NS(=O)(=O)c4cc(Cl)cc(CO)c4Cl)c3F)c12] | 25 | 10.72 (br s, 1H), 7.85 (d, J = 2.8 Hz, 1H), 7.77 (d, J = 2.8 Hz, 1H), 7.72 (d, J = 5.2 Hz, 1H), 7.19-7.30 (m, 3H), 7.06 (d, J = 4.8 Hz, 1H ), 5.86 (br s, 2H), 5.71 (t, J = 5.6 Hz, 1H), 4.60 (d, J = 6.0 Hz, 2H), 2.31 (m, 1H), 1.03 (m, 2H), 0.93 (m, 2H). | 522.2 |
| 15 | 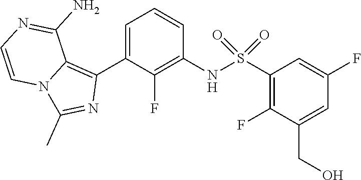 [SMILES: Cc1nc(-c2cccc(NS(=O)(=O)c3cc(F)cc(CO)c3F)c2F)c2c(N)nccn12] | 19 | 10.75 (br s, 1H), 7.55 (m, 1H), 7.49 (d, J = 6.0 Hz, 1H), 7.45 (m, 1H), 7.27-7.35 (m, 2H), 7.23 (m, 1H), 7.05 (d, J = 5.2 Hz, 1H), 5.86 (br s, 2H), 5.57 (t, J = 6.0 Hz, 1H), 4.57 (d, J = 5.6 Hz, 2H), 2.55 (s, 3H). | 464.4 |
| 16 | 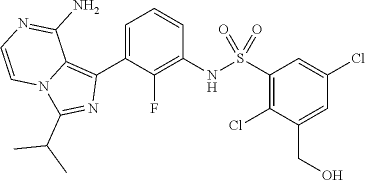 [SMILES: CC(C)c1nc(-c2cccc(NS(=O)(=O)c3cc(Cl)cc(CO)c3Cl)c2F)c2c(N)nccn12] | 14 | 10.77 (br s, 1H), 7.89 (d, J = 2.8 Hz, 1H), 7.84 (d, J = 5.6 Hz, 1H), 7.80 (d, J = 2.8 Hz, 1H), 7.32-7.39 (m, 2H), 7.27 (m, 1H), 7.07 (d, J = 5.6 Hz, 1H), 5.73 (br s, 1H), 4.61 (s, 2H), 3.50 (m, 1H), 1.31 (d, J = 6.8 Hz, 6H). NH₂ protons are missing. | 524.3 |
| 17 | 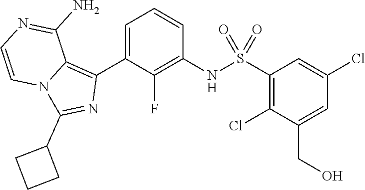 [SMILES: Nc1nccn2c(C3CCC3)nc(-c3cccc(NS(=O)(=O)c4cc(Cl)cc(CO)c4Cl)c3F)c12] | 15 | 10.71 (br s, 1H), 7.86 (d, J = 2.8 Hz, 1H), 7.77 (d, J = 2.4 Hz, 1H), 7.44 (d, J = 4.8 Hz, 1H), 7.26-7.35 (m, 2H), 7.23 (m, 1H), 7.01 (d, J = 5.2 Hz, 1H), 5.85 (br s, 2H), 5.70 (t, J = 6.0 Hz, 1H), 4.60 (d, J = 5.6 Hz, 2H), 3.93 (m, 1H), 2.34-2.40 (m, 4H), 2.17 (m, 1H), 1.90 (m, 1H). | 536.3 |
| 18 | 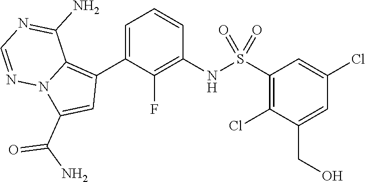 [SMILES: NC(=O)c1cc(-c2cccc(NS(=O)(=O)c3cc(Cl)cc(CO)c3Cl)c2F)c2c(N)ncnn12] | 19 | 10.81 (s, 1H), 8.62 (d, J = 5.2 Hz, 1H), 8.01 (br s, 1H), 7.86 (d, J = 2.4 Hz, 1H), 7.78 (d, J = 2.8 Hz, 1H), 7.70 (br s, 1H), 7.37 (br t, J = 7.2 Hz, 2H), 7.25-7.29 (m, 2H), 6.05 (br s, 2H), 5.73 (t, J = 5.8 Hz, 1H), 4.60 (d, J = 5.6 Hz, 2H). | 525.1 |
| 19 | 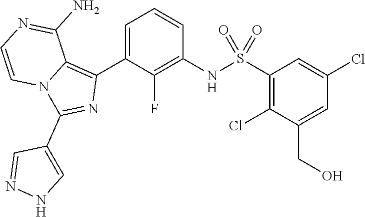 [SMILES: Nc1nccn2c(-c3cn[nH]c3)nc(-c3cccc(NS(=O)(=O)c4cc(Cl)cc(CO)c4Cl)c3F)c12] | 1 | 13.34 (br s, 1H), 10.77 (br s, 1H), 8.46 (br s, 1H), 8.06 (br s, 1H), 7 .86 (d, J = 2.4 Hz, 1H), 7.76 (s, 1H), 7.75 (s, 1H), 7.23-7.34 (m, 3H), 7.11 (d, J = 5.2 Hz, 1H), 5.91 (br s, 2H), 5.70 (t, J = 5.8 Hz, 1H), 4.60 (d, J = 5.6 Hz, 2H). | 548.1 |
| 20 | 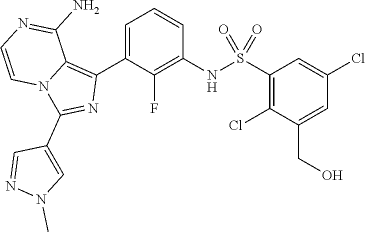 [SMILES: Cn1cc(-c2nc(-c3cccc(NS(=O)(=O)c4cc(Cl)cc(CO)c4Cl)c3F)c3c(N)nccn23)cn1] | 5 | 10.76 (br s, 1H), 8.43 (s, 1H), 8.00 (s, 1H), 7.86 (d, J = 2.4 Hz, 1H), 7.77 (s, 1H), 7.72 (d, J = 4.8 Hz, 1H), 7.31-7.34 (m, 2H), 7.24 (m, 1H), 7.12 (d, J = 4.8 Hz, 1H), 5.92 (br s, 2H), 5.70 (t, J = 6.0 Hz, 1H), 4.60 (d, J = 5.6 Hz, 2H), 3.93 (s, 3H). | 562.2 |
| 21 | 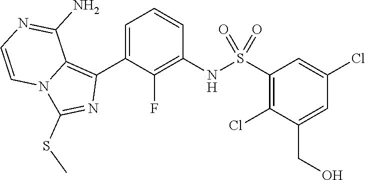 [SMILES: CSc1nc(-c2cccc(NS(=O)(=O)c3cc(Cl)cc(CO)c3Cl)c2F)c2c(N)nccn12] | 14 | 10.76 (br s, 1H), 7.85 (d, J = 2.8 Hz, 1H), 7.78 (d, J = 2.8 Hz, 1H), 7.49 (d, J = 4.8 Hz, 1H), 7.33 (m, 2H), 7.25 (m, 1H), 7.14 (d, J = 4.8 Hz, 1H), 6.00 (br s, 2H), 5.71 (t, J = 6.0 Hz, 1H), 4.60 (d, J = 5.6 Hz, 2H), 2.58 (s, 3H). | 528.1 |
| 22 | 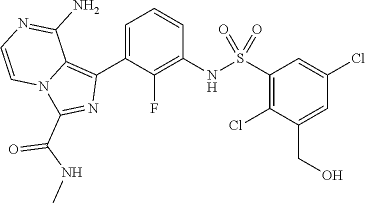 [SMILES: CNC(=O)c1nc(-c2cccc(NS(=O)(=O)c3cc(Cl)cc(CO)c3Cl)c2F)c2c(N)nccn12] | 30 | 10.82 (br s, 1H), 8.63 (m, 2H), 7.85 (d, J = 2.4 Hz, 1H), 7.74 (br s, 1H), 7.33 (m, 2H), 7.27 (d, J = 4.8 Hz, 1H), 7.22 (br s, 1H), 6.04 (br s, 2H), 5.17 (br m, 1H), 4.56 (d, J = 5.6 Hz, 2H), 2.79 (d, J = 4.4 Hz, 3H). | 539.5 |
| 23 | 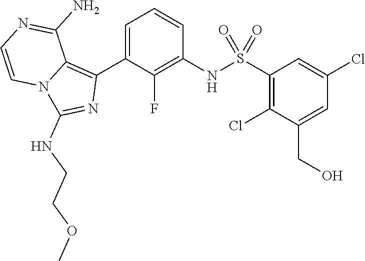 [SMILES: COCCNc1nc(-c2cccc(NS(=O)(=O)c3cc(Cl)cc(CO)c3Cl)c2F)c2c(N)nccn12] | 47 | 10.75 (br s, 1H), 7.83 (d, J = 2.8 Hz, 1H), 7.73 (d, J = 2.0 Hz, 1H), 7.32 (d, J = 5.2 Hz, 1H), 7.24 (m, 1H), 7.15 (m, 2H), 6.77 (d, J = 5.2 Hz, 1H), 6.69 (br t, J = 4.8 Hz, 1H), 5.82 (br s, 2H), 5.69 (t, J = 5.6 Hz, 1H), 4.58 (d, J = 5.2 Hz, 2H), 3.47-3.51 (m, 4H), 3.26 (s, 3H). | 555.2 |
| 24 | 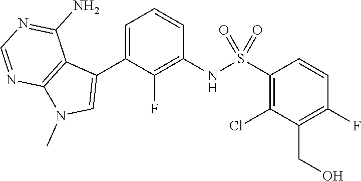 [SMILES: Cn1cc(-c2cccc(NS(=O)(=O)c3ccc(F)c(CO)c3Cl)c2F)c2c(N)ncnc21] | 14 | 10.57 (s, 1H), 8.14 (s, 1H), 7.99 (dd, J = 6.0, 8.8 Hz, 1H), 7.38 (t, J = 8.8 Hz, 1H), 7.27 (s, 1H), 7.17 (br m, 3H), 5.97 (br s, 2H), 5.41 (t, J = 4.8 Hz, 1H), 4.64 (d, J = 4.0 Hz, 2H), 3.73 (s, 3H). | 480.3 |
| 25 | 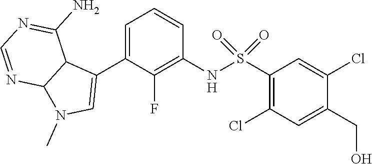 [SMILES: CN1C=C(c2cccc(NS(=O)(=O)c3cc(Cl)c(CO)cc3Cl)c2F)C2C(N)=NC=NC21] | 2.5 | 10.68 (s, 1H), 8.15 (s, 1H), 7.89 (s, 1H), 7.74 (d, J = 1.1 Hz, 1H), 7.26 (s, 1H), 7.19-7.23 (m, 3H), 6.00 (br s, 2H), 5.74 (t, J = 5.6 Hz, 1H), 4.57 (d, J = 5.7 Hz, 1H), 3.73 (s, 3H), OH is missing. | 496.0 |
| 26 | 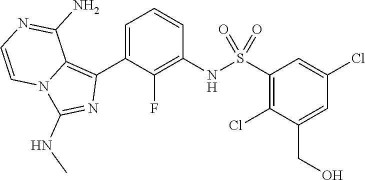 [SMILES: CNc1nc(-c2cccc(NS(=O)(=O)c3cc(Cl)cc(CO)c3Cl)c2F)c2c(N)nccn12] | 15 | 10.74 (br s, 1H), 7.84 (d, J = 2.4 Hz, 1H), 7.74 (s, 1H), 7.14-7.26 (m, 4H), 6.77 (d, J = 5.2 Hz, 1H), 6.51 (br q, J = 4.4 Hz, 1H), 5.82 (br s, 2H), 5.68 (t, J = 5.8 Hz, 1H), 4.58 (d, J = 5.2 Hz, 2H), 2.88 (d, J = 4.4 Hz, 3H). | 511.2 |
| 27 | 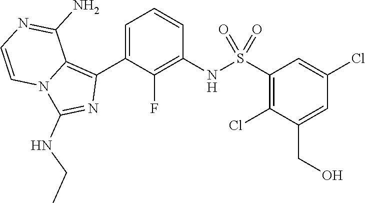 [SMILES: CCNc1nc(-c2cccc(NS(=O)(=O)c3cc(Cl)cc(CO)c3Cl)c2F)c2c(N)nccn12] | 34 | 10.72 (br s, 1H), 7.83 (d, J = 2.8 Hz, 1H), 7.74 (d, J = 2.0 Hz, 1H), 7.21-7.26 (m, 2H), 7.15- 7.17 (m, 2H), 6.77 (d, J = 4.8 Hz, 1H), 6.52 (br t, J = 5.2 Hz, 1H), 5.82 (br s, 2H), 5.69 (t, J = 5.8 Hz, 1H), 4.54 (d, J = 5.2 Hz, 2H), 3.35 (m, 2H), 1.18 (t, J = 4.4 Hz, 3H). | 525.2 |

The following compounds are prepared essentially by method of preparation of example 1

| Ex | | Yield | ¹H NMR | MS (m/z: |
|---|---|---|---|---|
| No | Product | (%) | (400 MHz, DMSO-d₆): δ | M + H⁺) |
| 28 | 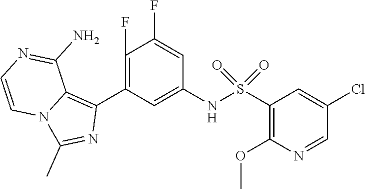 [SMILES: COc1ncc(Cl)cc1S(=O)(=O)Nc1cc(F)c(F)c(-c2nc(C)n3ccnc(N)c23)c1] | 51 | 10.79 (s, 1H), 8.49 (d, J = 2.6 Hz, 1H), 8.24 (d, J = 2.5 Hz, 1H), 7.49 (d, J = 5.0 Hz, 1H), 7.15 (m, 1H), 7.07 (d, J = 4.8 Hz, 1H), 7.00 (m, 1H), 6.04 (s, 2H), 3.96 (s, 3H), 2.55 (s, 3H). | 481.2 |
| 29 | 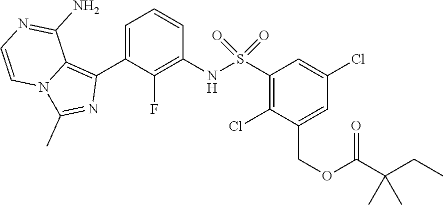 [SMILES: CCC(C)(C)C(=O)OCc1cc(Cl)cc(S(=O)(=O)Nc2cccc(-c3nc(C)n4ccnc(N)c34)c2F)c1Cl] | 34 | 10.78 (br s, 1H), 7.93 (d, J = 2.8 Hz, 1H), 7.82 (d, J = 2.4 Hz, 1H), 7.50 (d, J = 4.8 Hz, 1H), 7.20-7.32 (m, 3H), 7.04 (d, J = 4.8 Hz, 1H), 5.88 (br s, 2H), 5.18 (s, 2H), 2.54 (s, 3H), 1.43 (q, J = 7.2 Hz, 2H), 1.04 (s, 6H), 0.70 (t, J = 7.6 Hz, 3H). | 594.2 |
| 30 | 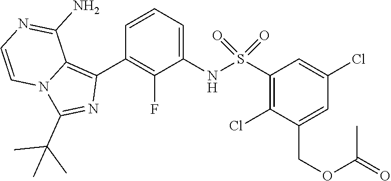 [SMILES: CC(=O)OCc1cc(Cl)cc(S(=O)(=O)Nc2cccc(-c3nc(C(C)(C)C)n4ccnc(N)c34)c2F)c1Cl] | 84 | 10.75 (s, 1H), 7.94 (d, J = 2.6 Hz, 1H), 7.72 (d, J = 5.1 Hz, 1H), 7.67 (s, 1H), 7.21 (t, J = 8.0 Hz, 1H), 7.01 (s, 1H), 6.96 (d, J = 5.1 Hz, 1H), 6.88 (s, 1H), 5.78-5.82 (m, 2H), 5.16 (s, 2H), 2.09 (s, 3H), 1.46 (s, 9H). | 580.1 |
| 31 | 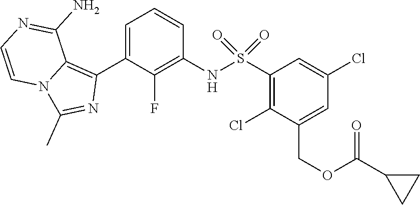 [SMILES: Cc1nc(-c2cccc(NS(=O)(=O)c3cc(Cl)cc(COC(=O)C4CC4)c3Cl)c2F)c2c(N)nccn12] | 9 | 10.73 (s, 1H), 7.88 (d, J = 2.5 Hz, 1H), 7.75 (d, J = 2.6 Hz, 1H), 7.42 (d, J = 4.9 Hz, 2H), 7.18- 7.25 (m, 2H), 7.13 (q, J = 6.8 Hz, 2H), 6.97 (d, J = 5.0 Hz, 1H), 5.79 (s, 1H), 5.13 (s, 2H), 2.48 (s, 3H), 1.65 (m, 2H), 0.77-0.86 (m, 4H). | 564.2 |
| 32 | 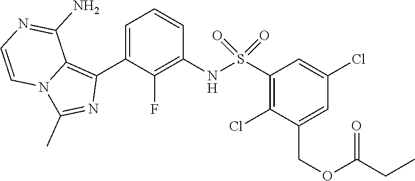 [SMILES: CCC(=O)OCc1cc(Cl)cc(S(=O)(=O)Nc2cccc(-c3nc(C)n4ccnc(N)c34)c2F)c1Cl] | 30 | 10.80 (br s, 1H), 7.94 (d, J = 2.4 Hz, 1H), 7.84 (d, J = 2.4 Hz, 1H), 7.50 (d, J = 4.8 Hz, 1H), 7.20-7.32 (m, 3H), 7.05 (d, J = 5.2 Hz, 1H), 5.90 (br s, 2H), 5.20 (s, 2H), 2.55 (s, 3H), 2.39 (q, J = 7.6 Hz, 2H), 1.01 (t, J = 8.0 Hz, 3H). | 552.1 |
| 33 | 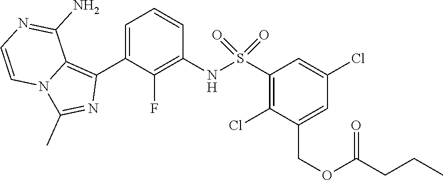 [SMILES: CCCC(=O)OCc1cc(Cl)cc(S(=O)(=O)Nc2cccc(-c3nc(C)n4ccnc(N)c34)c2F)c1Cl] | 9 | 10.81 (br s, 1H), 7.94 (d, J = 2.4 Hz, 1H), 7.78 (br s, 1H), 7.49 (d, J = 5.6 Hz, 1H), 7.27 (m, 1H), 7.25 (br s, 2H), 7.03 (d, J = 5.2 Hz, 1H), 5.84 (br s, 2H), 5.19 (s, 2H), 2.54 (s, 3H), 2.35 (t, J= 7.2 Hz, 2H), 1.52 (m, 2H), 0.85 (t, J = 7.3 Hz, 3H). | 566.2 |
| 34 | 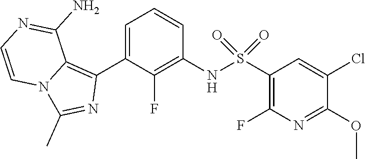 [SMILES: COc1nc(F)c(S(=O)(=O)Nc2cccc(-c3nc(C)n4ccnc(N)c34)c2F)cc1Cl] | 37 | 10.56 (s, 1H), 7.73 (d, J = 7.3 Hz, 1H), 7.49 (d, J = 5.0 Hz, 1H), 7.31-7.39 (m, 2H), 7.21-7.30 (m, 2H), 7.05 (d, J = 5.0 Hz, 1H), 5.85 (s, 2H), 3.93 (s, 3H), 2.55 (s, 3H). | 480.0 |
| 35 | 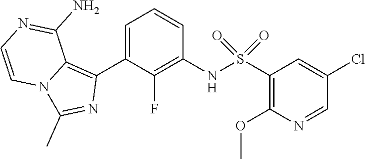 [SMILES: COc1ncc(Cl)cc1S(=O)(=O)Nc1cccc(-c2nc(C)n3ccnc(N)c23)c1F] | 51 | 10.43 (br s, 1H), 8.46 (d, J = 2.6 Hz, 1H), 8.08 (d, J = 2.6 Hz, 1H), 7.48 (d, J = 5.0 Hz, 1H), 7.33 (dt, J = 1.6, 7.2 Hz, 1H), 7.20-7.31 (m, 2H), 7.03 (d, J = 5.0 Hz, 1H), 5.83 (s, 2H), 3.89 (s, 3H), 2.53 (s, 3H). | 463.0 |
| 35 | 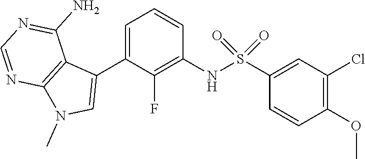 [SMILES: COc1ccc(S(=O)(=O)Nc2cccc(-c3cn(C)c4ncnc(N)c34)c2F)cc1Cl] | 23 | 10.21 (br s, 1H), 8.15 (s, 1H), 7.77 (d, J = 1.6 Hz, 1H), 7.69 (br d, J = 8.8 Hz, 1H), 7.31 (d, J = 8.8 Hz, 1H), 7.17-7.24 (m, 4H), 6.00 (br s, 2H), 3.92 (s, 3H), 3.73 (s, 3H). | 462.2 |
| 36 | 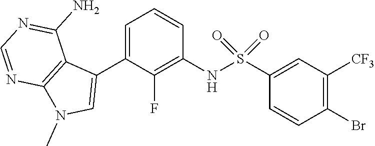 [SMILES: Cn1cc(-c2cccc(NS(=O)(=O)c3ccc(Br)c(C(F)(F)F)c3)c2F)c2c(N)ncnc21] | 37 | 10.53 (br s, 1H), 8.04-8.20 (m, 3H), 7.90 (d, J = 8.0 Hz, 1H), 7.18 (m, 4H), 5.99 (br s, 2H), 3.73 (s, 3H). | 544.0 546.0 |
| 37 | 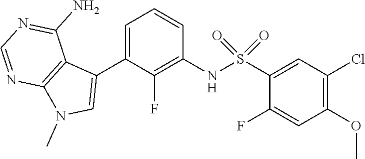 [SMILES: COc1cc(F)c(S(=O)(=O)Nc2cccc(-c3cn(C)c4ncnc(N)c34)c2F)cc1Cl] | 36 | 10.53 (br s, 1H), 8.15 (s, 1H), 7.71 (d, J = 7.3 Hz, 1H), 7.36 (d, J = 11.9 Hz, 1H), 7.25 (s, 1H), 7.19-7.24 (m, 3H), 6.00 (br s, 2H), 3.94 (s, 3H), 3.73 (s, 3H). | 480.0 |
| 38 | 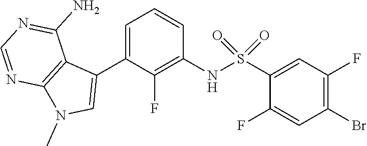 [SMILES: Cn1cc(-c2cccc(NS(=O)(=O)c3cc(F)c(Br)cc3F)c2F)c2c(N)ncnc21] | 20 | 10.82 (br s, 1H), 8.15 (s, 1H), 8.09 (dd, J = 5.2, 8.8 Hz, 1H), 7.73 (dd, J = 6.0, 7.6 Hz, 1H), 7.20-7.30 (m, 4H), 6.02 (br s, 2H), 3.73 (s, 3H). | 514.0 516.0 |
| 39 | 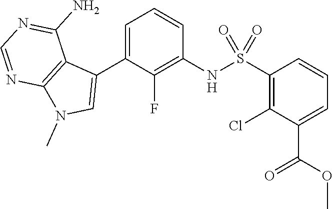 [SMILES: COC(=O)c1cccc(S(=O)(=O)Nc2cccc(-c3cn(C)c4ncnc(N)c34)c2F)c1Cl] | 23 | 10.71 (s, 1H), 8.15 (s, 1H), 8.14 (dd, J = 1.7, 8.0 Hz, 1H), 7.96 (dd, J = 1.7, 7.7 Hz, 1H), 7.61 (t, J = 7.9 Hz, 1H), 7.26 (s, 1H), 7.15-7.23 (m, 3H), 6.01 (br s, 2H), 3.89 (s, 3H), 3.73 (s, 3H). | 490.0 |
| 40 | 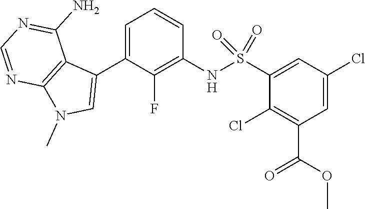 [SMILES: COC(=O)c1cc(Cl)cc(S(=O)(=O)Nc2cccc(-c3cn(C)c4ncnc(N)c34)c2F)c1Cl] | 20 | 10.86 (br s, 1H), 8.15 (s, 1H), 8.11 (d, J = 2.8 Hz, 1H), 8.09 (d, J = 2.4 Hz, 1H), 7.27 (s, 1H), 7.22 (m, 3H), 6.03 (br s, 2H), 3.89 (s, 3H), 3.73 (s, 3H). | 524.2 |
| 41 | 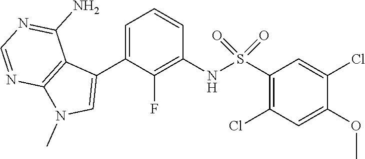 [SMILES: COc1cc(Cl)c(S(=O)(=O)Nc2cccc(-c3cn(C)c4ncnc(N)c34)c2F)cc1Cl] | 25 | 10.52 (s, 1H), 8.15 (s, 1H), 7.90 (s, 1H), 7.46 (s, 1H), 7.26 (s, 1H), 7.15-7.24 (m, 3H), 6.01 (s, 2H), 3.96 (s, 3H), 3.73 (s, 3H). | 496.0 |
| 42 | 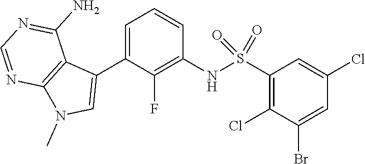 [SMILES: Cn1cc(-c2cccc(NS(=O)(=O)c3cc(Cl)cc(Br)c3Cl)c2F)c2c(N)ncnc21] | 20 | 10.88 (br s, 1H), 8.29 (d, J = 2.0 Hz, 1H), 8.15 (s, 1H), 7.95 (d, J = 2.4 Hz, 1H), 7.28 (s, 1H), 7.18-7.24 (m, 3H), 6.04 (br s, 2H), 3.73 (s, 3H). | 544.2 |
| 43 | 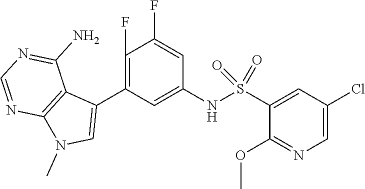 [SMILES: COc1ncc(Cl)cc1S(=O)(=O)Nc1cc(F)c(F)c(-c2cn(C)c3ncnc(N)c23)c1] | 52 | 10.75 (s, 1H), 8.47 (d, J = 2.5 Hz, 1H), 8.24 (d, J = 2.5 Hz, 1H), 8.14 (s, 1H), 7.28 (s, 1H), 7.03 (m, 1H), 6.90 (d, J = 5.3 Hz, 1H), 6.17 (s, 2H),3.94 (s, 3H), 3.72 (s, 3H). | 481.2 |
| 44 | 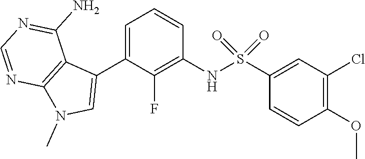 [SMILES: COc1ccc(S(=O)(=O)Nc2cccc(-c3cn(C)c4ncnc(N)c34)c2F)cc1Cl] | 23 | 10.21 (br s, 1H), 8.15 (s, 1H), 7.77 (d, J = 1.6 Hz, 1H), 7.69 (br d, J = 8.8 Hz, 1H), 7.31 (d, J = 8.8 Hz, 1H), 7.17-7.24 (m, 4H), 6.00 (br s, 2H), 3.92 (s, 3H), 3.73 (s, 3H). | 462.2 |

### Formation of Sulfonamide

### Preparation of Example 45: N-(3-(4-amino-7-methyl-7H-pyrrolo[2,3-d]pyrimidin-5-yl)-2-fluorophenyl)-5-chloro-2-methoxypyridine-3-sulfonamide

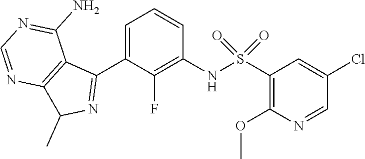 [SMILES: COc1ncc(Cl)cc1S(=O)(=O)Nc1cccc(C2=NC(C)c3ncnc(N)c32)c1F]

A solution of 5-(3-amino-2-fluorophenyl)-7-methyl-7H-pyrrolo[2,3-d]pyrimidin-4-amine HCl salt (N1, 0.06 g, 0.20 mmol) in pyridine (3 mL) was treated with 5-chloro-2-methoxypyridine-3-sulfonyl chloride (E12, 0.06 g, 0.24 mmol) at rt. The reaction mixture was stirred at rt for 16 h and then diluted with EtOAc (20 mL). The solution was washed with sat'd NaHCO₃ and then the organic layer was dried over anhydrous Na2SO4, filtered, and concentrated under reduced pressure to obtain the crude material. The crude material was further triturated with Et₂O and the solid was filtered to give the pure N-(3-(4-amino-7-methyl-7H-pyrrolo[2,3-d]pyrimidin-5-yl)-2-fluorophenyl)-5-chloro-2-methoxypyridine-3-sulfonamide (0.025 g, 27%) as an off white solid. ¹H NMR (400 MHz, DMSO-d₆): δ 10.39 (br s, 1H), 8.49 (d, J=2.4 Hz, 1H), 8.15 (s, 1H), 8.10 (d, J=2.4 Hz, 1H), 7.26 (s, 1H), 7.22 (m, 3H), 5.99 (br s, 2H), 3.91 (s, 3H), 3.73 (s, 3H); LC-MS (ESI) m/z: 463.2 (M+H⁺).

### Nucleophilic Substitution Reaction

### Preparation of Example 46: N-(3-(8-amino-3-methylimidazo[1,5-a]pyrazin-1-yl)-2-fluorophenyl)-2,5-dichloro-3-(chloromethyl)benzenesulfonamide

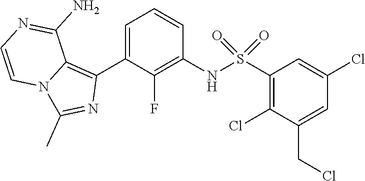 [SMILES: Cc1nc(-c2cccc(NS(=O)(=O)c3cc(Cl)cc(CCl)c3Cl)c2F)c2c(N)nccn12]

A solution of N-(3-(8-amino-3-methylimidazo[1,5-a]pyrazin-1-yl)-2-fluorophenyl)-2,5-dichloro-3-(hydroxymethyl)benzenesulfonamide (2, 0.20 g, 0.404 mmol) in 1,2-dichloroethane (7 mL) under nitrogen atmosphere was treated with POCl₃ (0.11 mL, 1.21 mmol) at rt. The reaction mixture was stirred at rt for 6 h and then the mixture was quenched with ice-cold water (20 mL) slowly. The aqueous layer was neutralized with sat'd NaHCO₃ solution and then the aqueous layer was extracted with 10% MeOH/DCM (3×). The combined organics were dried over anhydrous Na₂SO₄, filtered, and concentrated under reduced pressure to give of N-(3-(8-amino-3-methylimidazo[1,5-a]pyrazin-1-yl)-2-fluorophenyl)-2,5-dichloro-3-(chloromethyl)benzenesulfonamide (0.2 g, 62%) as a light brown solid. MS (ESI) m/z: 514.0 (M+H⁺).

The following compounds are prepared essentially by method of preparation of example 46.

| Ex | | Yield | ¹H NMR | MS (m/z: |
|---|---|---|---|---|
| No | Product | (%) | (400 MHz, DMSO-d₆): δ | M + H⁺) |
| 47 | 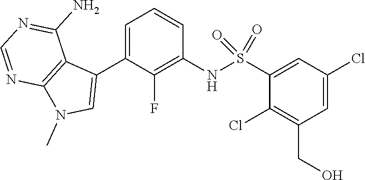 [SMILES: Cn1cc(-c2cccc(NS(=O)(=O)c3cc(Cl)cc(CO)c3Cl)c2F)c2c(N)ncnc21] | 15 | 8.15 (s, 1H), 7.91 (d, J = 2.4 Hz, 1H), 7.81 (d, J | 495.3 |

### Nucleophilic Substitution Reaction

### Preparation of Example 48: N-(3-(8-amino-3-methylimidazo[1,5-a]pyrazin-1-yl)-2-fluorophenyl)-2,5-dichloro-3-(methoxymethyl)benzenesulfonamide

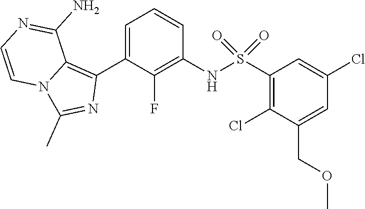 [SMILES: COCc1cc(Cl)cc(S(=O)(=O)Nc2cccc(-c3nc(C)n4ccnc(N)c34)c2F)c1Cl]

A solution of N-(3-(8-amino-3-methylimidazo[1,5-a]pyrazin-1-yl)-2-fluorophenyl)-2,5-dichloro-3-(chloromethyl)benzenesulfonamide (46, 0.20 g, 0.39 mmol) in MeOH (5 mL) was treated with 25% NaOMe in MeOH (5 mL) at rt. The reaction mixture was stirred at rt for 16 h and then the mixture was quenched with ice-cold water (20 mL) slowly. The aqueous layer was extracted with 10% MeOH/DCM (3×) and the combined organics were dried over anhydrous Ns₂SO₄, filtered, and concentrated under reduced pressure. The crude material was purified by silica gel column chromatography (0 to 10% MeOH/DCM) to obtain the desired N-(3-(8-amino-3-methylimidazo[1,5-a]pyrazin-1-yl)-2-fluorophenyl)-2,5-dichloro-3-(methoxymethyl)benzenesulfonamide (0.045 g, 11%) as an off-white solid. ¹H NMR (400 MHz, DMSO-d₆): δ 10.76 (br s, 1H), 7.88 (d, J=2.8 Hz, 1H), 7.74 (d, J=2.4 Hz, 1H), 7.50 (d, J=4.8 Hz, 1H), 7.20-7.31 (m, 3H), 7.05 (d, J=4.8 Hz, 1H), 5.88 (br s, 2H), 4.55 (s, 2H), 3.37 (s, 3H), 2.55 (s, 3H); MS (ESI) m/z: 510.0 (M+H⁺).

The following compounds are prepared essentially by method of preparation of example 48.

| Ex | | Yield | ¹H NMR | MS (m/z: |
|---|---|---|---|---|
| No | Product | (%) | (400 MHz, DMSO-d₆): δ | M + H⁺) |
| 49 | 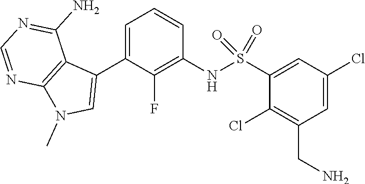 [SMILES: Cn1cc(-c2cccc(NS(=O)(=O)c3cc(Cl)cc(CN)c3Cl)c2F)c2c(N)ncnc21] | 15 | 8.15 (s, 1H), 7.91 (d, J = 2.4 Hz, 1H), 7.81 (d, J = 2.4 Hz, 1H), 7.20 (m, 2H), 7.08 (t, J = 8.0 Hz, 1H), 7.0 (t, J = 6.4 Hz, 1H), 5.70-6.05 (br m, 4H), 3.98 (s, 2H), 3.75 (s, 3H), one NH is missing, at high temp. | 495.3 |
| 50 | 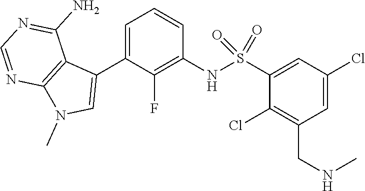 [SMILES: CNCc1cc(Cl)cc(S(=O)(=O)Nc2cccc(-c3cn(C)c4ncnc(N)c34)c2F)c1Cl] | 28 | 8.14 (s, 1H), 7.93 (d, J = 2.4 Hz, 1H), 7.76 (d, J = 2.4 Hz, 1H), 7.24 (s, 1H), 7.12 (t, J = 7.6 Hz, 1H), 7.03 (t, J = 8.0 Hz, 1H), 6.90 (br m, 1H), 5.97 (br m, 2H), 4.01 (br s, 2H), 3.73 (s, 3H), 2.45 (s, 3H). two NH protons are missing. | 509.3 |

### Oxidation

### Preparation of example 51: N-(3-(4-amino-7-methyl-7H-pyrrolo[2,3-d]pyrimidin-5-yl)-2-fluorophenyl)-2,5-dichloro-3-formylbenzenesulfonamide

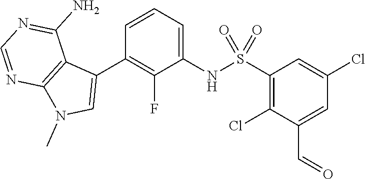 [SMILES: Cn1cc(-c2cccc(NS(=O)(=O)c3cc(Cl)cc(C=O)c3Cl)c2F)c2c(N)ncnc21]

A solution of N-(3-(4-amino-7-methyl-7H-pyrrolo[2,3-d]pyrimidin-5-yl)-2-fluorophenyl)-2,5-dichloro-3-(hydroxymethyl)benzenesulfonamide (synthesized by those skilled in the art, 0.5 g, 1.0 mmol) in THF (20 mL) was treated with MnO₂ (1.3 g, 15 mmol). The reaction mixture was stirred at rt for 3 days. The reaction was diluted with EtOAc and filtered through a pad of celite. The filtrate was concentrated under reduced pressure to obtain N-(3-(4-amino-7-methyl-7H-pyrrolo[2,3-d]pyrimidin-5-yl)-2-fluorophenyl)-2,5-dichloro-3-formylbenzenesulfonamide (crude, 100%). MS (ESI) m/z: 494.0 (M+H⁺).

### Formation of Oxime

### Preparation of example 52: (Z)—N-(3-(4-amino-7-methyl-7H-pyrrolo[2,3-d]pyrimidin-5-yl)-2-fluorophenyl)-2,5-dichloro-3-((hydroxyimino)methyl)benzenesulfonamide 11597

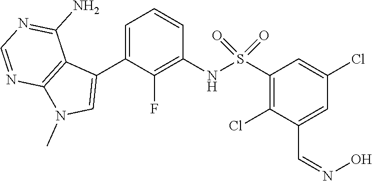 [SMILES: Cn1cc(-c2cccc(NS(=O)(=O)c3cc(Cl)cc(/C=N\O)c3Cl)c2F)c2c(N)ncnc21]

A solution of crude N-(3-(4-amino-7-methyl-7H-pyrrolo[2,3-d]pyrimidin-5-yl)-2-fluorophenyl)-2,5-dichloro-3-formylbenzenesulfonamide (51, 0.49 g, 1.0 mmol) in EtOH (20 mL) was treated with NH₂OH HCl (0.35 g, 5.0 mmol). The reaction mixture was stirred at rt overnight. The mixture was concentrated under reduced pressure and then the residue was purified by reverse-phase column chromatography (10-100% MeCN (0.1% TFA)/water. Fractions containing product were combined and neutralized with sat'd NaHCO₃ solution. The solution was extracted with 5% MeOH/DCM. The organic layer was dried over anhydrous MgSO₄, filtered, and concentrated under reduced pressure to obtain (Z)—N-(3-(4-amino-7-methyl-7H-pyrrolo[2,3-d]pyrimidin-5-yl)-2-fluorophenyl)-2,5-dichloro-3-((hydroxyimino)methyl)benzenesulfonamide (0.12 g, 21%) as a pale-yellow solid. ¹H NMR (400 MHz, DMSO-d₆): δ 12.11 (s, 1H), 10.76 (br s, 1H), 8.43 (s, 1H), 8.14 (s, 1H), 7.96 (s, 1H), 7.26 (d, J=5.4 Hz, 1H), 7.14-7.22 (m, 4H), 6.00 (br s, 2H), 3.73 (s, 3H); MS (ESI) m/z: 509.0 (M+H⁺).

The following compounds are prepared essentially by method of preparation of example 52.

| Ex | | Yield | ¹H NMR | MS (m/z: |
|---|---|---|---|---|
| No | Product | (%) | (400 MHz, DMSO-d₆): δ | M + H⁺) |
| 53 | 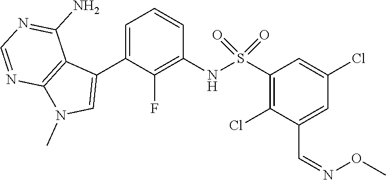 [SMILES: CO/N=C\c1cc(Cl)cc(S(=O)(=O)Nc2cccc(-c3cn(C)c4ncnc(N)c34)c2F)c1Cl] | 5 | 10.72 (s, 1H), 8.44 (s, 1H), 8.08 (s, 1H), 7.87- 7.98 (m, 2H), 7.20 (s, 1H), 7.12 (d, J = 7.5 Hz, 3H), 5.93 (s, 2H), 3.91 (s, 3H), 3.66(s, 3H). | 523.0 |

Biochemical assay for GCN2

Activity of GCN2 kinase was determined using a TR-FRET kinase activity assay (e.g., Analytical Biochemistry, 2006, 356, 108-116). Assays were conducted in 384-well plates (13 μL assay volume) using 2 nM GCN2 (Carna Biosciences), 130 nM GFP-EIf2α (Invitrogen), 0.2 mg/mL E. coli tRNA (sigma) and 1 mM ATP in kinase buffer (Invitrogen). Inhibition of GCN2 was measured by adding serial diluted test compound (final assay concentration of 0.5% DMSO) followed by a 3-hour incubation. Tb-peIF2α (pSer52) antibody (Invitrogen) (2 nM final assay concentration) in kinase buffer containing EDTA (final assay concentration of 20 mM) was added. After a 60 min incubation at room temperature, TR-FRET was monitored using an excitation wavelength of 340 nm and emission wavelengths of 490 nm and 520 nm. The emission ratio (520/490) at each compound concentration of was converted to percent inhibition using controls (i.e., reaction with no test compound and reaction with a known inhibitor) and IC₅₀ values were calculated by fitting a four-parameter sigmoidal curve to the data using Prism (GraphPad software).

GCN2 protein sequence (residues 1-1649; G556E with a
N-terminal GST tag)
SEQ ID NO: 1
MAPILGYWKIKGLVQPTRLLLEYLEEKYEEHLYERDEGDKWRNKKFELGLEFPNLPY
YIDGDVKLTQSMAIIRYIADKHNMLGGCPKERAEISMLEGAVLDIRYGVSRIAYSKDF
ETLKVDFLSKLPEMLKMFEDRLCHKTYLNGDHVTHPDFMLYDALDVVLYMDPMCL
DAFPKLVCFKKRIEAIPQIDKYLKSSKYIAWPLQGWQATFGGGDHPPKSDLEVLFQG
PLGAMGSGIQRPTSTSSLVMAGGRGAPGRGRDEPPESYPQRQDHELQALEAIYGADF
QDLRPDACGPVKEPPEINLVLYPQGLTGEEVYVKVDLRVKCPPTYPDVVPEIELKNA
KGLSNESVNLLKSRLEELAKKHCGEVMIFELAYHVQSFLSEHNKPPPKSFHEEMLER
RAQEEQQRLLEAKRKEEQEQREILHEIQRRKEEIKEEKKRKEMAKQERLEIASLSNQD
HTSKKDPGGHRTAAILHGGSPDFVGNGKHRANSSGRSRRERQYSVCNSEDSPGSCEI
LYFNMGSPDQLMVHKGKCIGDEQLGKLVYNALETATGGFVLLYEWVLQWQKKMG
PFLTSQEKEKIDKCKKQIQGTETEFNSLVKLSHPNVVRYLAMNLKEQDDSIVVDILVE
HISGVSLAAHLSHSGPIPVHQLRRYTAQLLSGLDYLHSNSVVHKVLSASNVLVDAEG
TVKITDYSISKRLADICKEDVFEQTRVRFSDNALPYKTGKKGDVWRLGLLLLSLSQG
QECGEYPVTIPSDLPADFQDFLKKCVCLDDKERWSPQQLLKHSFINPQPKMPLVEQSP
EDSGGQDYVETVIPSNRLPSAAFFSETQRQFSRYFIEFEELQLLGKGAFGAVIKVQNK
LDGCCYAVKRIPINPASRQFRRIKGEVTLLSRLHHENIVRYYNAWIERHERPAGPGTP
PPDSGPLAKDDRAARGQPASDTDGLDSVEAAAPPPILSSSVEWSTSGERSASARFPAT
GPGSSDDEDDDEDEHGGVFSQSFLPASDSESDIIFDNEDENSKSQNQDEDCNEKNGC
HEESEPSVTTEAVHYLYIQMEYCEKSTLRDTIDQGLYRDTVRLWRLFREILDGLAYIHE
KGMIHRDLKPVNIFLDSDDHVKIGDFGLATDHLAFSADSKQDDQTGDLIKSDPSGHL
TGMVGTALYVSPEVQGSTKSAYNQKVDLFSLGIIFFEMSYHPMVTASERIFVLNQLR
DPTSPKFPEDFDDGEHAKQKSVISWLLNHDPAKRPTATELLKSELLPPPQMEESELHE
VLHHTLTNVDGKAYRTMMAQIFSQRISPAIDYTYDSDILKGNFSIRTAKMQQHVCETI
IRIFKRHGAVQLCTPLLLPRNRQIYEHNEAALFMDHSGMLVMLPFDLRIPFARYVAR
NNILNLKRYCIERVFRPRKLDRFHPKELLECAFDIVTSTTNSFLPTAEIIYTIYEIIQEFPA
LQERNYSIYLNHTMLLKAILLHCGIPEDKLSQVYIILYDAVTEKLTRREVEAKFCNLSL
SSNSLCRLYKFIEQKGDLQDLMPTINSLIKQKTGIAQLVKYGLKDLEEVVGLLKKLGI
KLQVLINLGLVYKVQQHNGIIFQFVAFIKRRQRAVPEILAAGGRYDLLIPQFRGPQAL
GPVPTAIGVSIAIDKISAAVLNMEESVTISSCDLLVVSVGQMSMSRAINLTQKLWTAG
ITAEIMYDWSQSQEELQEYCRHHEITYVALVSDKEGSHVKVKSFEKERQTEKRVLET
ELVDHVLQKLRTKVTDERNGREASDNLAVQNLKGSFSNASGLFEIHGATVVPIVSVL
APEKLSASTRRRYETQVQTRLQTSLANLHQKSSEIEILAVDLPKETILQFLSLEWDAD
EQAFNTTVKQLLSRLPKQRYLKLVCDEIYNIKVEKKVSVLFLYSYRDDYYRILF

### Biochemical Assay for PERK

Activity of PERK kinase was determined spectroscopically using a coupled pyruvate kinase/lactate dehydrogenase assay that continuously monitors the ATP hydrolysis-dependent oxidation of NADH (Science, 2000, 289, 1938-1942). Assays were conducted in 384-well plates (100 μL final volume) using 10 nM PERK (from Beryllium), 0.25 mg/mL Myelin Basic Protein substrate, 1.5 units pyruvate kinase, 2.1 units lactate dehydrogenase, 1 mM phosphoenol pyruvate, 0.28 mM NADH and 1 mM ATP in assay buffer (100 mM Tris, pH 7.5, 15 mM MgCl₂, 0.5 mM DTT, 0.004% (w/v) BSA, and 0.004% Triton X-100). Inhibition of PERK was measured by adding serial diluted test compound (final assay concentration of 1% DMSO). A decrease in absorption at 340 nm was monitored continuously for 6 hours at 30° C. on a multi-mode microplate reader (BioTek). The reaction rate was calculated using the 2-3 h time frame. The reaction rate at each concentration of compound was converted to percent inhibition using controls (i.e., reaction with no test compound and reaction with a known inhibitor) and IC₅₀ values were calculated using software routines in Prism (GraphPad software).

PERK protein sequence (residues 563-1115; Sequence ID: NM 004836)
SEQ ID NO: 2
MSPILGYWKIKGLVQPTRLLLEYLEEKYEEHLYERDEGDKWRNKKFELGLEFPNLPY
YIDGDVKLTQSMAIIRYIADKHNMLGGCPKERAEISMLEGAVLDIRYGVSRIAYSKDF
ETLKVDFLSKLPEMLKMFEDRLCHKTYLNGDHVTHPDFMLYDALDVVLYMDPMCL
DAFPKLVCFKKRIEAIPQIDKYLKSSKYIAWPLQGWQATFGGGDHPPKSDLVPRGSK
YDSVSGEANDSSWNDIKNSGYISRYLTDFEPIQCLGRGGFGVVFEAKNKVDDCNYAI
KRIRLPNRELAREKVMREVKALAKLEHPGIVRYFNAWLEAPPEKWQEKMDEIWLKD
ESTDWPLSSPSPMDAPSVKIRRMDPFSTKEHIEIIAPSPQRSRSFSVGISCDQTSSSESQF
SPLEFSGMDHEDISESVDAAYNLQDSCLTDCDVEDGTMDGNDEGHSFELCPSEASPY
VRSRERTSSSIVFEDSGCDNASSKEEPKTNRLHIGNHCANKLTAFKPTSSKSSSEATLSI
SPPRPTTLSLDLTKNTTEKLQPSSPKVYLYIQMQLCRKENLKDWMNGRCTIEERERS
VCLHIFLQIAEAVEFLHSKGLMHRDLKPSNIFFTMDDVVKVGDFGLVTAMDQDEEE
QTVLTPMPAYARHTGQVGTKLYMSPEQIHGNSYSHKVDIFSLGLILFELLYPFSTQME
RVRTLTDVRNLKFPPLFTQKYPCEYVMVQDMLSPSPMERPEAINIIENAVFEDLDFPG
KTVLRQRSRSLSSSGTKHSRQSNNSHSPLPSN

TABLE 1
Inhibition of biochemical activity of GCN2
and PERK kinases by exemplary compounds.

| Example | GCN2 | PERK |
|---|---|---|
| No | IC₅₀ (nM) | IC₅₀ (nM) |
| 1 | ++++ | ++ |
| 2 | ++ | + |
| 3 | ++ | + |
| 4 | + | + |
| 5 | ++++ | +++ |
| 6 | ++++ | + |
| 7 | +++ | + |
| 8 | +++ | + |
| 9 | ++ | + |
| 10 | ++++ | ++ |
| 11 | ++++ | ++ |
| 12 | + | + |
| 13 | ++ | + |
| 14 | + | + |
| 15 | ++++ | ++ |
| 16 | + | + |
| 17 | + | + |
| 18 | + | + |
| 19 | + | + |
| 20 | + | + |
| 21 | + | + |
| 22 | + | + |
| 23 | ++ | + |
| 24 | ++++ | ++ |
| 25 | ++++ | + |
| 26 | + | + |
| 27 | + | + |
| 28 | ++++ | +++ |
| 29 | ++++ | ++++ |
| 30 | ++++ | ++ |
| 31 | ++++ | ++++ |
| 32 | | +++ |
| 33 | ++++ | +++ |
| 34 | ++ | + |
| 35 | ++++ | ++ |
| 36 | ++++ | ++ |
| 37 | ++ | + |
| 38 | ++++ | + |
| 39 | ++++ | ++ |
| 40 | ++++ | + |
| 41 | ++++ | + |
| 42 | ++ | + |
| 43 | ++++ | + |
| 44 | ++++ | + |
| 45 | ++++ | + |
| 48 | +++ | + |
| 49 | ++++ | ++ |
| 50 | ++++ | +++ |
| 52 | +++ | + |
| 53 | ++++ | + |

For Table 1, “+” refers to an IC₅₀ less than or equal to 100 nM; “++” refers to an IC₅₀ greater than 100 nM and less than or equal to 500 nM; “+++” refers to an IC₅₀ greater than 500 nM and less than or equal to 1000 nM; and “++++” refers to an IC₅₀ greater than 1000 nM and less than or equal to 10000 nM.

### CCRF-CEM ASNase Cell Proliferation Assay, a Phenotypic Assay for Cellular Inhibition of GCN2.

CCRF-CEM leukemia cells (catalog #CCL-116) were obtained from the American Type Culture Collect (ATTC, Manassas, VA). Cells were grown in RPMI-1640 medium supplemented with 10% heat-inactivated fetal bovine serum (Invitrogen, Carlsbad, CA) and 1% Penicillin/Streptomycin/L-Glutamine at 37° C., 5% CO₂, and 95% humidity. Cells were expanded until reaching one million cells per mL at which time they are subcultured or harvested for assay use. Ten thousand cells per well in 200 μL RPMI-1640 supplemented with 10% heat-inactivated fetal bovine serum and 1% Penicillin/Streptomycin were dispensed into a 96-well black clear bottom plate. A serial dilution of test compound and 1 mU/mL ASNase was added in triplicate and plates were incubated for 72 h at 37° C., 5% CO₂, and 95% humidity. At the end of the incubation, 40 μL of a 440 mM solution of resazurin (Sigma, St. Louis, MO) in PBS was added to each well of the plate and plates were incubated for an additional 6 h at 37° C., 5% CO₂, and 95% humidity. Plates were read on a Synergy2 or equivalent reader (Biotek, Winooski VT) using an excitation of 540 nm and an emission of 600 nm. Data was analyzed using GraphPad Prism software (GraphPad, San Diego, CA) to calculate IC₅₀ values.

### CCRF-CEM TG ATF4 ELISA, a Phenotypic Assay for Cellular Inhibition of PERK Preactivated by Thapsigargin (TG)

CCRF-CEM leukemia cells (catalog #CCL-116) were obtained from the American Type Culture Collection (ATTC, Manassas, VA). Briefly, cells were grown in RPMI-1640 medium supplemented with 10% heat-inactivated fetal bovine serum (Invitrogen, Carlsbad, CA) and 1% Penicillin/Streptomycin/L-Glutamine at 37° C., 5% CO₂, and 95% humidity. Cells were expanded until reaching one million cells per mL at which time they were subcultured or harvested for assay use. One million five hundred thousand cells per well in 1 mL complete growth medium were dispensed into 12-well plates and incubated overnight. A serial dilution of test compound was added and cells were incubated at 37° C., 5% CO₂, and 95% for three hours, then 1 μM thapsigargin was added and cells were incubated for an additional hour at 37° C., 5% CO₂, and 95%. Cells were lysed then ATF4 levels were measured using an ELISA assay (Proteintech, Rosemont, IL). Absorbance was measured at 450 nM and 544 nM using a Synergy2 or equivalent reader (Biotek, Winooski VT). Data was analyzed using PRISM software (Graphpad, San Diego, CA) to calculate IC₅₀ values.

TABLE 2
Inhibition of proliferation of ASNase treated
CCRF-CEM cells, and ATF4 in Thapsigargin stimulated
CCRF-CEM cells by exemplary compounds.

| | CCRF-CEM | CCRF-CEM |
|---|---|---|
| Example | ASNase Cell | Thapsigargin |
| No | Proliferation | ATF4 ELISA |
| 1 | +++ | +++ |
| 2 | ++ | ++ |
| 3 | ++ | ++ |
| 4 | ++++ | ++++ |
| 5 | ++++ | ++++ |
| 6 | ++++ | ++++ |
| 7 | ++++ | ++++ |
| 8 | +++ | ++ |
| 9 | ++ | ++ |
| 10 | ++++ | ++++ |
| 11 | ++++ | +++ |
| 12 | ++ | +++ |
| 13 | ++ | ++ |
| 14 | + | ++ |
| 15 | ++++ | ++++ |
| 16 | + | ++ |
| 17 | ++ | ++ |
| 18 | ++ | +++ |
| 19 | ++ | +++ |
| 20 | + | + |
| 21 | ++ | ++ |
| 22 | ++ | ++ |
| 23 | ++ | ++ |
| 24 | ++++ | ++++ |
| 25 | ++++ | ++ |
| 26 | ++ | ++ |
| 27 | ++ | ++ |
| 28 | ++++ | ++++ |
| 29 | ++++ | ++++ |
| 30 | ++++ | ++++ |
| 31 | ++++ | ++++ |
| 32 | ++++ | ++++ |
| 33 | ++ | ++ |
| 34 | +++ | ++ |
| 35 | ++++ | ++++ |
| 36 | ++++ | ++++ |
| 37 | +++ | ++ |
| 38 | ++++ | ++ |
| 39 | ++++ | ++++ |
| 40 | ++++ | ++++ |
| 41 | ++++ | ++ |
| 42 | ++++ | ++++ |
| 43 | ++++ | ++++ |
| 44 | ++++ | +++ |
| 45 | ++++ | ++ |
| 48 | ++++ | ++++ |
| 49 | ++++ | +++ |
| 50 | ++++ | ++++ |
| 52 | ++++ | ++ |
| 53 | ++++ | ++++ |

For Table 2, “+” refers to an IC₅₀ less than or equal to 100 nM; “++” refers to an IC₅₀ greater than 100 nM and less than or equal to 500 nM; “+++” refers to an IC₅₀ greater than 500 nM and less than or equal to 1000 nM; and “++++” refers to an IC₅₀ greater than 1000 nM and less than or equal to 10000 nM.

### H929 ATF4 Elisa Assay

H929 multiple myeloma cells (catalog #CRL-9068) were obtained from the American Type Culture Collection (ATTC, Manassas, VA). Briefly, cells were grown in RPMI-1640 medium supplemented with 20% heat-inactivated fetal bovine serum (catalog #A3840002, ThermoFisher Scientific, Waltham, MA), 1% Penicillin/Streptomycin/L-Glutamine (catalog #10378016, ThermoFisher Scientific, Waltham, MA), and 0.05 mM 2-mercaptoethanol (catalog #21985-023, ThermoFisher Scientific, Waltham, MA) at 37° C., 5% CO₂, and 95% humidity. Cells were expanded until reaching one million five hundred thousand cells per mL at which time they were sub-cultured or harvested for assay use. One million five hundred thousand cells per well in 1 mL complete growth medium were dispensed into 12-well plates and incubated overnight. A serial dilution of test compound was added, and cells were incubated at 37° C., 5% CO₂, and 95% for four hours. Cells were lysed then ATF4 levels were measured using an ELISA assay (Proteintech, Rosemont, IL). Absorbance is measured at 450 nM and 544 nM using a Synergy2 or equivalent reader (Biotek, Winooski VT). Data were analyzed using PRISM software (Graphpad, San Diego, CA) to calculate fold stimulation of cellular ATF4 relative to vehicle treated control.

TABLE 3
Stimulation of ATF4 in H929 Multiple
Myeloma cells by exemplary compounds.

| Example No | H929 NS ATF4 ELISA |
|---|---|
| 1 | ++++ |
| 2 | +++ |
| 3 | ++ |
| 4 | + |
| 9 | +++ |
| 12 | ++++ |
| 13 | +++ |
| 14 | ++ |
| 16 | +++ |
| 17 | +++ |
| 18 | ++ |
| 19 | ++ |
| 20 | ++ |
| 21 | +++ |
| 22 | ++++ |
| 21 | +++ |
| 26 | ++ |
| 34 | ++++ |
| 35 | + |
| 38 | + |
| 43 | + |
| 52 | ++++ |

For Table 3, “+” refers to an ATF4 stim less than or equal to 5-fold; “++” refers to an ATF4 stim greater than 5-fold and less than or equal to 10-fold; “+++” refers to an ATF4 stim greater than 10-fold and less than or equal to 20-fold; “++++” refers to an ATF4 stim greater than 20-fold.

### EQUIVALENTS

While specific embodiments have been discussed, the above specification is illustrative and not restrictive. Many variations of the embodiments will become apparent to those skilled in the art upon review of this specification. The full scope of what is disclosed should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained.

What is claimed is:

1. A compound represented by Formula I-A: 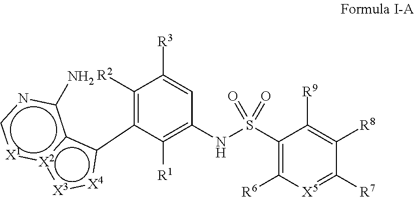
or a pharmaceutically acceptable salt, enantiomer, stereoisomer, or tautomer thereof, wherein:
X¹ and X⁴ are each independently selected from the group consisting of CH and N;
X² is selected from group consisting of C and N;
X³ is selected from the group consisting of CR⁴ and NR⁴;
provided that not more than two of X¹, X², X³, and X⁴ is N;
X⁵ is selected from the group consisting of CR⁵ and N;
R¹ and R² are each independently selected from the group consisting of H, alkoxy, alkyl, cyano, haloalkoxy, haloalkyl, and halogen;
R³ is selected from the group consisting of H, alkyl, alkoxy, cyano, and halogen;
R⁴ is selected from the group consisting of H, alkyl, alkenyl, alkenylalkyl, alkynyl, alkynylalkyl, cycloalkyl, cycloalkylalkyl, alkoxyalkyl, cycloalkenyl, alkylamino, amide, thioalkyl, heterocyclyl, heterocyclylalkyl, aryl, heteroaryl, and heteroarylalkyl;
R⁵ is selected from the group consisting of H, hydroxyalkyl, hydroxycycloalkyl, alkoxyalkyl, alkoxycycloalkyl, halogen, fluoroalkyl, cyano, alkoxy, amine, aminoalkyl, aminocycloalkyl, aminocarbonyl, acylamino, acyloxyalkyl, hydroxyimino, alkoxyimino, alkylamino, cyanoalkyl, alkyl, cycloalkyl, cycloalkoxy, cycloalkylamino, alkoxycarbonyl, and heterocyclylalkyl;
R⁶, R⁷, and R⁸ are each independently selected from the group consisting of H, alkyl, alkoxy, hydroxy, cyano, halogen, alkylamino, cycloalkoxy, amine, halogen, haloalkoxy, haloalkyl, amide, alkoxycarbonyl, and hydroxyalkyl; and
R⁹ is selected from the group consisting of H, halogen, and alkyl;
with the proviso that:
i) when X² is C, 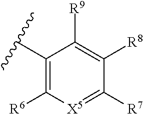
is not 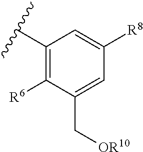
wherein R⁶ is selected from the group consisting of halogen, alkoxy and alkyl;
R⁸ is selected from the group consisting of H, halogen, and alkyl; and
R¹⁰ is selected from the group consisting of H, alkyl, and acyl;
ii) when X² is C and X³ is NR⁴, 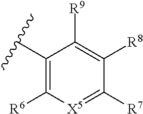
is not 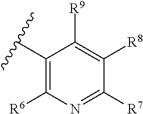
wherein R⁶ is H;
R⁷ is selected from the group consisting of H, Cl, and OCH₃;
R⁸ is H or Br; and
R⁹ is H; and
iii) when X² is C and X³ is NR⁴, 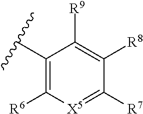
is not 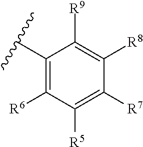
wherein R⁵ is selected from the group consisting of H, F, Cl, CH₃, OCH₃, CF₃, and CN;
R⁶ is H or F;
R⁷ is selected from the group consisting of H, F, Cl, Br, I, CH₃, OCH₃, OCH₂CH₃, OCH(CH₃)₂, CF₃, OH, and OCF₃;
R⁸ is selected from the group consisting of H, F, Cl, CH₃, OCH₃, CF₃, and CN; and
R⁹ is H or F.

2. The compound of claim 1, wherein at least one of R¹, R², and R³ is halogen.

3. The compound of claim 1 or 2, wherein at least one of R¹, R², and R³ is fluoro.

4. The compound of any one of claims 1-3, wherein R¹ is fluoro.

5. The compound of any one of claims 1-4, wherein X¹ is N.

6. The compound of any one of claims 1-4, wherein X² is N.

7. A compound represented by Formula I-B: 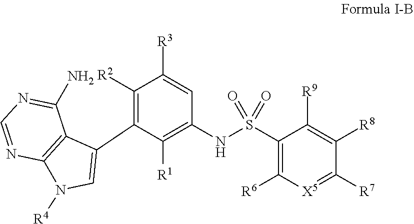
or a pharmaceutically acceptable salt, enantiomer, stereoisomer, or tautomer thereof, wherein:
X⁵ is selected from the group consisting of CR⁵ and N;
R¹ and R² are each independently selected from the group consisting of H, alkoxy, alkyl, cyano, haloalkoxy, haloalkyl, and halogen;
R³ is selected from the group consisting of H, alkyl, and halogen;
R⁴ is selected from the group consisting of H, alkyl, alkenyl, alkenylalkyl, alkynyl, alkynylalkyl, cycloalkyl, cycloalkylalkyl, alkoxyalkyl, cycloalkenyl, alkylamino, amide, thioalkyl, heterocyclyl, heterocyclylalkyl, aryl, heteroaryl, and heteroarylalkyl;
R⁵ is selected from the group consisting of H, hydroxyalkyl, hydroxycycloalkyl, alkoxyalkyl, alkoxycycloalkyl, halogen, fluoroalkyl, cyano, alkoxy, amine, aminoalkyl, aminocycloalkyl, aminocarbonyl, acylamino, acyloxyalkyl, hydroxyimino, alkoxyimino, alkylamino, cyanoalkyl, alkyl, cycloalkyl, cycloalkoxy, cycloalkylamino, alkoxycarbonyl, and heterocyclylalkyl;
R⁶, R⁷, and R⁸ are each independently selected from the group consisting of H, alkyl, alkoxy, hydroxy, cyano, halogen, alkylamino, cycloalkoxy, amine, halogen, haloalkoxy, haloalkyl, amide, alkoxycarbonyl, and hydroxyalkyl; and
R⁹ is selected from the group consisting of H, halogen, and alkyl;
with the proviso that:
i) 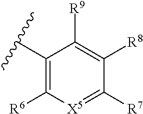
is not 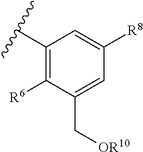
wherein R⁶ is selected from the group consisting of halogen, alkoxy and alkyl;
R⁸ is selected from the group consisting of H, halogen and alkyl; and
R¹⁰ is selected from the group consisting of H, alkyl, and acyl;
ii) 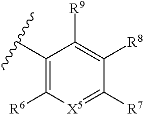
is not 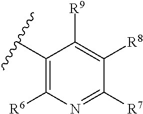
wherein R⁶ is H;
R⁷ is selected from the group consisting of H, Cl, and OCH₃;
R⁸ is H or Br; and
R⁹ is H; and
iii) 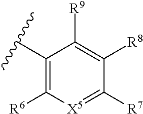
is not 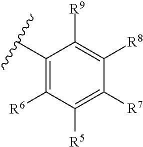
wherein R⁵ is selected from the group consisting of H, F, Cl, CH₃, OCH₃, CF₃, and CN;
R⁶ is H or F;
R⁷ is selected from the group consisting of H, F, Cl, Br, I, CH₃, OCH₃, OCH₂CH₃, OCH(CH₃)₂, CF₃, OH, and OCF₃;
R⁸ is selected from the group consisting of H, F, Cl, CH₃, OCH₃, CF₃, and CN; and
R⁹ is H or F.

8. A compound represented by Formula I-C: 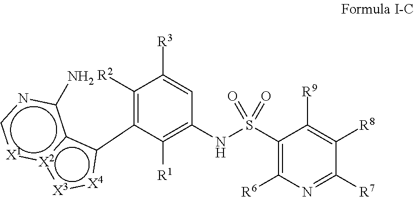
or a pharmaceutically acceptable salt, enantiomer, stereoisomer, or tautomer thereof, wherein:
X¹ and X⁴ are each independently selected from the group consisting of CH and N;
X² is selected from group consisting of C and N;
X³ is selected from the group consisting of CR⁴ and NR⁴;
provided that not more than two of X¹, X², X³, and X⁴ is N;
R¹ and R² are each independently selected from the group consisting of H, alkoxy, alkyl, cyano, haloalkoxy, haloalkyl, and halogen;
R³ is selected from the group consisting of H, alkyl, and halogen;
R⁴ is selected from the group consisting of H, alkyl, alkenyl, alkenylalkyl, alkynyl, alkynylalkyl, cycloalkyl, cycloalkylalkyl, alkoxyalkyl, cycloalkenyl, alkylamino, amide, thioalkyl, heterocyclyl, heterocyclylalkyl, aryl, heteroaryl, and heteroarylalkyl;
R⁶, R⁷, and R⁸ are each independently selected from the group consisting of H, alkyl, alkoxy, hydroxy, cyano, halogen, alkylamino, cycloalkoxy, amine, halogen, haloalkoxy, haloalkyl, amide, alkoxycarbonyl, and hydroxyalkyl; and
R⁹ is selected from the group consisting of H, halogen, and alkyl.
with proviso that
when X² is C, X³ is NR⁴, R⁶ is H, R⁸ is H or Br, and R⁹ is H, R⁷ is not H, Cl, or OCH₃.

9. The compound of claim 8, wherein X¹ is N.

10. The compound of claim 8, wherein X² is N.

11. A compound represented by Formula I-D: 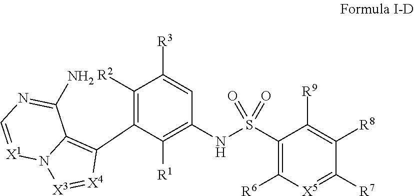
or a pharmaceutically acceptable salt, enantiomer, stereoisomer, or tautomer thereof, wherein:
X¹ and X⁴ are each independently selected from the group consisting of CH and N;
X³ is selected from the group consisting of CR⁴ and NR⁴;
provided that not more than one of X¹, X³, and X⁴ is N;
X⁵ is selected from the group consisting of CR⁵ and N;
R¹ and R² are each independently selected from the group consisting of H, alkoxy, alkyl, cyano, haloalkoxy, haloalkyl, and halogen;
R³ is selected from the group consisting of H, alkyl, and halogen;
R⁴ is selected from the group consisting of H, alkyl, alkenyl, alkenylalkyl, alkynyl, alkynylalkyl, cycloalkyl, cycloalkylalkyl, alkoxyalkyl, cycloalkenyl, alkylamino, amide, thioalkyl, heterocyclyl, heterocyclylalkyl, aryl, heteroaryl, and heteroarylalkyl;
R⁵ is selected from the group consisting of H, hydroxyalkyl, hydroxycycloalkyl, alkoxyalkyl, alkoxycycloalkyl, halogen, fluoroalkyl, cyano, alkoxy, amine, aminoalkyl, aminocycloalkyl, aminocarbonyl, acylamino, acyloxyalkyl, hydroxyimino, alkoxyimino, alkylamino, cyanoalkyl, alkyl, cycloalkyl, cycloalkoxy, cycloalkylamino, alkoxycarbonyl, and heterocyclylalkyl;
R⁶, R⁷, and R⁸ are each independently selected from the group consisting of H, alkyl, alkoxy, hydroxy, cyano, halogen, alkylamino, cycloalkoxy, amine, halogen, haloalkoxy, haloalkyl, amide, alkoxycarbonyl, and hydroxyalkyl; and
R⁹ is selected from the group consisting of H, halogen, and alkyl.

12. The compound of claim 11, wherein X¹ is CH, X³ is CR⁴, and X⁴ is N.

13. A compound represented by Formula I-E: 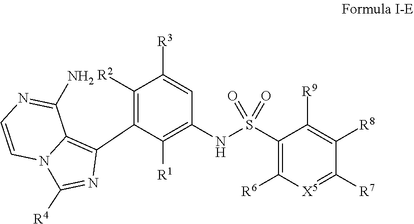
or a pharmaceutically acceptable salt, enantiomer, stereoisomer, or tautomer thereof, wherein:
X⁵ is selected from the group consisting of CR⁵ and N;
R¹ and R² are each independently selected from the group consisting of H, alkoxy, alkyl, cyano, haloalkoxy, haloalkyl, and halogen;
R³ is selected from the group consisting of H, alkyl, and halogen;
R⁴ is selected from the group consisting of H, alkyl, alkenyl, alkenylalkyl, alkynyl, alkynylalkyl, cycloalkyl, cycloalkylalkyl, alkoxyalkyl, cycloalkenyl, alkylamino, amide, thioalkyl, heterocyclyl, heterocyclylalkyl, aryl, heteroaryl, and heteroarylalkyl;
R⁵ is selected from the group consisting of H, hydroxyalkyl, hydroxycycloalkyl, alkoxyalkyl, alkoxycycloalkyl, halogen, fluoroalkyl, cyano, alkoxy, amine, aminoalkyl, aminocycloalkyl, aminocarbonyl, acylamino, acyloxyalkyl, hydroxyimino, alkoxyimino, alkylamino, cyanoalkyl, alkyl, cycloalkyl, cycloalkoxy, cycloalkylamino, alkoxycarbonyl, and heterocyclylalkyl;
R⁶, R⁷, and R⁸ are each independently selected from the group consisting of H, alkyl, alkoxy, hydroxy, cyano, halogen, alkylamino, cycloalkoxy, amine, halogen, haloalkoxy, haloalkyl, amide, alkoxycarbonyl, and hydroxyalkyl; and
R⁹ is selected from the group consisting of H, halogen, and alkyl.

14. A compound represented by Formula I-F: 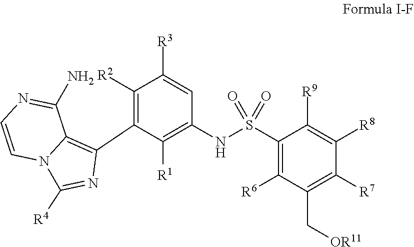
or a pharmaceutically acceptable salt, enantiomer, stereoisomer, or tautomer thereof, wherein:
R¹ and R² are each independently selected from the group consisting of H, alkoxy, alkyl, cyano, haloalkoxy, haloalkyl, and halogen;
R³ is selected from the group consisting of H, alkyl, and halogen;
R⁴ is selected from the group consisting of H, alkyl, alkenyl, alkenylalkyl, alkynyl, alkynylalkyl, cycloalkyl, cycloalkylalkyl, alkoxyalkyl, cycloalkenyl, alkylamino, amide, thioalkyl, heterocyclyl, heterocyclylalkyl, aryl, heteroaryl, and heteroarylalkyl;
R⁶, R⁷, and R⁸ are each independently selected from the group consisting of H, alkyl, alkoxy, hydroxy, cyano, halogen, alkylamino, cycloalkoxy, amine, halogen, haloalkoxy, haloalkyl, amide, alkoxycarbonyl, and hydroxyalkyl;
R⁹ is selected from the group consisting of H, halogen, and alkyl; and
R¹¹ is selected from the group consisting of H and acyl.

15. The compound of any one of claims 7-14, wherein R² is H and R³ is H.

16. The compound of any one of claims 7-14, wherein R¹ is F, R² is H, and R³ is H.

17. The compound of any one of claims 7-14, wherein R² is F and R³ is H.

18. The compound of any one of claims 7-14, wherein R² is H and R³ is F.

19. The compound of any one of claims 1-18, wherein R⁴ is selected from the group consisting of H, alkyl, (C2-C8)alkenyl, (C2-C8)alkenyl-(C1-C4)alkyl, (C2-C8)alkynyl, (C2-C8)alkynyl-(C1-C4)alkyl, (C3-C8)cycloalkyl, (C3-C8)cycloalkyl-(C1-C4)alkyl, alkoxy-(C1-C4)alkyl, (C3-C8)cycloalkenyl, (C3-C8)cycloalkenyl-(C1-C4)alkyl, alkylamino, amide, thio-(C1-C4)alkyl, heterocyclyl, heterocyclyl-(C1-C4)alkyl, aryl, heteroaryl, and heteroaryl-(C1-C4)alkyl, wherein the alkyl component of the alkylamino is optionally substituted with alkoxy.

20. The compound of any one of claims 1-19, wherein R⁴ is selected from the group consisting of H, alkyl, (C3-C8)cycloalkyl, alkylamino, amide, thio-(C1-C4)alkyl, heterocyclyl, and heteroaryl, wherein the alkyl component of the alkylamino is optionally substituted with (C1-C6)alkoxy.

21. The compound of any one of claims 1-20, wherein R⁴ is selected from the group consisting of H,

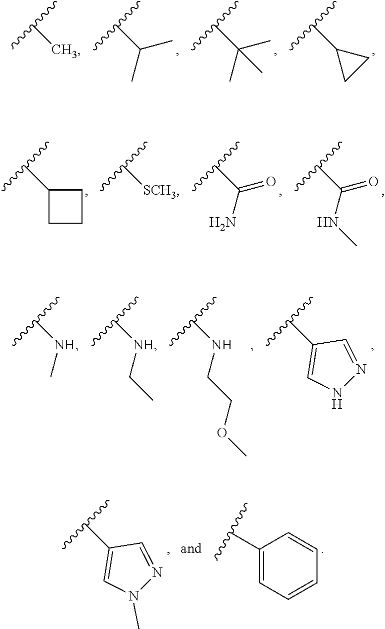

22. The compound of any one of claims 1-7, 11-13, and 15-21, wherein R⁵ is selected from the group consisting of H, alkyl, (C3-C8)cycloalkyl, alkylamino, hydroxy-(C1-C4)alkyl, hydroxy-(C3-C8)cycloalkyl, alkoxy-(C1-C4)alkyl, alkoxy-(C3-C8)cycloalkyl, halogen, fluoroalkyl, cyano, alkoxy, amine, amino-(C1-C4)alkyl, amino-(C3-C8)cycloalkyl, aminocarbonyl, acylamino, acyloxy-(C1-C4)alkyl, hydroxyimino, alkoxyimino, cyano-(C1-C4)alkyl, heterocyclyl, (C3-C8)cycloalkylamino, (C1-C4)alkoxycarbonyl, and heterocyclyl-(C1-C4)alkyl.

23. The compound of any one of claims 1-7, 11-13, and 15-22, wherein R⁵ is selected from the group consisting of H, alkyl, alkylamino, hydroxy-(C1-C4)alkyl, alkoxy-(C1-C4)alkyl, halogen, fluoroalkyl, cyano, alkoxy, amine, amino-(C1-C4)alkyl, acyloxy-(C1-C4)alkyl, hydroxyimino, alkoxyimino, cyano-(C1-C4)alkyl, heterocyclyl, and alkoxycarbonyl.

24. The compound of any one of claims 1-7, 11-13, and 15-23, wherein R⁵ is selected from the group consisting of H, fluoro, chloro, bromo, CF3,

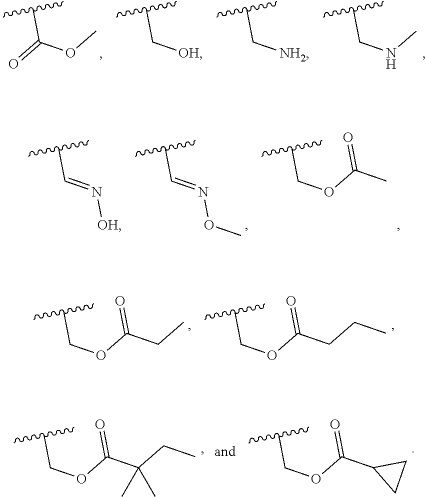

25. The compound of any one of claims 1-24, wherein R⁶, R⁷, and R⁸ are each independently selected from the group consisting of H, alkyl, alkoxy, hydroxy, cyano, halogen, haloalkyl, alkylamino, (C3-C8)cycloalkoxy, amine, halogen, haloalkoxy, haloalkyl, amide, alkoxycarbonyl, and hydroxy-(C1-C4)alkyl.

26. The compound of any one of claims 1-25, wherein R⁶, R⁷, and R⁸ are each independently selected from the group consisting of H, alkyl, alkoxy, hydroxy, halogen, and hydroxy-(C1-C4)alkyl.

27. The compound of any one of claims 1-26, wherein R⁶ is selected from the group consisting of H, methyl, methoxy, fluoro, and chloro.

28. The compound of any one of claims 1-27, wherein R⁷ is selected from the group consisting of H, methoxy, fluoro, bromo, and

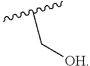 [SMILES: *CO]

29. The compound of any one of claims 1-28, wherein R⁹ is selected from the group consisting of H, halogen, and alkyl.

30. The compound of any one of claims 1-29, wherein R⁹ is selected from the group consisting of H and fluoro.

31. The compound of claim 14, wherein R¹¹ is selected from the group consisting of H,

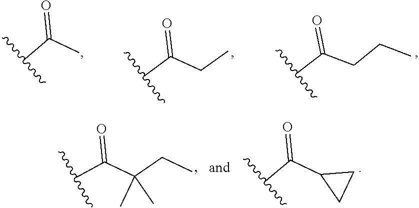

32. The compound of claim 14, wherein R¹¹ is selected from the group consisting of H, fluoro, and chloro.

33. A compound selected from the group consisting of: 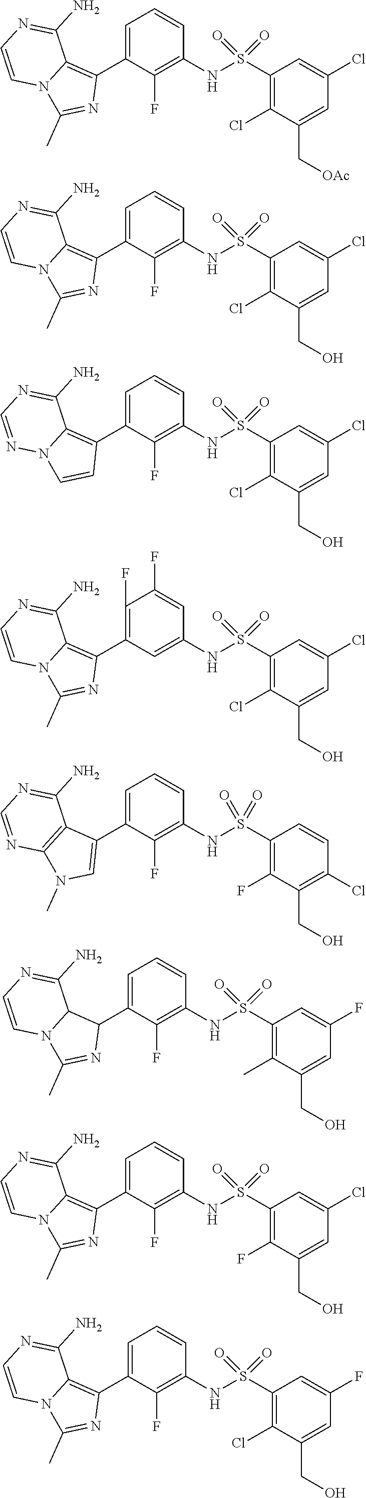 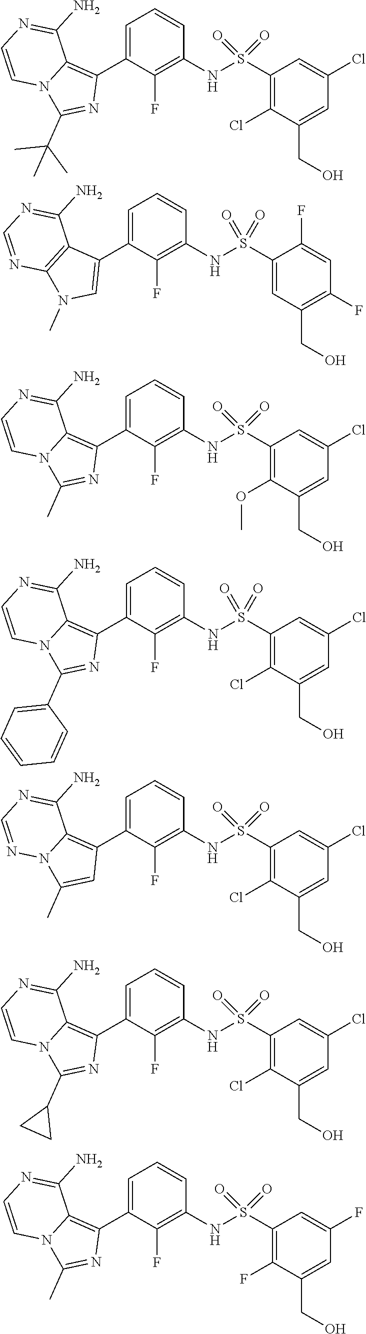 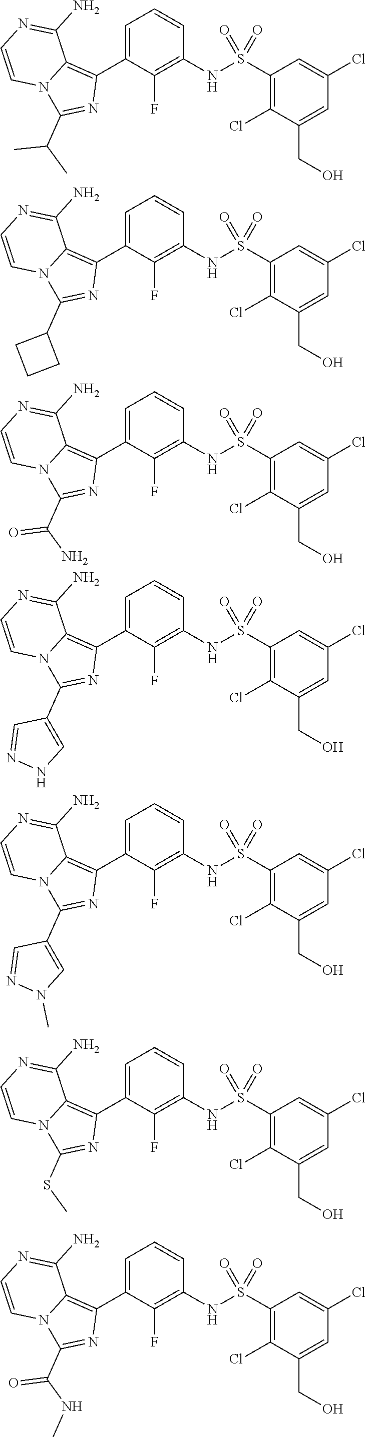 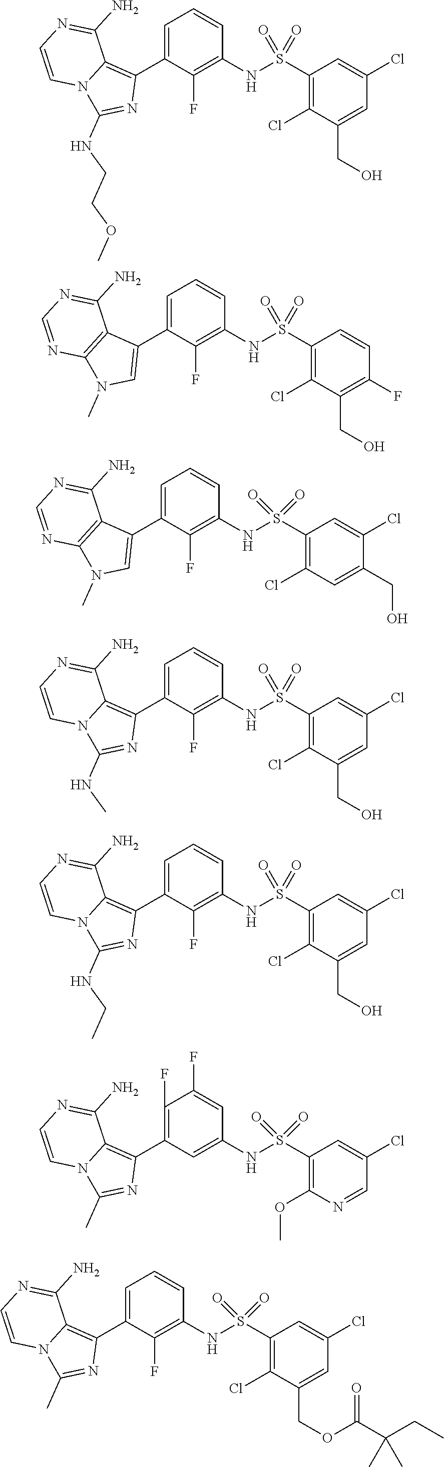 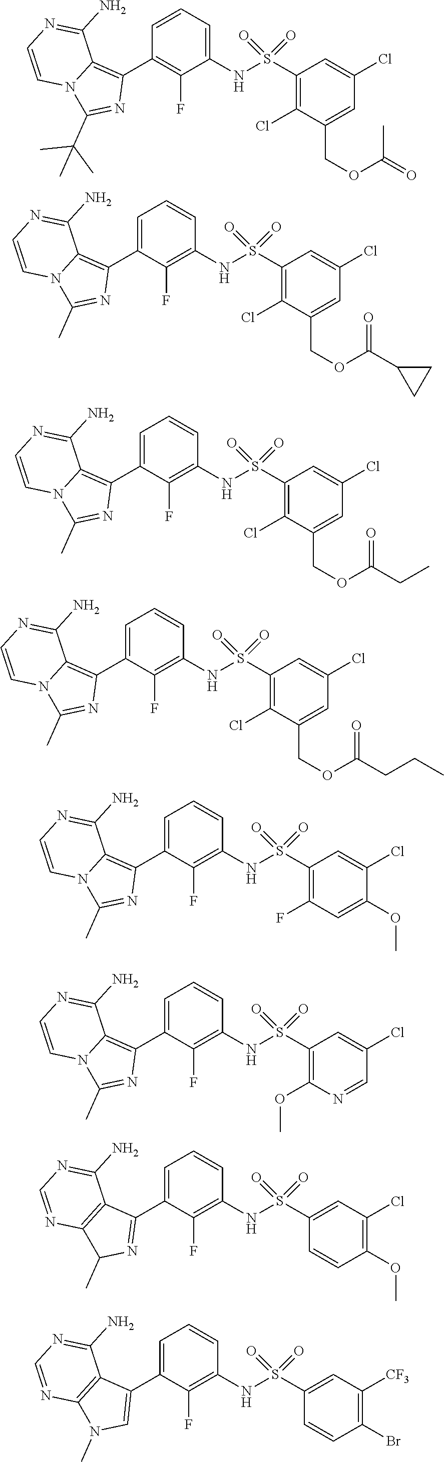 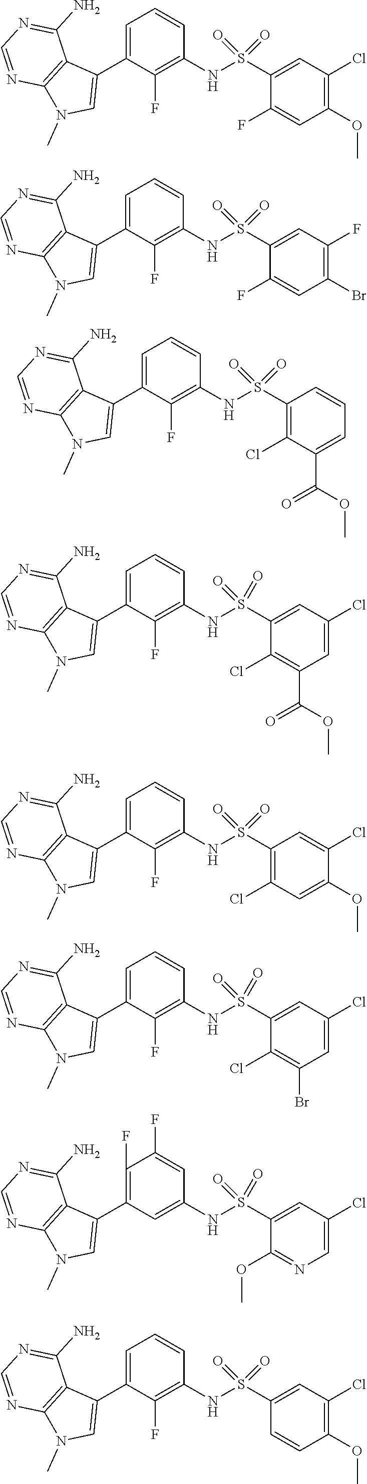 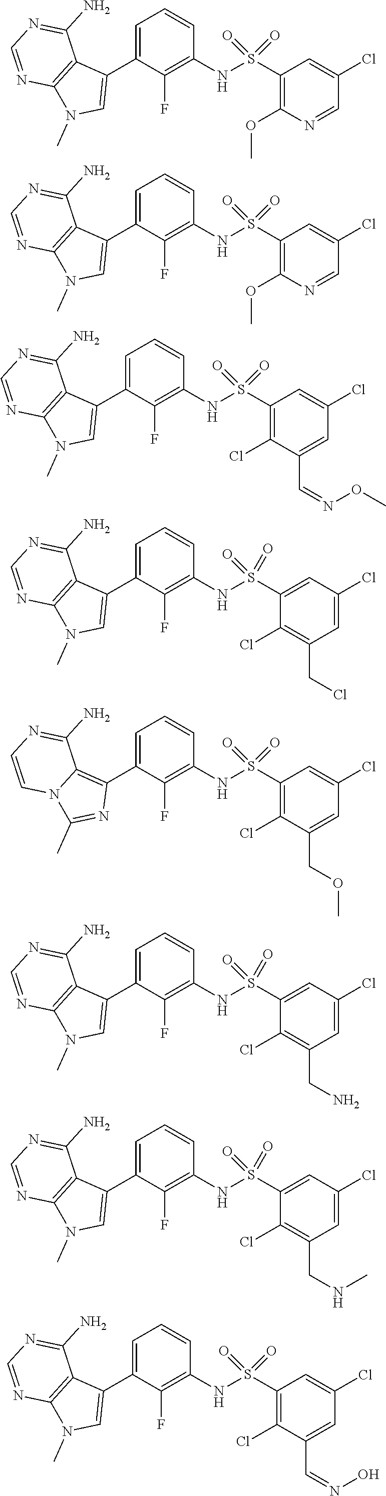
and pharmaceutically acceptable salts, enantiomers, stereoisomers, and tautomers thereof.

34. A pharmaceutical composition comprising a compound according to any one of claims 1-33, or a pharmaceutically acceptable salt thereof, and a pharmaceutically acceptable carrier or excipient.

35. A method of treating a disease caused by a dysregulation of the integrated stress response and/or an unfolded protein response in a patient in need thereof, comprising administering to the patient a therapeutically effective amount of the compound of any one of claims 1-33, or a pharmaceutically acceptable salt thereof, or of the pharmaceutical composition of claim 34.

36. The method of claim 35, wherein the dysregulation of the integrated stress response and/or an unfolded protein response is caused by a kinase selected from the group consisting of PKR-like ER kinase (PERK) kinase and general control nonderepressible 2 (GCN2) kinase.

37. The method of claim 35 or 36, wherein the dysregulation of the integrated stress response and/or the unfolded protein response is caused by GCN2 kinase.

38. The method of claim 35 or 36, wherein the dysregulation of the integrated stress response and/or the unfolded protein response is caused by PERK kinase.

39. A method of modulating the activity of GCN2 kinase in a patient in need thereof, comprising administering to the patient a therapeutically effective amount of the compound of any one of claims 1-33, or a pharmaceutically acceptable salt thereof, or of the pharmaceutical composition of claim 34.

40. A method of modulating the activity of PERK kinase in a patient in need thereof, comprising administering to the patient a therapeutically effective amount of the compound of any one of claims 1-33, or a pharmaceutically acceptable salt thereof, or of the pharmaceutical composition of claim 34.

41. A method of treating a cancer in a patient in need thereof, comprising administering to the patient a therapeutically effective amount of the compound of any one of claims 1-33, or a pharmaceutically acceptable salt thereof, or of the pharmaceutical composition of claim 34.

42. The method of claim 41, wherein the cancer is selected from the group consisting of colorectal cancer, lung cancer, mesothelioma, pancreatic cancer, pharyngeal cancer, laryngeal cancer, esophagus cancer, gastric, duodenal cancer, small intestinal cancer, breast cancer, ovarian cancer, testis tumor, prostate, liver cancer, thyroid cancer, renal cancer, uterine cancer, gestational choriocarcinoma, brain tumor, retinoblastoma, skin cancer, melanoma, sarcoma, fibrosarcoma, malignant bone tumor, urinary bladder cancer, hematologic cancer, leukemia, acute myeloid leukemia, acute lymphoblastic leukemia, chronic lymphocytic leukemia, chronic myeloid leukemia, multiple myeloma, B-cell lymphoma, Follicular lymphoma, mantle cell lymphoma, diffuse large B cell lymphoma, T-cell lymphoma, erythroleukemia, histocyctic lymphoma, Waldenstrom macroglobulinemia, light chain amyloidosis, and malignant lymphoma.

43. A method of treating amyloidosis in a patient in need thereof, comprising administering to the patient a therapeutically effective amount of the compound of any one of claims 1-33, or a pharmaceutically acceptable salt thereof, or of the pharmaceutical composition of claim 34.

44. A method of treating light chain amyloidosis in a patient in need thereof, comprising administering to the patient a therapeutically effective amount of the compound of any one of claims 1-33, or a pharmaceutically acceptable salt thereof, or of the pharmaceutical composition of claim 34.

45. The method of any one of claims 35-44, further comprising administering to the patient a therapeutically effective amount of one or more therapeutic agents.

46. The method of claim 45, wherein the one or more therapeutic agents is selected from the group consisting of an IMiD agent, a proteasome inhibitor, a steroid, an anti-CD38 agent, an anti-CD20 agent, a Bcl-2 inhibitor, a PI3K inhibitor, a Bi-specific antibody, a nucleoside analog, a BTK inhibitor, a DNA alkylating agent, an EZH2 inhibitor, an anthracycline, a topoisomerase inhibitor, a platin, a tyrosine kinase inhibitor, a HDAC inhibitor, a nuclear export inhibitor, an anti-microtubule agent, L-asparaginase, pegylated asparaginase, a PERK inhibitor, a mTOR inhibitor, an immunomodulatory agent, an anti-angiogenic agent, an EGFR inhibitor, a MAPK pathway inhibitor, a MEK inhibitor, an ERK inhibitor, and a Ras inhibitor.

47. The method of claim 45, wherein the one or more therapeutic agents is selected from the group consisting of L-asparaginase, pegaspargase, calaspargase pegol—mnkl, bortezomib, carfilzomib, ixazomib, thalidomide, pomalidomide, lenalidomide, dexamethasone, prednisone, daratumumab, daratumumab/hyaluronidase, isatuximab, rituximab, obinutuzumab, venetoclax, idelalisib, copanlisib, duvelisib, umbralisib, gemcitabine, cytarabine, ibrutinib, acalabrutinib, zanubrutinib, bendamustine, cyclophosphamide, tazemetostat, doxorubicin, daunorubicin, etoposide, oxaloplatin, carboplatin, cisplatin, bosutinib, dasatinib, imatinib, nilotinib, ponatinib, panobinostat, selinexor, vincristine, JZP-458, eryaspase, PF745 (JZP-341), asparaginase Erwinia chrysanthemi (crisantaspase), Escherichia coli asparaginase (colaspase), erlotinib, gefitinib, osimertinib, afatanib, cetuximab, bevacizumab, axitinib, sunitinib, sorafenib, cabozantinib, pazopanib, lenvatinib, vandetanib, regorafenib, nintedanib, apatinib, an anti-PD1 agent, an anti-PDL1 agent, and an anti-CTLA4 agent.

48. A compound of any one of claims 1-33, or a pharmaceutically acceptable salt thereof, or the pharmaceutical composition of claim 34, for use in treating a disease caused by a dysregulation of an integrated stress response and/or the unfolded protein response in a patient in need thereof.

49. The compound or composition for use of claim 48, wherein the dysregulation of the integrated stress response and/or the unfolded protein response is caused by a kinase selected from the group consisting of PERK kinase and GCN2 kinase.

50. The compound or composition for use of claim 48 or 49, wherein the dysregulation of the integrated stress response and/or the unfolded protein response is caused by GCN2 kinase.

51. The compound or composition for use of claim 48 or 49, wherein the dysregulation of the integrated stress response and/or the unfolded protein response is caused by PERK kinase.

52. A compound of any one of claims 1-33, or a pharmaceutically acceptable salt thereof, or the pharmaceutical composition of claim 34, for use in modulating the activity of GCN2 kinase in a patient in need thereof.

53. A compound of any one of claims 1-33, or a pharmaceutically acceptable salt thereof, or the pharmaceutical composition of claim 34, for use in modulating the activity of PERK kinase in a patient in need thereof.

54. A compound of any one of claims 1-33, or a pharmaceutically acceptable salt thereof, or the pharmaceutical composition of claim 34, for use in therapy.

55. A compound of any one of claims 1-33, or a pharmaceutically acceptable salt thereof, or the pharmaceutical composition of claim 34, for use in treating a cancer in a patient in need thereof.

56. The compound or composition for use of claim 55, wherein the cancer is selected from the group consisting of colorectal cancer, lung cancer, mesothelioma, pancreatic cancer, pharyngeal cancer, laryngeal cancer, esophagus cancer, gastric, duodenal cancer, small intestinal cancer, breast cancer, ovarian cancer, testis tumor, prostate, liver cancer, thyroid cancer, renal cancer, uterine cancer, gestational choriocarcinoma, brain tumor, retinoblastoma, skin cancer, melanoma, sarcoma, fibrosarcoma, malignant bone tumor, urinary bladder cancer, hematologic cancer, leukemia, acute myeloid leukemia, acute lymphoblastic leukemia, chronic lymphocytic leukemia, chronic myeloid leukemia, multiple myeloma, B-cell lymphoma, Follicular lymphoma, mantle cell lymphoma, diffuse large B cell lymphoma, T-cell lymphoma, erythroleukemia, histocyctic lymphoma, Waldenstrom macroglobulinemia, and malignant lymphoma.

57. The compound or composition for use of claim 55 or 56, wherein the cancer is leukemia.

58. The compound or composition for use of claim 55 or 56, wherein the cancer is acute myeloid leukemia.

59. The compound or composition for use of claim 55 or 56, wherein the cancer is acute lymphoblastic leukemia.

60. The compound or composition for use of claim 55 or 56, wherein the cancer is fibrosarcoma.

61. The compound or composition for use of claim 55 or 56, wherein the cancer is multiple myeloma.

62. The compound or composition for use of claim 55 or 56, wherein the cancer is lymphoma.

63. The compound or composition for use of claim 55 or 56, wherein the cancer is B-cell lymphoma.

64. The compound or composition for use of claim 55 or 56, wherein the cancer is T cell lymphoma.

65. The compound or composition for use of claim 55 or 56, wherein the cancer is renal cancer.

66. The compound or composition for use of claim 55 or 56, wherein the cancer is lung cancer.

67. The compound or composition for use of claim 55 or 56, wherein the cancer is colorectal cancer.

68. A compound of any one of claims 1-33, or a pharmaceutically acceptable salt thereof, or the pharmaceutical composition of claim 34, for use in treating amyloidosis in a patient in need thereof.

69. A compound of any one of claims 1-33, or a pharmaceutically acceptable salt thereof, or the pharmaceutical composition of claim 34, for use in treating light chain amyloidosis in a patient in need thereof.

70. The compound or composition for use of any one of claims 48-69, further comprising use of one or more therapeutic agents.

71. The compound or composition for use of claim 70, wherein the one or more therapeutic agents is selected from the group consisting of an IMiD agent, a proteasome inhibitor, a steroid, an anti-CD38 agent, an anti-CD20 agent, a Bcl-2 inhibitor, a PI3K inhibitor, a Bi-specific antibody, a nucleoside analog, a BTK inhibitor, a DNA alkylating agent, an EZH2 inhibitor, an anthracycline, a topoisomerase inhibitor, a platin, a tyrosine kinase inhibitor, a HDAC inhibitor, a nuclear export inhibitor, an anti-microtubule agent, L-asparaginase, pegylated asparaginase, a PERK inhibitor, a mTOR inhibitor, an immunomodulatory agent, an anti-angiogenic agent, an EGFR inhibitor, a MAPK pathway inhibitor, a MEK inhibitor, an ERK inhibitor, and a Ras inhibitor.

72. The compound or composition for use of claim 70, wherein the one or more therapeutic agents is selected from the group consisting of L-asparaginase, pegaspargase, calaspargase pegol—mnkl, bortezomib, carfilzomib, ixazomib, thalidomide, pomalidomide, lenalidomide, dexamethasone, prednisone, daratumumab, daratumumab/hyaluronidase, isatuximab, rituximab, obinutuzumab, venetoclax, idelalisib, copanlisib, duvelisib, umbralisib, gemcitabine, cytarabine, ibrutinib, acalabrutinib, zanubrutinib, bendamustine, cyclophosphamide, tazemetostat, doxorubicin, daunorubicin, etoposide, oxaloplatin, carboplatin, cisplatin, bosutinib, dasatinib, imatinib, nilotinib, ponatinib, panobinostat, selinexor, vincristine, JZP-458, eryaspase, PF745 (JZP-341), asparaginase Erwinia chrysanthemi (crisantaspase), Escherichia coli asparaginase (colaspase), erlotinib, gefitinib, osimertinib, afatanib, cetuximab, bevacizumab, axitinib, sunitinib, sorafenib, cabozantinib, pazopanib, lenvatinib, vandetanib, regorafenib, nintedanib, apatinib, an anti-PD1 agent, an anti-PDL1 agent, and an anti-CTLA4 agent.


SEQUENCE LISTING (3 sequences)

SEQ ID NO: 1 - protein, 1894 aa, synthetic construct
MAPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGAMGSGIQRPTSTSSLVMAGGRGAPGRGRDEPPESYPQRQDHELQALEAIYGADFQDLRPDACGPVKEPPEINLVLYPQGLTGEEVYVKVDLRVKCPPTYPDVVPEIELKNAKGLSNESVNLLKSRLEELAKKHCGEVMIFELAYHVQSFLSEHNKPPPKSFHEEMLERRAQEEQQRLLEAKRKEEQEQREILHEIQRRKEEIKEEKKRKEMAKQERLEIASLSNQDHTSKKDPGGHRTAAILHGGSPDFVGNGKHRANSSGRSRRERQYSVCNSEDSPGSCEILYFNMGSPDQLMVHKGKCIGDEQLGKLVYNALETATGGFVLLYEWVLQWQKKMGPFLTSQEKEKIDKCKKQIQGTETEFNSLVKLSHPNVVRYLAMNLKEQDDSIVVDILVEHISGVSLAAHLSHSGPIPVHQLRRYTAQLLSGLDYLHSNSVVHKVLSASNVLVDAEGTVKITDYSISKRLADICKEDVFEQTRVRFSDNALPYKTGKKGDVWRLGLLLLSLSQGQECGEYPVTIPSDLPADFQDFLKKCVCLDDKERWSPQQLLKHSFINPQPKMPLVEQSPEDSGGQDYVETVIPSNRLPSAAFFSETQRQFSRYFIEFEELQLLGKGAFGAVIKVQNKLDGCCYAVKRIPINPASRQFRRIKGEVTLLSRLHHENIVRYYNAWIERHERPAGPGTPPPDSGPLAKDDRAARGQPASDTDGLDSVEAAAPPPILSSSVEWSTSGERSASARFPATGPGSSDDEDDDEDEHGGVFSQSFLPASDSESDIIFDNEDENSKSQNQDEDCNEKNGCHESEPSVTTEAVHYLYIQMEYCEKSTLRDTIDQGLYRDTVRLWRLFREILDGLAYIHEKGMIHRDLKPVNIFLDSDDHVKIGDFGLATDHLAFSADSKQDDQTGDLIKSDPSGHLTGMVGTALYVSPEVQGSTKSAYNQKVDLFSLGIIFFEMSYHPMVTASERIFVLNQLRDPTSPKFPEDFDDGEHAKQKSVISWLLNHDPAKRPTATELLKSELLPPPQMEESELHEVLHHTLTNVDGKAYRTMMAQIFSQRISPAIDYTYDSDILKGNFSIRTAKMQQHVCETIIRIFKRHGAVQLCTPLLLPRNRQIYEHNEAALFMDHSGMLVMLPFDLRIPFARYVARNNILNLKRYCIERVFRPRKLDRFHPKELLECAFDIVTSTTNSFLPTAEIIYTIYEIIQEFPALQERNYSIYLNHTMLLKAILLHCGIPEDKLSQVYIILYDAVTEKLTRREVEAKFCNLSLSSNSLCRLYKFIEQKGDLQDLMPTINSLIKQKTGIAQLVKYGLKDLEEVVGLLKKLGIKLQVLINLGLVYKVQQHNGIIFQFVAFIKRRQRAVPEILAAGGRYDLLIPQFRGPQALGPVPTAIGVSIAIDKISAAVLNMEESVTISSCDLLVVSVGQMSMSRAINLTQKLWTAGITAEIMYDWSQSQEELQEYCRHHEITYVALVSDKEGSHVKVKSFEKERQTEKRVLETELVDHVLQKLRTKVTDERNGREASDNLAVQNLKGSFSNASGLFEIHGATVVPIVSVLAPEKLSASTRRRYETQVQTRLQTSLANLHQKSSEIEILAVDLPKETILQFLSLEWDADEQAFNTTVKQLLSRLPKQRYLKLVCDEIYNIKVEKKVSVLFLYSYRDDYYRILF

SEQ ID NO: 2 - protein, 779 aa, Homo sapiens
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSKYDSVSGEANDSSWNDIKNSGYISRYLTDFEPIQCLGRGGFGVVFEAKNKVDDCNYAIKRIRLPNRELAREKVMREVKALAKLEHPGIVRYFNAWLEAPPEKWQEKMDEIWLKDESTDWPLSSPSPMDAPSVKIRRMDPFSTKEHIEIIAPSPQRSRSFSVGISCDQTSSSESQFSPLEFSGMDHEDISESVDAAYNLQDSCLTDCDVEDGTMDGNDEGHSFELCPSEASPYVRSRERTSSSIVFEDSGCDNASSKEEPKTNRLHIGNHCANKLTAFKPTSSKSSSEATLSISPPRPTTLSLDLTKNTTEKLQPSSPKVYLYIQMQLCRKENLKDWMNGRCTIEERERSVCLHIFLQIAEAVEFLHSKGLMHRDLKPSNIFFTMDDVVKVGDFGLVTAMDQDEEEQTVLTPMPAYARHTGQVGTKLYMSPEQIHGNSYSHKVDIFSLGLILFELLYPFSTQMERVRTLTDVRNLKFPPLFTQKYPCEYVMVQDMLSPSPMERPEAINIIENAVFEDLDFPGKTVLRQRSRSLSSSGTKHSRQSNNSHSPLPSN

SEQ ID NO: 3 - protein, 10 aa, synthetic construct
  MOD_RES 1: note = pyro-Glutamic acid
  MOD_RES 6: note = O-t-butyl-D-Serine
  MOD_RES 10: note = Amidated azaglycine
EHWSYSLRPG